(19) United States
(12) Patent Application Publication
(10) Pub. No.: US 2026/0317822 A1
(43) Pub. Date: Oct. 15, 2026

(54) SINGLE-CELL REGULATORY MULTI-OMICS WITH DEEP MITOCHONDRIAL MUTATION PROFILING (REDEEM)

(71) Applicants: THE CHILDREN'S MEDICAL CENTER CORPORATION, Boston, MA (US); WHITEHEAD INSTITUTE FOR BIOMEDICAL RESEARCH, Cambridge, MA (US)

(72) Inventors: Vijay SANKARAN, Boston, MA (US); Jonathan WEISSMAN, Boston, MA (US); Chen WENG, Boston, MA (US)

(73) Assignees: THE CHILDREN'S MEDICAL CENTER CORPORATION, Boston, MA (US); WHITEHEAD INSTITUTE FOR BIOMEDICAL RESEARCH, Cambridge, MA (US)

(21) Appl. No.: 19/483,725

(22) PCT Filed: May 10, 2024
(86) PCT No.: PCT/US2024/028839

§ 371 Date: Nov. 12, 2025

Related U.S. Application Data
(60) Provisional application No. 63/466,018, filed on May 12, 2023.

(51) Int. Cl. C12Q 1/6876; G01N 1/30

(52) U.S. Cl. CPC C12Q 1/6876; G01N 1/30; C12Q 2600/156; C12Q 2600/158

(57) ABSTRACT

Described herein are methods of detecting and calling somatic mutations in mitochondrial genomes to infer cell lineages in native contexts and to serve as genetic barcodes to measure clonal dynamics in complex cellular populations. Further, somatic mutation in mitochondrial DNA (mtDNA) are tracked by single genomic approaches for simultaneous analysis of single cell lineage and state.

### CROSS-REFERENCE TO RELATED APPLICATIONS

This application is a U.S. National Stage under 35 U.S.C. § 371 of International Application No. PCT/US2024/028839, filed May 10, 2024, which claims benefit under 35 U.S.C. § 119(e) of U.S. Provisional Application No. 63/466,018 filed May 12, 2023, the contents of which are incorporated herein by reference in their entirety.

### GOVERNMENT SUPPORT

This invention was made with government support under Grant Numbers HG009490, CA265726, DK103794, and HL146500, awarded by the National Institutes of Health. The Government has certain rights in the invention.

### SEQUENCE LISTING

The instant application contains a Sequence Listing which has been submitted electronically in XML format and is hereby incorporated by reference in its entirety. Said XML copy, created on Jun. 13, 2024, is named “701039-000113WOPT_SL.xml” and is 38,694 bytes in size.

### TECHNICAL FIELD

The technology described herein relates to methods of determining or tracking cell lineages at high resolution.

### BACKGROUND

All cells in the human body are derived from the zygote, but there is lack of a detailed map integrating cell division (lineage) and differentiation (fate) and their dynamics from stem cells to their differentiated progeny. Such a map would significantly expand the understanding of cellular processes underlying human development, tissue homeostasis, and disease. In model organisms, most approaches to date rely on an engineered genetic label to tag individual cells or populations with heritable marks such as fluorescent or reporter genes, high diversity DNA barcode libraries, mobile transposable elements, Polylox/Cre, or CRISPR-based barcodes. However, these genetic manipulations are not readily possible in humans. Instead, in human tissues in vivo, naturally occurring somatic mutations, including single nucleotide variants (SNVs), copy number variants (CNVs), and variation in short tandem repeat sequences (microsatellites or STRs), which are stably propagated to daughter cells, but absent in distantly related cells are relied upon. These approaches are costly, difficult to apply at scale, have substantial error rates, and do not provide information on cell state.

Technologies that can simultaneously provide rich cell state readouts in single cells, while also yielding detailed genealogical information from natural cellular barcodes would, in principle, overcome this limitation. Mitochondrial DNA (mtDNA) mutations have been shown to serve as natural cellular barcodes in humans. However, existing methods can only detect a limited subset of mtDNA mutations, hampering the ability to resolve fine-scale subclonal relationships and hierarchies.

### SUMMARY

Described herein are methods for high resolution single-cell lineage tracing with rich multiomics readouts. These methods permit refined studies of clonal dynamics across a range of tissues in human health and disease.

It is recognized herein that methods of fixing and permeabilizing whole cells affect the efficiency with which mtDNA differences can be detected, e.g., in a tissue or tissue section. Accordingly, described herein, in one aspect, are methods of determining a cell lineage or a cell state of a single cell in a multicellular eukaryotic organism with using fixation and permeabilization of whole cells that improves the ability to detect and sequence nucleic acids, including mtDNA, in situ.

In one aspect, described herein are methods of determining a cell lineage or a cell state of a single cell in a multicellular eukaryotic organism, comprising:
detecting mutations in mitochondrial genome consensus sequencing reads of single cells obtained from a subject to generate mitochondrial genome mutation data, the mitochondrial genome consensus sequencing reads having been obtained by single-cell targeted sequencing of the mitochondrial genome and unique molecular identifier (UMI) sequencing error correction; and
assigning a lineage and/or status group to the single cells based on at least the mutations in the mitochondrial genome mutation data.

In one embodiment of this and all other aspects described herein, the method of determining a cell lineage or a cell state of a single cell in a multicellular eukaryotic organism further comprises:
measuring gene expression levels for the single cells to generate gene expression level data; and
detecting chromatin accessibility for the single cells to generate chromatin accessibility data;
wherein the mitochondrial genome mutation data, the gene expression level data, and the chromatin accessibility data of a particular single cell are associated with each other.

In another aspect, described herein are methods of determining a cell lineage or a cell state of a single cell in a multicellular eukaryotic organism, comprising:
detecting mutations in mitochondrial genome consensus sequencing reads of single cells obtained from a subject to generate mitochondrial genome mutation data;
measuring gene expression levels for the single cells to generate gene expression level data; and
detecting chromatin accessibility for the single cells to generate chromatin accessibility data;
wherein the mitochondrial genome mutation data, the gene expression level data, and the chromatin accessibility data of a particular single cell are associated with each other.

In one embodiment of this and all other aspects described herein, the method of determining a cell lineage or a cell state of a single cell in a multicellular eukaryotic organism further comprises assigning a lineage and/or status group to the single cells based on the mutations in the mitochondrial genome mutation data, the gene expression level data, and the chromatin accessibility data.

In another embodiment of this and all other aspects described herein, the method of determining a cell lineage or a cell state of a single cell in a multicellular eukaryotic organism further comprises generating a phylogenetic tree for the single cells based on the mutations in the mitochondrial genome mutation data, the gene expression level data, and the chromatin accessibility data of the single cells. In another embodiment of this and all other aspects described herein, the method further comprises displaying the phylogenetic tree.

In another embodiment of this and all other aspects described herein, the detecting chromatin accessibility in the single cells comprises single cell ATAC-seq (scATAC-seq) analysis.

In another embodiment of this and all other aspects described herein, the single-cell targeted sequencing of the mitochondrial genome comprises hybrid capture of targeted sequences using one or more sets of probes. In another embodiment of this and all other aspects described herein, the probes are DNA probes. In another embodiment, the probes are biotinylated probes.

In another embodiment of this and all other aspects described herein, the mitochondrial genome consensus sequencing reads are obtained from a scATAC-seq library.

In another embodiment of this and all other aspects described herein, the UMI is an endogenous UMI (eUMI).

In another embodiment of this and all other aspects described herein, method comprises sequencing the mitochondrial genome of the single cells to generate mitochondrial genome sequencing reads, and aligning the mitochondrial genome sequencing reads to obtain mitochondrial genome consensus sequencing reads.

In another embodiment of this and all other aspects described herein, the method comprises fixing the single cells prior to the sequencing. In another embodiment, the single cells are fixed in a fixation buffer comprising an aldehyde. In another embodiment, the fixation buffer comprises albumin.

In another embodiment of this and all other aspects described herein, the method comprises permeabilizing the single cells prior to the sequencing. In another embodiment, the single cells are permeabilized in a permeabilization buffer comprising a surfactant.

In another aspect, described herein is a method of fixing and permeabilizing whole cell, comprising:
(a) fixing the cells by contacting the cells with a fixation buffer comprising an aldehyde and albumin; and
(b) permeabilizing the cells, after the fixing, by contacting the cells with a permeabilization buffer comprising a surfactant.

In another embodiment of this and all other aspects described herein, the albumin is bovine serum albumin (BSA). In another embodiment, the fixation buffer comprises 0.001%-2% bovine serum albumin (BSA). In another embodiment, the fixation buffer comprises 0.01% to 0.04% BSA. In another embodiment, the aldehyde is formaldehyde. In another embodiment, the fixation buffer comprises 0.01% to 1% formaldehyde. In another embodiment, the fixation buffer comprises RNase inhibitor (RI). In another embodiment, the fixation buffer comprises 0.1 to 1 U/μL RI.

In another embodiment of this and all other aspects described herein, the surfactant is NP-40. In another embodiment, the permeabilization buffer comprises MgCl₂. In another embodiment, the permeabilization buffer comprises DTT. In another embodiment, the permeabilization buffer comprises BSA. In another embodiment, the permeabilization buffer comprises 0.0001% to 2% BSA.

In another embodiment of this and all other aspects described herein, the method further comprises isolating nucleic acid molecules from the whole cells. In another embodiment, the method comprises generating a sequencing library from the nucleic acid molecules. In another embodiment, the sequencing library is a scATAC-seq sequencing library, a scRNA-seq sequencing library, or a sequencing library obtained from a mitochondrial genome. In another embodiment, the method further comprising sequencing the sequencing library.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, A D A M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

“Lineages” as used herein refers to a term to describe cells with a common ancestry, for example cells that are derived from the same parental or stem cell.

As used herein “calling of a mutation” or “mutation calling” refers to identifying nucleobase differences between two nucleic acid samples using sequence data.

The term “tissue” refers to a group or layer of similarly specialized cells which together perform certain special functions. The term “tissue-specific” refers to a source or defining characteristic of cells from a specific tissue or the genes they express.

The term “wild type” refers to the naturally-occurring polynucleotide sequence encoding a protein, or a portion thereof, or protein sequence, or portion thereof, respectively, as it normally exists in vivo.

The methods described herein permit the sensitive detection of changes in mtDNA sequences in a given cell or cell population relative to other cells in a subject or in a tissue or cell population of a subject that permit the tracing of the lineage of that given cell or cell population. In this context, a “mutation” refers to any change or variation (deletion, substitution, addition, or sequence alteration of one nucleotide or more) in the mtDNA of a given cell or cell population relative to that in another cell or cell population in that subject or tissue or cell population in that subject. A mutation can, but need not change protein coding sequence. The term “variant” is used interchangeably with “mutant” herein in regard to mtDNA sequences.

The terms “increased”, “increase”, “enhance”, “activate” are all used herein to refer to an increase by a statistically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to a reference level, for example, a decrease of at least about 20%, or at least about 30%, or at least about 40%, or at least about 50%, or at least about 60%, or at least about 70%, or at least about 80%, or at least about 90%, or at least about 95% as compared to a reference level. As used herein, “reduction” or “inhibition” does not encompass a complete inhibition or reduction as compared to a reference level. “Complete inhibition” is a 100% inhibition as compared to a reference level.

As used herein, a “reference level” refers to the level or value for a given parameter against which one compares the level or value in a given sample or situation to determine whether the level or value has changed in a meaningful way. A reference level can be a level in or from a sample that is not treated to change the parameter. A reference level can alternatively be a level in or from a normal or otherwise unaffected sample. A reference level can alternatively be a level in or from a sample obtained from a subject at a prior time point, for example, prior to a given treatment. A reference level can also refer to a level in one cell relative to another cell of a given population of cells.

As used herein, an “appropriate control” refers to an untreated, otherwise identical sample, cell or population that serves as a reference relative to a treated sample, cell or population.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

The terms “subject” and “individual” are used interchangeably herein, and refer to an animal, for example a human, being evaluated with regard to cell lineages, whether for research, diagnosis or treatment. The terms “non-human animals” and “non-human mammals” are used interchangeably herein, and include, for example, mammals such as rats, mice, rabbits, sheep, cats, dogs, cows, pigs, and non-human primates. Mammals other than humans can be advantageously used as subjects that represent animal models for diagnosis or treatment of disease. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

As used herein, a “subject in need” of diagnosis or treatment for a particular condition can be a subject having that condition, diagnosed as having that condition, or at risk of developing that condition.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

### BRIEF DESCRIPTION OF THE DRAWINGS

FIGS. 1A-1F demonstrates single-cell deep mtDNA mutation detection with joint multi-omics. (FIG. 1A) Schematic of ReDeeM workflow. (FIG. 1B) Comparison of mtDNA copy number and UMI group size per cell before and after mtDNA enrichment. UMI group size is the number of raw reads in each UMI group. (FIG. 1C) Comparison of confident mtDNA mutation number between before mtDNA enrichment (via mgatk) and after enrichment (via UMI consensus calling). (FIG. 1D) Mutational signatures in each class of mononucleotide and trinucleotide change by the heavy (H) and light (L) strands, under optimised protocol. Mutational signatures are compared across unfiltered (top), 4831 mtDNA mutation via UMI consensus calling (mid) and a previously reported mtDNA mutation signature in bulk. (FIG. 1E) Distribution of the number of confident mtDNA mutations per cell before (via mgatk) and after mtDNA enrichment (via UMI consensus calling). (FIG. 1F) Network connectedness analysis before (via mgatk) and after mtDNA enrichment (via UMI consensus calling). Each dot represents a cell and each line connects cells with shared mutations. The connectedness is defined as the number of “neighbour” (or “neighbor”) cells that share at least 1 mtDNA mutation with any given cell.

FIGS. 2A-2G demonstrate fine-scale lineage tracing with simultaneous state profiling for human hematopoiesis at steady state. (FIG. 2A) Schematic of the experimental design. (FIG. 2B) Phylogenetic tree for hematopoietic cells of young-1 generated based on the number of shared mtDNA mutations using neighbour joining algorithm. The numbers of sharable mtDNA mutations for each cell are indicated. (FIG. 2C) Joint multi-omics clustering of young-1 (for the same cells from panel b). Weighted nearest neighbour UMAP (wnnUMAP) shows combined ATAC and RNA profile for 11,019 single cells. (FIG. 2D) Analysis of (Left) chromatin accessibility, (middle) mRNA expression, and (right) DNA binding activity of SPI1 or GATA1 transcription factors in HSC differentiating toward myeloid versus mega-erythroid trajectories. (FIG. 2E) Measurement of mtDNA mutation burdens across different cell types. (FIG. 2F) Analysis of cell type origins based on shared mtDNA mutations. Color intensity indicates the proportion of each target cell type (horizontal axis) within the mtDNA mutation-based k nearest neighbourhood (KNN) of the query cell type (vertical axis). (FIG. 2G) Integrative analysis between phylogenetic tree and multi-omics-based cell types. Examples of cell type restricted local clades are highlighted (clade i to clade viii).

FIGS. 3A-3G demonstrates HSC clonal architecture and clonal-dependent cell state biases. (FIG. 3A) Schematic of the experimental design. Bone marrow samples are obtained from the same individual at two different time points, 4 months apart. HSCs are enriched by FACS and further defined by single-cell gene expression markers. (FIG. 3B) Validation of HSCs classification. Gene expression of other independent HSC markers are shown. (FIG. 3C) Subpopulations of HSCs based on single cell RNA- and ATAC-space as well as combined WNN space. (FIG. 3D) Phylogenetic tree of HSCs (Young-1) sampled from each of the two time points, based on shared mtDNA mutations. (FIG. 3E) Examples of differentially expressed genes across HSC subpopulations. (FIG. 3F) Overlap analysis between the HSC clonal groups and the HSC state subpopulations. Color intensity indicates the enrichment p values. (FIG. 3G) Comparison of enrichment (as in panel f) across the two time points. Enrichment fold change is compared, Color intensity indicates the combined enrichment p-value.

FIGS. 4A-4F demonstrates HSC clonal output activity and lineage biases. (FIG. 4A) Schematic of the strategy that assigns the progeny cells to HSC clonal groups using network propagation via mtDNA mutation-based cellular networks (SCAVENGE-L). (FIG. 4B) Summary of HSC clonal output activity (the number of progeny cells from each HSC clone) across two sampling time points. The progeny numbers are normalized to the HSC clone size. (FIG. 4C) Correlation analysis of the clonal output activities between the two time points. (FIG. 4D) The output contribution from each HSC clone, at both time points (T1=timepoint 1; T2=timepoint 2) in donor young-1 and donor young-2, ranked from highest contributing to lowest contributing to the total progeny population. The dashed line indicates the expectation of equal contribution from all clones, (FIG. 4E) For each HSC clonal group, the percentages of the progeny that differentiate into one of the four main lineages are shown: megakaryocyte (MK), lymphoid, Mega-Erythroid (ME), and myeloid (Mye) cells. The significance of clonal lineage biases is indicated (*0.01-0.05, **0.001-0.01, ***<0.001). On the top, the fold changes of clonal lineage biases for each clone are indicated for both timepoints of sampling. (FIG. 4F) Correlation between HSC clonal output activity and clonal lineage biases.

FIGS. 5A-5G demonstrates clonal structure alteration of human hematopoiesis with ageing. (FIG. 5A) mtDNA mutation burden between young and aged donors across different cell types. (FIGS. 5B-5C) Phylogenetic trees of the HSC clones from (FIG. 5B) young, and (FIG. 5C) aged donors. Clonal groups are indicated by different colours on outer rings (FIG. 5D) Contribution of each clone to the total population, in two young and two aged donors. (FIG. 5E) Shannon diversity index of the clonal composition, between young versus old donors. (FIG. 5F) Mapping the single-cell fitness score and the cells with loss of Y chromosome (LOY) on the phylogenetic tree for aged donor 1. Raw and smoothed LOY cell distribution are shown in outer rings. (FIG. 5G) Cell type contributions in each expanded clade. The grey area indicates the expected balanced cell type distribution.

FIGS. 6A-6G shows the ReDeeM protocol optimization and quality control. (FIG. 6A) The recovery efficiency rate of mtDNA, ATAC-seq, and RNA-seq across 10 different experimental conditions in fixation, permeabilization, and tagmentation was determined to identify the optimal condition. A (0.1% FA, 10 min RT, 0.1% NP40 5 min, 1% BSA) B (0.1% FA, 10 min RT, 0.1% NP40 5 min, 0.01% BSA); C (1% PFA, 30 min RT, 0.1% NP40 5 min, 1% BSA); D (1% PFA, 30 min RT, 0.1% NP40 5 min, 0.01% BSA); E (1% PFA, 30 min RT, No lysis, 0.01% BSA). All conditions are tested with and without glyco-diosgenin (GDN) in tagmentation. (FIG. 6B) Human-mouse species mix experiment. 4% of the droplets were doublets in all three modalities. (FIGS. 6C-6D) Tn5 insertion size distribution (FIG. 6C) in nuclear genome DNA or (FIG. 6D) in mitochondrial genomes. (FIG. 6E) Aggregated ATAC-seq fragments near transcription start sites (TSS). (FIG. 6F) Distribution of joint-profiled single cells (dots) based on the number of ATAC nuclear fragments versus the number of RNA transcripts UMI per cell. (FIG. 6G) Schematic of the data structure

FIGS. 7A-7L show improved mtDNA coverage and mutation detection with ReDeeM pipeline. (FIG. 7A) Percentage of total reads mapped to mitochondrial genome per cell, before and after mtDNA enrichment. (FIG. 7B) Averaged mitochondrial genome coverage per cell at each position before and after mtDNA enrichment (FIG. 7C) Variant calling before enrichment using mgatk. 311 confident variants are identified. (FIG. 7D) Justification of cell barcode plus starting and ending sequences as endogenous UMI (eUMI) by simulation. The number of mtDNA copies per cell was simulated (x-axis) and the confliction rate was calculated accordingly. (FIG. 7E) Statistical modelling for strand bias removal using a binomial distribution model. White zone includes mutations for which the null hypothesis (no strand biases) hold true, whereas red zone indicates rejection of this null hypothesis (both p<0.01 and fold change >+/−2) in favour of strand biases. (FIG. 7F) More than 90% of bases were overlapped by both reads in a paired-end sequencing. (FIG. 7G) For a given mutation, the number of cells carrying it (x-axis) versus the maximum number of mutant alleles per cell (y-axis) are shown. Heteroplasmic mutations in black dots and homoplasmic mutations in grey dots. (FIGS. 7H-7K) Examples of quality control for mtDNA mutations. (FIG. 7H) The number of supporting reads for each mtDNA molecule (UMI group) containing the mutation. (FIG. 7I) Consensus score: the percentage of mutant reads in a UMI group. Mutations with consensus scores of less than 75% were discarded. (FIG. 7J) For a given mutation, the proportion of molecules that were covered by both reads in a paired-end sequencing. (FIG. 7K) Strand bias ratio (a ratio of 1 indicating no strand bias). (FIG. 7L) Workflow of the ReDeeM variant calling pipeline.

FIGS. 8A-8E demonstrate validation of ReDeeM lineage tracing via dual-tracer experiment. (FIG. 8A) The design of the dual lineage-tracer experiment with a Kras;Trp53(KP)-drive lung adenocarcinoma lineage-tracer mouse model. 6 Independent tumours were profiled. (FIG. 8B) Crispr indel-based lineage relationships were computed with weighted hamming distance and visualised by multidimensional scaling (MDS). Only tumour 1 is shown. (FIG. 8C) Example mtDNA somatic mutations that agree with the Crispr indel-based MDS map are highlighted. (FIGS. 8D-8E), Validation of ReDeeM-based neighbourhood. For any given cell, the neighbourhood (k=20) is defined by shared mtDNA mutations, which is examined by “agreement of closeness” that is defined as the rank % closer than random in Crispr indel-based neighbourhood. (FIG. 8D) The distribution of “agreement of closeness” is grouped by cells with different mtDNA mutation detectability. The more mtDNA mutations detected per cell, the better agreement with Crispr indel-based method. (FIG. 8E) The phylogenetic trees based on mtDNA mutations are illustrated. The “agreement of closeness” (middle) as well as mutation number (bottom) are shown for every single cell (each leaf). The p values are computed by permutation 1000 times.

FIGS. 9A-9B demonstrate informative mtDNA mutations that support phylogenetic and lineage analysis. (FIG. 9A) Top 1000 informative mtDNA mutations supporting the phylogenetic tree. Zoom-in views are examples which demonstrate that many of the fine-scale phylogenetic structures are supported by more than one mtDNA mutation. (FIG. 9B) Examples of mtDNA mutations with restricted lineage preference are highlighted on cell-state UMAP,

FIGS. 10A-10E demonstrate validation of fine-scale human hematopoiesis lineage tracing on donor young-2. Related to FIG. 2. Same analyses of FIG. 2b,c,e-g were reproduced on donor young-2. (FIG. 10A) Phylogenetic tree for hematopoietic cells of young-2. The number of sharable mtDNA mutations for each cell is indicated. (FIG. 10B) Joint multi-omics clustering of young-2. Weighted-nearest neighbour UMAP (wnnUMAP) shows combined ATAC and RNA profile for each cell. (FIG. 10C) Mitochondrial mutation per cell in cells at different stages of differentiation. (FIG. 10D) Analysis of cell type origins based on shared mtDNA mutations. (FIG. 10E) Integrative analysis between phylogenetic tree and multi-omics-based cell types. Examples of cell type restricted local clades are shown (clade i to clade viii).

FIGS. 11A-11I demonstrate single-cell multiomics analyses of HSC subpopulations in donor young-1. (FIGS. 11A-11E) Identifying hematopoietic stem cells (HSC) based on molecular markers for young-1 (FIG. 11A) Unsupervised clustering of hematopoietic stem and progenitor cells (CD34+ cells, and, CD34⁺CD45RA⁻CD90⁺ cells based on joint ATAC and RNA modalities. (FIG. 11B) Expression of HLF mRNA level, a molecular marker of HSCs, in each cluster. (FIG. 11C) Distribution of HLF expressing levels on wnnUMAP (FIG. 11D) Cut-offs to define HSCs from CD34⁺CD45RA⁻CD90⁺ cells using HLF, CRHBP, and CD34 expression levels. (FIG. 11E) Highlighting defined HSCs on wnnUMAP. (FIG. 11F) multi-omics-based distribution of HSCs from samples obtained on two different time points. (FIG. 11G) Examples of differentially expressed genes(blue) and differential transcription factor binding motifs (brown) across HSC subpopulations on the wnnUMAP. (FIG. 11H) Top examples of HSC subpopulation-specific gene expression profiles, based on RNA modality (FIG. 11I) Top examples of HSC subpopulation-specific transcription factor DNA binding motifs, based on ATAC modality. Triangles indicate the ones discussed in the manuscript.

FIGS. 12A-12J demonstrate single-cell multiomics and phylogenetic analyses of HSC subpopulations in donor young-2. Related to FIG. 3 and FIG. 11 (FIGS. 12A-12E) Identifying hematopoietic stem cells (HSC) based on molecular markers for donor young-2. (FIG. 12F) Reproduce analysis of FIG. 3c in young-2. Subpopulations of HSCs based on single cell RNA- and ATAC-space as well as weighted nearest neighbour space. (FIG. 12G) Reproduce analysis of Extended Data FIG. 6h in young-2. Top examples of HSC subpopulation-specific gene expression profiles, based on RNA modality. (FIG. 12H) Reproduce analysis of FIG. 11i in young-2. Top examples of HSC subpopulation-specific transcription factor DNA binding motifs, based on ATAC modality. (FIGS. 12I-12J) Reproduce analysis of FIG. 3d,f in young-2. (FIG. 12I) Phylogenetic tree of HSCs based on shared mtDNA mutations. (FIG. 12J) Overlap analysis between the HSC clonal groups and the HSC state subpopulations. Colour intensity indicates the enrichment p values.

FIGS. 13A-13H demonstrate validation of network propagation-based clonal output analysis. (FIG. 13A) The assignment probability of progeny cell examples (20 random cells) to all 78 HSC clones. The probability is determined from network propagation. The HSC clones with highest probability are colored in red, but only the ones above the threshold 0.7 are assigned in downstream analysis. Figure discloses SEO ID NOS 9-28, respectively, in order of appearance. (FIG. 13B) Benchmarking the assignment accuracy in donor young-1. The predicted clonal assignments for HSCs using network propagation are compared to the “ground truth”, the actual HSC clones. The accuracy is computed for different probability thresholds. (FIG. 13C) The histogram that summarises the number of cells with different maximum assignment probability in donor young-1. (FIGS. 13D-13E) Reproduce the same analysis of b and c for donor young-2. (FIGS. 13F-13H) Reproduce the analysis of FIG. 4b,e,f in donor young-2. (FIG. 13F) Summary of HSC clonal output activity in young-2. (FIG. 13G) For each HSC clonal group, the percentage of the progenies that differentiated into one of four main lineages: megakaryocyte (MK), lymphoid, Mega-Erythroid (ME), and myeloid (Mye) cells. (FIG. 13H) Correlation between HSC clonal output activity and clonal lineage biases in young-2.

FIGS. 14A-14J demonstrate quantification and validation of clonal structure alteration in hematopoiesis. (FIG. 14A) Identification of “expanded clades” in young and old donors, which are defined as the clades with more than 0.5% of total cell numbers and with expansion significance lower than 0.01. (FIG. 14B) The proportion of cells that are contributed from expanded clades are summarised for each donor. (FIG. 14C) Related to FIG. 5d, Measuring the clonal contribution by changing the parameters that affects the definition of “clones”. The parameters involved are m (minimum number of cells as a clone, default is 50), n(minimum number of cumulative variants on the branch to cut, default is 1), p (The probability of the variant to be assigned, default is 0.6), and D (Dump small clones with less than D cells). (FIG. 14D) Related to FIG. 5f. Single-cell fitness analysis in donor aged-2. (FIG. 14E) Related to FIG. 5g. Cell type contributions for each expanded clade for donor aged-2. (FIGS. 14F-14H) Cell-cycle gene expression analysis for expanded versus non-expanded cells. (FIGS. 14I-14J), Identification of cells with loss of chromosome Y. (FIG. 14I) shows the normalised reads number distribution on chromosome Y per cell across different donors. (FIG. 14J) binomial test to identify cells that significantly lose chromosome Y. The cutoff of fold-change is 0.1 and q-value is 0.001.

### DETAILED DESCRIPTION

Embodiments disclosed herein provide methods of detecting somatic mutations in mitochondrial genomes to retrospectively infer cell lineages in native contexts and to serve as genetic barcodes to measure clonal dynamics in complex cellular populations, as well using single cell measurements to determine gene expression and chromatin accessibility to analyze cell state.

Lineage tracing provides unprecedented insights into the fate of individual cells and their progeny in complex organisms. While effective genetic approaches have been developed in vitro and in animal models, these cannot be used to interrogate human physiology in vivo. Instead, naturally occurring somatic mutations have been utilized to infer clonality and lineal relationships between cells in human tissues, but current approaches are limited by high error rates and scale, and provide little information about the state or function of the cells. Described herein are methods to detect and call somatic mutations in mitochondrial DNA (mtDNA) that can be tracked by current single cell genomic approaches for simultaneous analysis of single cell lineage and state.

As disclosed herein, somatic mtDNA mutations are leveraged as natural genetic barcodes and are used as clonal markers to infer lineal relationships. The lineage of human cells is traced by somatic mtDNA mutations in a native context both in vitro and in vivo. Further mitochondrial mutations are readily detected with contemporary single cell transcriptomic and epigenomic technologies to concomitantly capture gene expression profiles and chromatin accessibility, respectively. The approach can allow lineage tracing at a 100- to 1,000-fold greater scale than with single cell whole genome sequencing, while providing information on cell state, permitting one to chart detailed cell lineage and fate maps in human health and disease.

As disclosed herein, single-cell RNA-Seq (scRNA-seq) and single cell ATAC-Seq (scATAC-Seq) are used to capture mitochondrial RNA and DNA sequences and to provide a reliable measurement of mtDNA allelic heteroplasmy in individual cells along with their transcriptional or epigenomic profile. Mitochondrial mutations in human cells are stably propagated both in vitro and in vivo in physiological settings. These heteroplasmic mutations are used to retrospectively infer cell lineages in native contexts and serve as genetic barcodes to measure clonal dynamics in complex cellular populations. Finally, it is demonstrated that mutations are ubiquitous across human tissues.

### Mitochondrial Genomes

Mitochondria are dynamic organelles that are present in almost all eukaryotic cells and play a crucial role in several cellular pathways (see, e.g., Taanman, Biochimica et Biophysica Acta (BBA)—Bioenergetics, Volume 1410, Issue 2, 9 Feb. 1999, Pages 103-123). The human mitochondrial DNA (mtDNA) is a double-stranded, circular molecule of 16,569 bp and contains 37 genes coding for two rRNAs, 22 tRNAs and 13 polypeptides. These mRNAs are transcribed and then translated within the mitochondrial matrix by a dedicated, unique, and highly specialized machinery.

The term “mitochondrial DNA,” which is used interchangeably with “mtDNA,” refers the DNA of the mitochondrion, a structure situated in the cytoplasm of the cell rather than in the nucleus (where all the other chromosomes are located). In vivo, all mtDNA is inherited from the mother. There are 2 to 10 copies of the mtDNA genome in each mitochondrion. mtDNA is a double-stranded, circular molecule. It is very small relative to the chromosomes in the nucleus and includes only a limited number of genes, such as those encoding a number of the subunits in the mitochondrial respiratory-chain complex and the genes for some ribosomal RNAs and transfer RNAs. A cell includes mtDNA derived from the continued replication of cytoplasmically based mitochondria, which in the case of spindle transfer are based in the recipient cytoplast.

Methods to Optimize mtDNA Yield

In certain embodiments, described herein are methods for optimizing mtDNA yield from cells for sequencing. In one aspect, described herein is a method of fixing and permeabilizing whole cells, comprising (a) fixing the cells by contacting them with a buffer comprising formaldehyde, bovine serum albumin, and RNase inhibitor and (b) permeabilizing the fixed cells by contacting them with a buffer comprising Tris, NaCl, MgCl₂, NP40, DTT, BSA and RNase Inhibitor.

In some embodiments of any of the aspects, the buffer that contacts the cells during fixation comprises 0.01%-1% formaldehyde (FA), 0.02%-0.9/o FA, 0.03/6-0.8% FA, 0.04%-0.7% FA, 0.05/6-0.6% FA, 0.06%-0.5% FA, 0.07%-0.4% FA, 0.08%-0.3% FA, 0.09%-0.2% FA, or 0.1% FA.

In some embodiments of any of the aspects, the buffer that contacts the cells during fixation comprises 0.01%-2% bovine serum albumin (BSA), 0.02%-1.9/o BSA, 0.03%-1.8% BSA, 0.04%-1.7% BSA, 0.05/6-1.6% BSA, 0.06%-1.5% BSA, 0.07/6-1.4% BSA, 0.08%-1.3% BSA, 0.09/6-1.2% BSA, 0.1%-1.1% BSA, 0.2%-1% BSA, 0.3%-0.9/o BSA, 0.4/6-0.8%, 0.5%-0.7% BSA, or 0.6% BSA.

In some embodiments, the concentration of BSA in the fixation buffer is at least 0.005%, but less than 1.0%, less than 0.9%, less than 0.8%, less than 0.7%, less than 0.6%, less than 0.5%, less than 0.4%, less than 0.3%, less than 0.2%, 0.1% or less, 0.09% or less, 0.08% or less, 0.07% or less, 0.06% or less, 0.05% or less, 0.04% or less, 0.03% or less, 0.02% or less, 0.01% or less, 0.009% or less, 0.008% or less, 0.007% or less, or 0.006% or less.

In some embodiments of any of the aspects, the buffer that contacts the cells during fixation comprises 0.1-1 U/μl RNase inhibitor (RI), 0.2-0.9 U/μl RI, 0.3-0.8 U/μl RI, 0.4-0.7 U/μl RI, 0.5-0.6 U/μl RI, or 0.45 U/μl RI.

In certain embodiments, the buffer that contacts the cells during fixation comprises 0.1% FA, 0.04% BSA, and 0.2 U/μL RI.

In certain embodiments, the buffer that contacts the cells during fixation comprises 0.1% FA, 0.01% BSA, and 0.2 U/μL RI.

In some embodiments of any of the aspects, the fixation of whole cells takes place at room temperature. As used herein, the term “room temperature” refers to the ambient temperature of a space, which is typically 20° C.-22° C.

In some embodiments of any of the aspects, the fixation of whole cells takes place at room temperature for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes at least 19 minutes, or at least 20 minutes.

In some embodiments of any of the aspects, the buffer that contacts the cells during permeabilization comprises 10 mM Tris. In some embodiments of any of the aspects, the buffer permeabilization buffer comprises 5 mM Tris, 6 mM Tris, 7 mM Tris, 8 mM Tris, 9 mM Tris, 10 mM Tris, 11 mM Tris, 12 mM Tris, 13 mM Tris, 14 mM Tris, or 15 mM Tris.

In some embodiments of any of the aspects, the buffer that contacts the cells during permeabilization comprises 10 mM NaCl. In some embodiments of any of the aspects, the buffer permeabilization buffer comprises 5 mM NaCl, 6 mM NaCl, 7 mM NaCl, 8 mM NaCl, 9 mM NaCl, 10 mM NaCl, 11 mM NaCl, 12 mM NaCl, 13 mM NaCl, 14 mM NaCl, or 15 mM NaCl.

In some embodiments of any of the aspects, the buffer that contacts the cells during permeabilization comprises 2 mM MgCl₂. In some embodiments of any of the aspects, the buffer that contacts the cells during permeabilization comprises 3 mM MgCl₂. In some embodiments of any of the aspects, the buffer permeabilization buffer comprises 1 mM MgCl₂, 2 mM MgCl₂, 3 mM MgCl₂, 4 mM Na MgCl₂Cl, or 5 mM MgCl₂.

In some embodiments of any of the aspects, the buffer that contacts the cells during permeabilization comprises 0.1% NP40. In some embodiments of any of the aspects, the buffer permeabilization buffer comprises 0.01%-1% NP40, 0.02/6-0.9% NP40, 0.03%-0.8% NP40, 0.04%-0.7% NP40, 0.05%-0.6% NP40, 0.06%-0.5% NP40, 0.07%-0.4% NP40, 0.08%-0.3% NP40, 0.09%-0.2% NP40, or 0.1% NP40.

In certain embodiments, lysis buffers include one or more of NP-40, Triton X-100, SDS, guanidine isothiocyante, guanidine hydrochloride or guanidine thiocyanate.

In some embodiments of any of the aspects, the buffer that contacts the cells during permeabilization comprises 1 mM DTT. In some embodiments of any of the aspects, the buffer permeabilization buffer comprises 0.01-1 mM DTT, 0.02-0.9 mM DTT, 0.03-0.8 mM DTT, 0.04-0.7 mM DTT, 0.05-0.6 mM DTT, 0.06-05. mM DTT, 0.07-0.4 mM DTT, 0.08-0.3 mM DTT, 0.09-0.2 mM DTT, or 0.1 mM DTT.

In some embodiments of any of the aspects, the buffer that contacts the cells during permeabilization comprises 1 U/μl RI. In some embodiments of any of the aspects, the buffer permeabilization buffer comprises 0.01-2 mM U/μl RI, 0.02-1.9 U/μl RI, 0.03-1.8 U/μl RI, 0.04-1.7 U/μl RI, 0.05-1.6 U/μl RI, 0.06-1.5 U/μl RI, 0.07-1.4 U/μl RI, 0.08-1.3 U/μl RI, 0.09-1.2 U/μl RI, 0.1-1.1 U/μl RI, 0.2-1.0 U/μl RI, 0.3-0.9 U/μl RI, 0.4-0.8 U/μl RI, 0.5-0.7 U/μl RI, or 0.6 U/μl RI.

In certain embodiments, the buffer that contacts the cells during permeabilization comprises 5-15 mM Tris, 5-15 mM NaCl, 1-5 mM MgCl₂, 0.01%-1% NP40, 0.5-2 mM DTT, and 0.1-2 U/μL RI.

In some embodiments of any of the aspects, the buffer that contacts the cells during permeabilization comprises 0.001%-2% bovine serum albumin (BSA), 0.002%-1.9% BSA, 0.003%-1.8% BSA, 0.004%-1.7% BSA, 0.005%-1.6% BSA, 0.006/6-1.5% BSA, 0.007%-1.4% BSA, 0.008/6-1.3% BSA, 0.009%-1.2% BSA, 0.01%-1.1% BSA, 0.02%-1% BSA, 0.03%-0.9% BSA, 0.04%-0.8%, 0.05%-0.7% BSA, 0.06-0.6% BSA, 0.07-0.5% BSA, 0.08-0.4% BSA, 0.09-0.3% BSA, 0.1-0.2% BSA or 0.15% BSA.

In some embodiments, the concentration of BSA in the permeabilization buffer is at least 0.005%, but less than 1.0%, less than 0.9%, less than 0.8%, less than 0.7%, less than 0.6%, less than 0.5%, less than 0.4%, less than 0.3%, less than 0.2%, 0.1% or less, 0.09% or less, 0.08% or less, 0.07% or less, 0.06% or less, 0.05% or less, 0.04% or less, 0.03% or less, 0.02% or less, 0.01% or less, 0.009% or less, 0.008% or less, 0.007% or less, or 0.006% or less.

In certain embodiments, the buffer that contacts the cells during permeabilization comprises 10 mM Tris, 10 mM NaCl, 2 mM MgCl₂, 0.1% NP40, 1 mM DTT, 0.01% BSA and 1 U/μL RI.

In certain embodiments, the buffer that contacts the cells during permeabilization comprises 10 mM Tris, 10 mM NaCl, 3 mM MgCl₂, 0.1% NP40, 1 mM DTT, 0.01% BSA and 1 U/μL RI.

In certain embodiments, the buffer that contacts the cells during permeabilization does not comprise BSA.

In certain embodiments, the buffer that contacts the cells during permeabilization comprises 10 mM Tris, 10 mM NaCl, 2 mM MgCl₂, 0.1% NP40, 1 mM DTT, and 1 U/μL RI.

In certain embodiments, the buffer that contacts the cells during permeabilization comprises 10 mM Tris, 10 mM NaCl, 3 mM MgCl₂, 0.1% NP40, 1 mM DTT, and 1 U/μL RI.

In some embodiments of any of the aspects, the permeabilization of fixed cells takes place on ice. In some embodiments, the permeabilization of fixed cells takes place at 0° C., at 1° C., at 2° C., at 3° C., at 4° C., at 5° C., at 6° C., at 7° C., at 8° C., at 9° C., at 10° C., at 11° C., at 12° C., at 13° C., at 14° C., at 15° C., at 16° C., at 17° C., at 18° C., at 19° C., or at 20° C.

In some embodiments of any of the aspects, the permeabilization of fixed cells takes place on ice for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes at least 19 minutes, or at least 20 minutes.

In certain embodiments, is a method of fixing and permeabilizing whole cells, comprising (a) fixing the cells by contacting them with a buffer comprising 0.1% FA, 0.04% BSA, and 0.2 U/μL RI and (b) permeabilizing the fixed cells by contacting them with a buffer comprising 10 mM Tris, 10 mM NaCl, 2 mM MgCl₂, 0.1% NP40, 1 mM DTT, and 1 U/μL RI.

In certain embodiments, is a method of fixing and permeabilizing whole cells, comprising (a) fixing the cells by contacting them with a buffer comprising 0.1% FA, 0.04% BSA, and 0.2 U/μL RI and (b) permeabilizing the fixed cells by contacting them with a buffer comprising 10 mM Tris, 10 mM NaCl, 3 mM MgCl₂, 0.1% NP40, 1 mM DTT, and 1 U/μL RI.

The fixation and permeabilization methods described herein provide improved access to mtDNA that provides significant increases in mtDNA sequencing reads and depth of sequencing coverage. These improvements, alone or together with improvements in tagmentation conditions (see below), permit higher resolution when using mtDNA variations as a barcode system for cellular lineage tracing.

### Tagmentation

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1). The term “tagmentation” refers to a step in the Assay for Transposase Accessible Chromatin using sequencing (ATAC-seq) as described. Specifically, a hyperactive Tn5 transposase loaded in vitro with adapters for high-throughput DNA sequencing, can simultaneously fragment and tag a genome with sequencing adapters. In some embodiments, ATAC-seq is performed using ATAC buffer B from 10× Genomics, PN 2000265.

In certain embodiments, tagmentation is performed in an ATAC buffer B comprising 1% glycoldiosgenin (ATAC-GDN) and Tn5 transposase. In certain embodiments, ATACH buffer B is supplemented with 0.1% GDN, with 0.2% GDN, with 0.3% GDN, with 0.4% GDN, with 0.5% GDN, with 0.6% GDN, with 0.7% GDN, with 0.8% GDN, with 0.9% GDN, with 1.0% GDN, with 1.1% GDN, with 1.2% GDN, with 1.3% GDN, with 1.4% GDN, with 1.5% GDN, with 1.6% GDN, with 1.7% GDN, with 1.8% GDN, with 1.9% GDN, or with 2.0% GDN.

In certain embodiments, tagmentation is performed in an ATAC buffer B comprising 1% glycoldiosgenin (ATACH-GDN), 0.1% BSA and Tn5 transposase. In certain embodiments, ATAC buffer B is supplemented with 0.01% BSA, with 0.02% BSA, with 0.04% BSA, with 0.06% BSA, with 0.08% BSA, with 0.1% BSA, with 0.2% BSA, with 0.3% BSA, with 0.4% BSA, with 0.5% BSA, with 0.6% BSA, with 0.7% BSA, with 0.8% BSA, with 0.9% BSA, with 1% BSA, with 1.2% BSA, with 1.4% BSA, with 1.6% BSA, with 1.8% BSA, or with 2.0% BSA.

In some embodiments, GDN is included in the tagmentation buffer at a concentration of 0.1-2% inclusive, e.g., at a concentration of at least 0.1%, at least 0.2%, at least 0.3%, at least 0.4%, at least 0.5%, at least 0.6%, at least 0.7%, at least 0.8%, at least 0.9%, at least 1.0%, at least 1.1%, at least 1.2%, at least 1.3%, at least 1.4%, at least 1.5%, at least 1.6%, at least 1.7%, at least 1.8%, at least 1.9%, or 2.0%.

In certain embodiments, tagmentation takes place at 35° C., at 36° C., at 37° C., at 38° C., at 394° C., at 40° C., at 41° C., at 42° C., at 43° C., at 44° C., at 45° C., at 46° C., at 47° C., at 48° C., at 49° C., or at 50° C.

In some embodiments tagmentation takes place for at least 20 minutes, at least 30 minutes, at least 40 minutes, at least 50 minutes, at least 60 minutes, at least 70 minutes, at least 80 minutes, at least 90 minutes, at least 100 minutes, at least 110 minutes, at least 120 minutes, at least 130 minutes, at least 140 minutes, or at least 150 minutes.

In some embodiments, tagmented cells are processed via Gel Bead-In-EMulsion (GEM) generation and barcoding. In some embodiments, such processes follow 10× Genomics Chromium Next GEM Single Cell Multiome ATAC+ Gene Expression User Manual (version CG000338 Rev A, 10× Genomics) and 10× Genomics Chromium Next GEM Single Cell ATAC Reagent Kits v2 (version CG000496 Rev B, 10× Genomics), which are incorporated herein by reference.

In certain embodiments, Table 1 is an exemplary embodiment of formulations for fixation, permeabilization, and tagmentation.

TABLE 1
Buffers for optimized protocol.

| Component | Concentrations |
|---|---|
| Fixation Buffer | 0.1% formaldehyde in ; 0.2 U/ul RNase |
| | Inhibitor (RI); 0.01% BSA; |
| Permeabilization Buffer | 10 mM Tris, 10 mM NaCl, 3 mM MgCl₂, 0.1% |
| | NP40, 1 mM DTT, 1 U/ul RI; 0.01% BSA; |
| Tagmentation Buffer | ATAC Buffer B (10X Genomics PN-2000193); |
| | 1% GDN; Tn5 |

In certain embodiments, retention and/or availability of mtDNA is at least 2× greater than when fixation, permeabilization, and tagmentation are performed in reference buffers. In some embodiments retention and/or availability of mtDNA is at least 3×, at least 4×, at least 5×, at least 6×, at least 7×, at least 8×, at least 9×, at least 10×, at least 11×, at least 12×, at least 13×, at least 14×, at least 15×, at least 16×, at least 17×, at least 18×, at least 19×, or at least 20× greater than when the reference buffers are used.

In certain embodiments, Table 2 is the formulations for the fixation, permeabilization, and tagmentation reference buffers.

TABLE 2
Reference Buffers

| Component | Concentrations |
|---|---|
| Fixation Buffer | 0.1% formaldehyde in DPBS; 1.0% BSA; 0.2 |
| | U/ul RNase Inhibitor (RI); |
| Permeabilization Buffer | 10 mM Tris, 10 mM NaCl, 3 mM MgCl₂, 0.1% |
| | NP40, 1 mM DTT, 1 U/ul RI; 1% BSA |
| Tagmentation Buffer | ATAC Buffer B (10X Genomics PN-2000193); |
| | Tn5 (No GDN) |

### Sequencing

In certain embodiments, sequencing comprises high-throughput (formerly “next-generation”) technologies to generate sequencing reads. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56(2): 61-77). In certain embodiments, the library members (e.g., genomic DNA, cDNA) can include sequencing adaptors that are compatible with use in, e.g., Illumina's reversible terminator method, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

In certain embodiments, the present invention includes whole genome sequencing. Whole genome sequencing (also known as WGS, full genome sequencing, complete genome sequencing, or entire genome sequencing) is the process of determining the complete DNA sequence of an organism's genome at a single time. This entails sequencing all of an organism's chromosomal DNA as well as DNA contained in the mitochondria and, for plants, in the chloroplast.

In certain embodiments, the present invention includes whole exome sequencing. Exome sequencing, also known as whole exome sequencing (WES), is a genomic technique for sequencing all of the protein-coding genes in a genome (known as the exome) (see, e.g., Ng et al., 2009, Nature volume 461, pages 272-276). It consists of two steps: the first step is to select only the subset of DNA that encodes proteins. These regions are known as exons—humans have about 180,000 exons, constituting about 1% of the human genome, or approximately 30 million base pairs. The second step is to sequence the exonic DNA using any high-throughput DNA sequencing technology. In certain embodiments, whole exome sequencing is used to determine somatic mutations in genes associated with disease (e.g., cancer mutations).

In certain embodiments, targeted sequencing can be used in the present invention (see, e.g., Mantere et al., PLoS Genet 12 e1005816 2016; and Carneiro et al. BMC Genomics, 2012 13:375). Targeted gene sequencing panels are useful tools for analyzing specific mutations in a given sample. Focused panels contain a select set of genes or gene regions that have known or suspected associations with a disease or phenotype under study. In certain embodiments, targeted sequencing is used to detect mutations, e.g., associated with a disease in a subject in need thereof. Targeted sequencing can increase the cost-effectiveness of variant discovery and detection. In certain embodiments, targeting sequencing is used to detect mutations in mtDNA. In certain embodiments, described herein, targeted mitochondrial probes are used to detect mutations in mtDNA.

In certain embodiments, the mitochondrial genome is specifically sequenced in a bulk sample using MitoRCA-seq (see e.g., Ni et al., MitoRCA-seq reveals unbalanced cytosine to thymine transition in Polg mutant mice. Sci Rep. 2015 Jul. 27; 5:12049. doi: 10.1038/srep12049). The method employs rolling circle amplification, which enriches the full-length circular mtDNA by either custom mtDNA-specific primers or a commercial kit, and minimizes the contamination of nuclear encoded mitochondrial DNA (Numts). In certain embodiments, RCA-seq is used to detect low-frequency mtDNA point mutations starting with as little as 1 ng of total DNA.

In certain embodiments, single cell Mito-seq (scMito-seq) is used to sequence the mitochondrial genome in single cells. The method is based on performing rolling circle amplification of mitochondrial genomes in single cells.

In certain embodiments, multiple displacement amplification (MDA) is used to generate a sequencing library (e.g., single cell genome sequencing). Multiple displacement amplification (MDA, is a non-PCR-based isothermal method based on the annealing of random hexamers to denatured DNA, followed by strand-displacement synthesis at constant temperature (Blanco et al. J. Biol. Chem. 1989, 264, 8935-8940). It has been applied to samples with small quantities of genomic DNA, leading to the synthesis of high molecular weight DNA with limited sequence representation bias (Lizardi et al. Nature Genetics 1998, 19, 225-232; Dean et al., Proc. Natl. Acad. Sci. U.S.A. 2002, 99, 5261-5266). As DNA is synthesized by strand displacement, a gradually increasing number of priming events occur, forming a network of hyper-branched DNA structures. The reaction can be catalyzed by enzymes such as the Phi29 DNA polymerase or the large fragment of the Bst DNA polymerase. The Phi29 DNA polymerase possesses a proofreading activity resulting in error rates 100 times lower than Taq polymerase (Lasken et al. Trends Biotech. 2003, 21, 531-535).

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves single cell RNA sequencing (scRNA-seq). In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, cells to be sequenced according to any of the methods herein are lysed under conditions specific to sequencing mitochondrial genomes. In certain embodiments, lysis using mild conditions does not result in sequencing of all of the mitochondrial genomes. In certain embodiments, use of harsher lysing conditions allows for increased sequencing of mitochondrial genomes due to improved lysis of mitochondria. In certain embodiments, lysis buffers include one or more of NP-40, Triton X-100, SDS, guanidine isothiocyante, guanidine hydrochloride or guanidine thiocyanate.

In certain embodiments, the sequencing cost is lower in sequencing mitochondrial genomes because of the size of the mitochondrial genome. The terms “depth” or “coverage” as used herein refers to the number of times a nucleotide is read during the sequencing process. In regards to single cell RNA sequencing, “depth” or “coverage” as used herein refers to the number of mapped reads per cell. Depth in regard to genome sequencing can be calculated from the length of the original genome (G), the number of reads (N), and the average read length (L) as N×L/G. For example, a hypothetical genome with 2,000 base pairs reconstructed from 8 reads with an average length of 500 nucleotides will have 2× redundancy.

The terms “low-pass sequencing” or “shallow sequencing” as used herein refer to a wide range of depths greater than or equal to 0.1× up to 1×. Shallow sequencing can also refer to about 5000 reads per cell (e.g., 1,000 to 10,000 reads per cell).

The term “deep sequencing” as used herein indicates that the total number of reads is many times larger than the length of the sequence under study. The term “deep” as used herein refers to a wide range of depths greater than 1× up to 100×. Deep sequencing can also refer to 100× coverage as compared to shallow sequencing (e.g., 100,000 to 1,000,000 reads per cell).

The term “ultra-deep” as used herein refers to higher coverage (>100-fold), which allows for detection of sequence variants in mixed populations.

As described herein, a nucleic acid strand comprises a unique molecule identifier (UMI) sequence or domain. UMIs are distinct sequences similar to barcodes that are used to distinguish one source DNA molecule from another when DNA molecules are sequenced together. Endogenous UMIs (eUMIs) are UMIs that can be identified within source DNA molecules. In some implementations, eUMIs are located at or near the ends of the source DNA molecule. One or more such unique end positions can alone or in conjunction with other information uniquely identify a source DNA molecule. In some embodiments, the paired-end sequencing for each fragment provides information of single-cell barcode (CellBC) as well as Tn5 cutting sites of the left (start) and right (end), which serve as eUMIs.

### Mitochondria Probes

In certain embodiments, DNA probes are used to contact the ATAC library product for purification and amplification of mtDNA. In some embodiments, four sets of DNA probes are used that cover the whole mitochondrial genome. In some embodiments, each set of DNA probes contains biotinylated oligos that are 120 base pairs long. In some embodiments, the different set of probes are staggered by 30 base pairs. In some embodiments the first set of probes start at the first base pair of the mDNA sequence, the second set of probes start at base pair 30 of the mDNA sequence, the third set of probes start at base pair 60 of the mDNA sequence, and the fourth set of probes start at base pair 90 of the mDNA sequence.

In some embodiments, the DNA probes are 110 base pairs, 111 base pairs, 112 base pairs, 113 base pairs, 114 base pairs, 115 base pairs, 116 base pairs, 117 base pairs, 118 base pairs, 119 base pairs, 120 base pairs, 121 base pairs, 122 base pairs, 123 base pairs, 124 base pairs, 125 base pairs, 126 base pairs, 127 base pairs, 128 base pairs 129 base pairs, 130 base pairs, 131 base pairs, 132 base pairs, 133 base pairs, 134 base pairs, 135 base pairs, 136 base pairs, 137 base pairs, 138 base pairs, 139 base pairs, or 140 base pairs.

In some embodiments, the DNA probes are staggered by 10 base pairs, 11 base pairs, 12 base pairs, 13 base pairs, 14 base pairs, 15 base pairs, 16 base pairs, 17 base pairs, 18 base pairs, 19 base pairs, 20 base pairs, 21 base pairs, 22 base pairs, 23 base pairs, 24 base pairs, 25 base pairs, 26 base pairs, 27 base pairs, 28 base pairs 29 base pairs, 30 base pairs, 31 base pairs, 32 base pairs, 33 base pairs, 34 base pairs, 35 base pairs, 36 base pairs, 37 base pairs, 38 base pairs, 39 base pairs, or 40 base pairs.

In some embodiments, the DNA probes within each set overlap by 2 base pairs, e.g., the first probe covers base pairs 1-120, and the second probe covers base pairs 118-238. In some embodiments, the DNA probes within each set overlap by 0, 1, 2, 3, 4, or 5 base pairs.

### Selecting Mutations

In certain embodiments, somatic mutations occur over time in long lived organisms. In certain embodiments, somatic mutations occur and are propagated over years. Thus, in preferred embodiments, the subjects according to the present invention include higher eukaryotes (e.g., mammals, humans, livestock, cats, dogs, rodents).

As used herein, the term “homoplasmic” refers to a eukaryotic cell whose copies of mitochondrial DNA are all identical or alleles that are identical in all mitochondria. As used herein, the term “homoplasmic” also refers to identical sequencing reads for a specific genomic region.

In certain embodiments, heteroplasmic mitochondrial mutations are selected and used to cluster single cells. As used herein, the term “heteroplasmic” refers to the presence of more than one type of organellar genome (mitochondrial DNA or plastid DNA) within a cell or individual or mutations only occurring in some copies of mitochondrial DNA. Because most eukaryotic cells contain many hundreds of mitochondria with hundreds of copies of mitochondrial DNA, it is common for mutations to affect only some mitochondria, leaving most unaffected. For example, 5% heteroplasmy refers to a mutation being present in 5% of all mitochondrial genomes. As used herein, “heteroplasmic” also refers to the percentage of mutations in terms of number of reads spanning a specific genomic region. For example, if there are 100 sequencing reads across a region, 5% means that this mutation is in 5 out of 100 reads.

In certain embodiments, mitochondrial mutations used for clustering are selected. In certain embodiments, mutations having a certain heteroplasmy are selected. In certain embodiments, heteroplasmy above a threshold is used because these mutations have a higher probability of being passed onto progeny during multiple generations. In certain embodiments, the mutations are 0.1, 0.25, 0.5, 1, 2, 3, 4, 5, 10, 20 or 25% heteroplasmic.

In certain embodiments, mutations are selected in terms of number of reads spanning a specific genomic region. In certain embodiments, mutations are observed in more than 5 reads. For example, if there is only 1 read with the mutation out of 20 reads spanning this region, this mutation can be eliminated as a low confidence mutation. The low confidence mutations can not be “real”. Therefore, in certain embodiments, mutations are selected based on the heteroplasmy in sequencing reads and the number of reads is above a minimum threshold greater than 1 sequencing read having a mutation.

In certain embodiments, heteroplasmy is determined in terms of sequencing reads in all of the single cells analyzed. In certain embodiments, mutations are selected that have greater than 0.5% heteroplasmy. In certain embodiments, mutations are selected based on a conservative threshold and have greater than 5% heteroplasmy.

In certain embodiments, mutations are selected based on mutations detected in mitochondrial genome sequencing reads of a bulk sample obtained from the subject. The bulk sample can be sequenced according to any of the methods for sequencing the mitochondrial genome described above (e.g., DNA-seq, RNA-seq, ATAC-seq or RCA-seq). In certain embodiments, the mitochondrial genome is sequenced directly to determine somatic mutations and not mutations detected due to RNA modifications or reverse transcription errors. In certain embodiments, mutations are selected independently based on detection in the bulk samples and are not further selected based on heteroplasmy. In certain embodiments, the mutations are further selected based on heteroplasmy and mutations are selected from the bulk sample that are greater than 0.5% heteroplasmy. In certain embodiments, the mutations detected in the bulk sample are observed in greater than 1 sequencing read.

In certain embodiments, mutations are selected based on a base quality score. In certain embodiments, the detected mutations have a Phred quality score greater than 20. A Phred quality score is a measure of the quality of the identification of the nucleobases generated by automated DNA sequencing (see, e.g., Ewing et al., (1998). “Base-calling of automated sequencer traces using phred. I. Accuracy assessment”. Genome Research. 8 (3): 175-185; and Ewing and Green (1998). “Base-calling of automated sequencer traces using phred. II. Error probabilities”. Genome Research. 8 (3): 186-194). It was originally developed for Phred base calling to help in the automation of DNA sequencing in the Human Genome Project. Phred quality scores are assigned to each nucleotide base call in automated sequencer traces. Phred quality scores have become widely accepted to characterize the quality of DNA sequences, and can be used to compare the efficacy of different sequencing methods. Perhaps the most important use of Phred quality scores is the automatic determination of accurate, quality-based consensus sequences.

The method can further comprise excluding RNA modifications, RNA transcription errors and/or RNA sequencing errors from the mutations detected. The RNA modifications can comprise previously identified RNA modifications. These include RNA modifications known in the art and modifications identified by sequencing mitochondrial genomes and comparing the sequences to mitochondrial transcripts. In certain embodiments, RNA modifications, RNA transcription errors and/or RNA sequencing errors are determined by comparing the mutations detected by scRNA-seq to mutations detected by DNA-seq, ATAC-seq or RCA-seq in a bulk sample from the subject.

### Mutation Calling

In certain embodiments, the method comprises selecting mtDNA mutations that are uniquely mapped to the mitochondrial genome. In certain embodiments, the mutation is detected in the sequences in strands sequenced in both directions, e.g., read 1 and read 2. In other embodiments, the mutation is detected in only one read.

In certain embodiments, the mutation base has a sequence quality score of 30 or greater. In certain embodiments, the eUMI group size is 2, e.g., if the mutation base was read in both directions. In certain embodiments, the eUMI group size is 3, e.g., if the mutation base was read in only one direction. In certain embodiments, the eUMI consensus score is at least 0.75%. In certain embodiments, the eUMI consensus score is at least 100%. In certain embodiments, mutations are selected if the mutation is shown in two or more than two cells and with a max allele count of two or more than two alleles in one single cell. In certain embodiments, mutations are discarded if they are detected as homoplasmic mutations that appear in all single cells. In certain embodiments, mutation are discarded if they are detected as heteroplasmic mutations that only appear in one single cell.

### Determining a Lineage or Clonal Structure

As used herein the terms “lineage” or “clonal structure” refer to the relationship between any two or more cells. As used herein, the term “cell lineage” refers to the developmental path by which a fertilized egg gives rise to the cells of a multicellular organism or the developmental history of a tissue or organ. As discussed herein in regard generally to cells in multicellular organisms, and particularly in cells with high turnover, e.g., hematopoietic cells, cells of the gut lining, etc., variations in cell lineage traced by genetic composition during the life of the organism or from generation to generation of such high turnover cells, can illuminate factors important to cell status. Cell lineages can be displayed figuratively or schematically on a lineage map.

In this context, a “clone” is a group of cells that share a common ancestry, meaning they are derived from the same cell. In certain embodiments, new mutations arise over time in a clonal population giving rise to sub-clonal populations of cells. The assessment or assignment of clonal structure in a given population permits assessment of clonal contributions of clones and sub-clones in the population, for example in hematopoietic cells or in a tumor. In certain embodiments, the clonal structure is determined before and after a treatment.

In certain embodiments, such as in multicellular organisms, the progeny of single dividing cells cannot be followed and a cell lineage or clonal structure is inferred retrospectively (e.g., after cell division has already occurred). The technology described herein provides for improved methods of inferring a cell lineage or clonal structure by detecting somatic mutations, specifically somatic mutations that occur in the mitochondrial genome.

Determination of somatic mutations (e.g., including mitochondrial mutations) allows cells derived from a tissue or tumor to be clustered based on the mutations. In certain embodiments, the method further comprises detecting mutations in the nuclear genome and clustering the cells based on the presence of the mitochondrial and nuclear genome mutations in the single cells. In certain embodiments, the method comprises sequencing the nuclear genome in single cells obtained from the subject according to a sequencing method described herein (e.g., whole genome, whole exome sequencing). The clustering provides for related cells.

As used herein, the term “clustering” or “cluster analysis” refers to the task of grouping a set of objects (e.g., cells) in such a way that objects in the same group (referred to as a cluster) are more similar (in some sense) to each other than to those in other groups (clusters). Clustering is a main task of exploratory data mining, and a common technique for statistical data analysis, used in many fields, including machine learning, pattern recognition, image analysis, information retrieval, bioinformatics, data compression, and computer graphics.

Cluster analysis itself is not one specific algorithm, but the general task to be solved. It can be achieved by various algorithms that differ significantly in their approach to what constitutes a cluster and how to efficiently find them. Popular notions of clusters include groups with small distances between cluster members, dense areas of the data space, intervals or particular statistical distributions. Clustering can therefore be formulated as a multi-objective optimization problem. The appropriate clustering algorithm and parameter settings (including parameters such as the distance function to use, a density threshold or the number of expected clusters) depend on the individual data set and intended use of the results. Cluster analysis as such is not an automatic task, but an iterative process of knowledge discovery or interactive multi-objective optimization that involves trial and failure. It is often necessary to modify data preprocessing and model parameters until the result achieves the desired properties. In certain embodiments, clustering is performed based on somatic mutations present in single cells. In certain embodiments, clustering is performed based on the transcriptomes of single cells.

Clustering can employ different algorithms to generate cluster models. Typical cluster models include:

Connectivity models: for example, hierarchical clustering builds models based on distance connectivity.

Centroid models: for example, the k-means algorithm represents each cluster by a single mean vector.

Distribution models: clusters are modeled using statistical distributions, such as multivariate normal distributions used by the expectation-maximization algorithm.

Density models: for example, DBSCAN and OPTICS defines clusters as connected dense regions in the data space.

Subspace models: in biclustering (also known as co-clustering or two-mode-clustering), clusters are modeled with both cluster members and relevant attributes.

Group models: some algorithms do not provide a refined model for their results and just provide the grouping information.

Graph-based models: a clique, that is, a subset of nodes in a graph such that every two nodes in the subset are connected by an edge can be considered as a prototypical form of cluster. Relaxations of the complete connectivity requirement (a fraction of the edges can be missing) are known as quasi-cliques, as in the HCS clustering algorithm.

Neural models: the most well-known unsupervised neural network is the self-organizing map and these models can usually be characterized as similar to one or more of the above models, and including subspace models when neural networks implement a form of Principal Component Analysis or Independent Component Analysis.

A “clustering” is essentially a set of such clusters, usually containing all objects in the data set. Additionally, it can specify the relationship of the clusters to each other, for example, a hierarchy of clusters embedded in each other. Clusterings can be roughly distinguished as:

Hard clustering: each object belongs to a cluster or not.

Soft clustering (also: fuzzy clustering): each object belongs to each cluster to a certain degree (for example, a likelihood of belonging to the cluster)

There are also finer distinctions possible, for example:

Strict partitioning clustering: each object belongs to exactly one cluster.

Strict partitioning clustering with outliers: objects can also belong to no cluster, and are considered outliers.

Overlapping clustering (also: alternative clustering, multi-view clustering): objects can belong to more than one cluster; usually involving hard clusters.

Hierarchical clustering: objects that belong to a child cluster also belong to the parent cluster.

Subspace clustering: while an overlapping clustering, within a uniquely defined subspace, clusters are not expected to overlap.

In certain embodiments, single cells are clustered by hierarchical clustering using somatic mutations.

### Cell States

In certain embodiments, the cell states of the clusters are determined. Thus, cell states can be mapped to specific lineage or clonal structures. As used herein, the term “cell state” includes, but is not limited to the gene expression, epigenetic configuration, and/or nuclear structure of single cells. The cell state can include a differentially expressed gene, differentially expressed gene signature, or differentially accessible chromatin loci, as non-limiting examples.

In certain embodiments, the cell state is determined by analyzing the sequencing data generated for determining somatic mutations (e.g., scRNA-seq, scATAC-seq). Single cell RNA sequencing permits the detection of mitochondrial genome mutations in the transcribed mitochondrial RNA. Mitochondrial RNA is polyadenylated and can be captured by methods that use poly T to reverse transcribe and/or capture mRNA. Single cell ATAC-seq is a high-throughput sequencing technique that identifies open chromatin. Depending on the cell type, ATAC-seq samples can contain ~20-80% mitochondrial sequencing reads which are normally removed as it increases the cost of sequencing. Where ATAC-seq samples are, in some cases, mtDNA heavy, it is well suited to the methods described herein that utilize mtDNA for lineage tracing. In certain embodiments, single cells are analyzed in separate reaction vessels to preserve the ability to analyze the single cells. Analysis can include proteomic and genomic analysis on the single cells.

In certain embodiments, heritable cell states are identified. Heritable cell states can be cell states that are passed down through a lineage (e.g., specific gene signatures shared by cells in a lineage). In certain embodiments, the establishment of a cell state along a lineage is identified (e.g., when a cell state is established).

### Use of Signature Genes

As used herein a “signature” encompasses any gene or genes, protein or proteins, or epigenetic element(s) whose expression profile or whose occurrence is associated with a specific cell type, subtype, or cell state of a specific cell type or subtype within a population of cells. Levels of expression or activity or prevalence can be compared between different cells in order to characterize or identify for instance signatures specific for cell (sub)populations. Increased or decreased expression or activity or prevalence of signature genes can be compared between different cells in order to characterize or identify for instance specific cell (sub)populations. The detection of a signature in single cells can be used to identify and quantitate for instance specific cell (sub)populations. A signature can include a gene or genes, protein or proteins, or epigenetic element(s) whose expression or occurrence is specific to a cell (sub)population, such that expression or occurrence is exclusive to the cell (sub)population. A gene signature as used herein, can thus refer to any set of up- and down-regulated genes that are representative of a cell type or subtype. A gene signature as used herein, can also refer to any set of up- and down-regulated genes between different cells or cell (sub)populations derived from a gene-expression profile. For example, a gene signature can comprise a list of genes differentially expressed in a distinction of interest.

The signature as defined herein (be it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature can be indicative of cells within a population of cells in vivo. The signature can also be used to suggest, for instance, particular therapies, or to follow up treatment, or to suggest ways to modulate immune systems. The signatures of the present invention can be discovered by analysis of expression profiles of single-cells within a population of cells from isolated samples (e.g. tumor samples), thus allowing the discovery of novel cell subtypes or cell states that were previously invisible or unrecognized. The presence of subtypes or cell states can be determined by subtype specific or cell state specific signatures. The presence of these specific cell (sub)types or cell states can be determined by applying the signature genes to bulk sequencing data in a sample. Not wishing to be bound by theory, the signatures of the present invention can be microenvironment specific, such as their expression in a particular spatio-temporal context. Not wishing to be bound by theory, signatures as discussed herein are specific to a particular pathological context. Not wishing to be bound by theory, a combination of cell subtypes having a particular signature can indicate an outcome. Not wishing to be bound by theory, the signatures can be used to deconvolute the network of cells present in a particular pathological condition. Not wishing to be bound by theory, the presence of specific cells and cell subtypes can be indicative of a particular response to treatment, such as increased or decreased susceptibility to treatment. The signature can indicate the presence of one particular cell type. In one embodiment, the novel signatures are used to detect multiple cell states or hierarchies that occur in subpopulations of cancer cells that are linked to particular a pathological condition (e.g. cancer grade), or linked to a particular outcome or progression of a disease (e.g. metastasis), or linked to a particular response to treatment of a disease.

The signature according to certain embodiments of the present invention can comprise or consist of one or more genes, proteins and/or epigenetic elements, such as for instance 1, 2, 3, 4, 5, 6, 7, 8, 9, 10 or more. In certain embodiments, the signature can comprise or consist of two or more genes, proteins and/or epigenetic elements, such as for instance 2, 3, 4, 5, 6, 7, 8, 9, 10 or more. In certain embodiments, the signature can comprise or consist of three or more genes, proteins and/or epigenetic elements, such as for instance 3, 4, 5, 6, 7, 8, 9, 10 or more. In certain embodiments, the signature can comprise or consist of four or more genes, proteins and/or epigenetic elements, such as for instance 4, 5, 6, 7, 8, 9, 10 or more. In certain embodiments, the signature can comprise or consist of five or more genes, proteins and/or epigenetic elements, such as for instance 5, 6, 7, 8, 9, 10 or more. In certain embodiments, the signature can comprise or consist of six or more genes, proteins and/or epigenetic elements, such as for instance 6, 7, 8, 9, 10 or more. In certain embodiments, the signature can comprise or consist of seven or more genes, proteins and/or epigenetic elements, such as for instance 7, 8, 9, 10 or more. In certain embodiments, the signature can comprise or consist of eight or more genes, proteins and/or epigenetic elements, such as for instance 8, 9, 10 or more. In certain embodiments, the signature can comprise or consist of nine or more genes, proteins and/or epigenetic elements, such as for instance 9, 10 or more. In certain embodiments, the signature can comprise or consist of ten or more genes, proteins and/or epigenetic elements, such as for instance 10, 11, 12, 13, 14, 15, or more. It is to be understood that a signature according to the invention can for instance also include genes or proteins as well as epigenetic elements combined.

In certain embodiments, a signature is characterized as being specific for a particular tumor cell or tumor cell (sub)population if it is upregulated or only present, detected or detectable in that particular tumor cell or tumor cell (sub)population, or alternatively is downregulated or only absent, or undetectable in that particular tumor cell or tumor cell (sub)population. In this context, a signature consists of one or more differentially expressed genes/proteins or differential epigenetic elements when comparing different cells or cell (sub)populations, including comparing different tumor cells or tumor cell (sub)populations, as well as comparing tumor cells or tumor cell (sub)populations with non-tumor cells or non-tumor cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression can be determined based on common statistical tests, as is known in the art.

As discussed herein, differentially expressed genes/proteins, or differential epigenetic elements can be differentially expressed on a single cell level, or can be differentially expressed on a cell population level. Preferably, the differentially expressed genes/proteins or epigenetic elements as discussed herein, such as those constituting the gene signatures as discussed herein, when used in reference to a cell population, refer to genes that are differentially expressed in all or substantially all cells of the population (such as at least 80%, preferably at least 90%, such as at least 95% of the individual cells). This allows one to define a particular subpopulation of tumor cells. As referred to herein, a “subpopulation” of cells preferably refers to a particular subset of cells of a particular cell type which can be distinguished or are uniquely identifiable and set apart from other cells of this cell type. The cell subpopulation can be phenotypically characterized, and is preferably characterized by the signature as discussed herein. A cell (sub)population as referred to herein can constitute a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

Signatures can be functionally validated as being uniquely associated with a particular immune responder phenotype. Induction or suppression of a particular signature can consequentially be associated with or causally drive a particular immune responder phenotype.

Various aspects and embodiments of the invention can involve analyzing gene signatures, protein signature, and/or other genetic or epigenetic signature based on single cell analyses (e.g. single cell RNA sequencing) or alternatively based on cell population analyses, as is defined herein elsewhere.

In further aspects, the invention relates to gene signatures, protein signature, and/or other genetic or epigenetic signature of particular tumor cell subpopulations, as defined herein elsewhere. The invention hereto also further relates to particular tumor cell subpopulations, which can be identified based on the methods according to the invention as discussed herein; as well as methods to obtain such cell (sub)populations and screening methods to identify agents capable of inducing or suppressing particular tumor cell (sub)populations.

The invention further relates to various uses of the gene signatures, protein signature, and/or other genetic or epigenetic signature as defined herein, as well as various uses of the tumor cells or tumor cell (sub)populations as defined herein. Particular advantageous uses include methods for identifying agents capable of inducing or suppressing particular tumor cell (sub)populations based on the gene signatures, protein signature, and/or other genetic or epigenetic signature as defined herein. The invention further relates to agents capable of inducing or suppressing particular tumor cell (sub)populations based on the gene signatures, protein signature, and/or other genetic or epigenetic signature as defined herein, as well as their use for modulating, such as inducing or repressing, a particular gene signature, protein signature, and/or other genetic or epigenetic signature. In one embodiment, genes in one population of cells can be activated or suppressed in order to affect the cells of another population. In related aspects, modulating, such as inducing or repressing, a particular a particular gene signature, protein signature, and/or other genetic or epigenetic signature can modify overall tumor composition, such as tumor cell composition, such as tumor cell subpopulation composition or distribution, or functionality.

The signature genes of the present invention can be discovered by analysis of expression profiles of single-cells within a population of cells from freshly isolated tumors, thus allowing the discovery of novel cell subtypes that were previously invisible in a population of cells within a tumor. The presence of subtypes can be determined by subtype specific signature genes. The presence of these specific cell types can be determined by applying the signature genes to bulk sequencing data in a patient tumor. Not wishing to be bound by theory, a tumor is a conglomeration of many cells that make up a tumor microenvironment, whereby the cells communicate and affect each other in specific ways. As such, specific cell types within this microenvironment can express signature genes specific for this microenvironment. Not wishing to be bound by theory, the signature genes of the present invention can be microenvironment specific, such as their expression in a tumor. Not wishing to be bound by theory, signature genes determined in single cells that originated in a tumor are specific to other tumors. Not wishing to be bound by theory, a combination of cell subtypes in a tumor can indicate an outcome. Not wishing to be bound by theory, the signature genes can be used to deconvolute the network of cells present in a tumor based on comparing them to data from bulk analysis of a tumor sample. Not wishing to be bound by theory, the presence of specific cells and cell subtypes can be indicative of tumor growth, invasiveness and resistance to treatment. The signature gene can indicate the presence of one particular cell type. In one embodiment, the signature genes can indicate that tumor infiltrating T-cells are present. The presence of cell types within a tumor can indicate that the tumor will be resistant to a treatment. In one embodiment, the signature genes of the present invention are applied to bulk sequencing data from a tumor sample obtained from a subject, such that information relating to disease outcome and personalized treatments is determined. In one embodiment, the novel signature genes are used to detect multiple cell states that occur in a subpopulation of tumor cells that are linked to resistance to targeted therapies and progressive tumor growth.

In one embodiment, the signature genes are detected by immunofluorescence, immunohistochemistry, fluorescence activated cell sorting (FACS), mass cytometry (CyTOF), Drop-seq, RNA-seq, scRNA-seq, InDrop, single cell qPCR, MERFISH (multiplex (in situ) RNA FISH) and/or by in situ hybridization. Other methods including absorbance assays and colorimetric assays are known in the art and can be used herein.

In one embodiment, tumor cells are stained for sub-clonal cell type specific signature genes. In one embodiment, the cells are fixed. In another embodiment, the cells are formalin fixed and paraffin embedded. Not wishing to be bound by theory, the presence of the cell subtypes in a tumor can indicate outcome and personalized treatments. Not wishing to be bound by theory, the cell subtypes can be quantitated in a section of a tumor and the number of cells can indicate an outcome and personalized treatment.

### Lineages and Clonal Populations in Tissues

In certain embodiments, the single cells comprise related cell types. The related cell types can be from a tissue. In certain embodiments, lineage or clonal structures are determined for specific tissues. The tissue can be associated with a disease state. The disease can be a degenerative disease. The tissue can be healthy tissue. Thus, healthy tissue can be studied to understand a disease state. The tissue can be diseased tissue. Thus, diseased tissue can be studied to understand a disease state.

As used herein, the term “clonal population” refers to a cell lineage, derived from a single cell.

The present invention provides for a method of identifying changes in clonal populations having a cell state between healthy and diseased tissue comprising determining clonal populations of cells having a cell state in healthy and diseased cells as described herein, and comparing the clonal populations. Thus, clonal populations are determined in healthy and diseased tissues. The cell states in the clonal populations can be determined. The tissues can be obtained from the same subject. The cell states are then determined for the clonal populations. Clonal populations shared between the diseased and healthy tissues, as well as clonal populations differentially present or absent between the diseased and healthy tissues can be determined. The present invention allows for improved determination of clonal populations and thus can provide for novel therapeutic targets present in specific populations.

The disease can be selected from the group consisting of autoimmune disease, bone marrow failure, hematological conditions, aplastic anemia, beta-thalassemia, diabetes, motor neuron disease, Parkinson's disease, spinal cord injury, muscular dystrophy, kidney disease, liver disease, multiple sclerosis, congestive heart failure, head trauma, lung disease, psoriasis, liver cirrhosis, vision loss, cystic fibrosis, hepatitis C virus, human immunodeficiency virus, inflammatory bowel disease (IBD), and any disorder associated with tissue degeneration.

As used throughout the present specification, the terms “autoimmune disease” or “autoimmune disorder” used interchangeably refer to a diseases or disorders caused by an immune response against a self-tissue or tissue component (self-antigen) and include a self-antibody response and/or cell-mediated response. The terms encompass organ-specific autoimmune diseases, in which an autoimmune response is directed against a single tissue, as well as non-organ specific autoimmune diseases, in which an autoimmune response is directed against a component present in two or more, several or many organs throughout the body.

Non-limiting examples of autoimmune diseases include but are not limited to acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behçet's disease; coeliac disease; dermatomyositis; diabetes mellitus type I; Goodpasture's syndrome; Graves' disease; Guillain-Barre syndrome (GBS); Hashimoto's disease; idiopathic thrombocytopenic purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus, pernicious anaemia; polyarteritis nodosa; polymyositis; primary biliary cirrhosis; primary myoxedema; psoriasis; rheumatic fever; rheumatoid arthritis; Reiter's syndrome; scleroderma; Sjögren's syndrome; systemic lupus erythematosus; Takayasu's arteritis; temporal arteritis; vitiligo; warm autoimmune hemolytic anemia; or Wegener's granulomatosis.

In certain embodiments, tissue specific mitochondrial mutations are determined for a subject. The tissue specific mitochondrial mutations can be used to better characterize tissues in healthy tissues and diseased tissue. In certain embodiments, tissue specific mutations can be used to determine the cell origin of metastatic cancer of unknown primary origin.

### Clonal Populations in Tumors

In another aspect, the present invention provides for a method of detecting clonal populations of cells in a tumor sample obtained from a subject in need thereof. In certain embodiments, clonal populations of cells are identified based on the presence of the mitochondrial mutations and somatic mutations associated with the cancer in the single cells.

Somatic mutations associated with cancer can include mutations associated with prognosis, treatment or resistance to treatment. Mutations associated across the spectrum of human cancer types have been identified (e.g., Hodis E. et al., Cell. (2012) July 20; 150(2):251-63; and Vogelstein, et al., Science (2013) March 29: Vol. 339, Issue 6127, pp. 1546-1558). A directory of cancer mutations, including gene specific mutations can be found at cancer.sanger.ac.uk/cosmic, the Catalogue of Somatic Mutations in Cancer (COSMIC) (Forbes, et al.; COSMIC: somatic cancer genetics at high-resolution. Nucleic Acids Res 2017; 45 (D1): D777-D783. doi: 10.1093/nar/gkw1121) and www.mycancergenome.org. In certain embodiments, any of these known mutations can be detected depending on the cancer type.

The tumor sample can be obtained before a cancer treatment. The method can further comprise obtaining a sample after treatment and comparing the presence of clonal populations before and after treatment, wherein clonal populations of cells sensitive and resistant to the treatment are identified. The method can comprise determining mutations and subclonal populations on at least one time point after administration of the therapy. The at least one time point can be a week, a month, a year, two years, three years, or five years after initiation of a therapy. The time point can be after a relapse in the disease is detected. Relapse can be any recurrence of symptoms of a disease after a period of improvement. Time points can be taken at any point after the initial treatment of the disease and includes time points following a change to the treatment or after the treatment has been completed.

The cancer treatment can be selected from the group consisting of chemotherapy, radiation therapy, immunotherapy, targeted therapy and a combination thereof.

The therapeutic agent is for example, a chemotherapeutic or biotherapeutic agent, radiation, or immunotherapy. Any suitable therapeutic treatment for a particular cancer can be administered. Examples of chemotherapeutic and biotherapeutic agents include, but are not limited to an angiogenesis inhibitor, such as angiostatin Kl-3, DL-a-Difluoromethyl-ornithine, endostatin, fumagillin, genistein, minocycline, staurosporine, and thalidomide; a DNA intercalator/cross-linker, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; a DNA synthesis inhibitor, such as (±)-Amethopterin (Methotrexate), 3-Amino-1,2,4-benzotriazine 1,4-di oxide, Aminopterin, Cytosine β-D-arabinofuranoside, 5-Fluoro-5′-deoxyuridine, 5-Fluorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (−)-Deguelin, 5,6-Dichlorobenzimidazole I-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, and Troglitazone; a microtubule inhibitor, such as Colchicine, docetaxel, Dolastatin 15, Nocodazole, Paclitaxel, Podophyllotoxin, Rhizoxin, Vinblastine, Vincristine, Vindesine, and Vinorelbine (Navelbine); and an unclassified antitumor agent, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-a, Rapamycin, Sex hormone-binding globulin, Thapsigargin, Vismodegib (Erivedge™), and Urinary trypsin inhibitor fragment (Bikunin). The antitumor agent can be a monoclonal antibody or antibody drug conjugate, such as rituximab (Rituxan®), alemtuzumab (Campath®), Ipilimumab (Yervoy®), Bevacizumab (Avastin®), Cetuximab (Erbitux®), panitumumab (Vectibix®), and trastuzumab (Herceptin®), Tositumomab and 1311-tositumomab (Bexxar®), ibritumomab tiuxetan (Zevalin®), brentuximab vedotin (Adcetris®), siltuximab (Sylvant™), pembrolizumab (Keytruda®), ofatumumab (Arzerra®), obinutuzumab (Gazyva™), 90Y-ibritumomab tiuxetan, 1311-tositumomab, pertuzumab (Perjeta™), ado-trastuzumab emtansine (Kadcyla™), Denosumab (Xgeva®), and Ramucirumab (Cyramza™). The antitumor agent can be a small molecule kinase inhibitor, such as Vemurafenib (Zelboraf®), imatinib mesylate (Gleevec®), erlotinib (Tarceva®), gefitinib (Iressa®), lapatinib (Tykerb®), regorafenib (Stivarga®), sunitinib (Sutent®), sorafenib (Nexavar®), pazopanib (Votrient®), axitinib (Inlyta®), dasatinib (Sprycel®), nilotinib (Tasigna®), bosutinib (Bosulif®), ibrutinib (Imbruvica™), idelalisib (Zydelig®), crizotinib (Xalkori®), afatinib dimaleate (Gilotrif®), ceritinib (LDK378/Zykadia), trametinib (Mekinist®), dabrafenib (Tafinlar®), Cabozantinib (Cometriq™), vandetanib (Caprelsa®), The antitumor agent can be a proteosome inhibitor, such as bortezomib (Velcade®) and carfilzomib (Kyprolis®). The antitumor agent can be a cytokine such as interferons (INFs), interleukins (ILs), or hematopoietic growth factors. The antitumor agent can be INF-a, IL-2, Aldesleukin IL-2, Erythropoietin, Granulocyte-macrophage colony-stimulating factor (GM-CSF) or granulocyte colony-stimulating factor. The antitumor agent can be a targeted therapy such as toremifene (Fareston®), fulvestrant (Faslodex®), anastrozole (Arimidex®), exemestane (Aromasin®), letrozole (Femara®), ziv-aflibercept (Zaltrap®), Alitretinoin (Panretin®), temsirolimus (Torisel®), Tretinoin (Vesanoid®), denileukin diftitox (Ontak®), vorinostat (Zolinza®), romidepsin (Istodax®), bexarotene (Targretin®), pralatrexate (Folotyn®), lenaliomide (Revlimid®), belinostat (Beleodaq™), lenaliomide (Revlimid®), pomalidomide (Pomalyst®), Cabazitaxel (Jevtana®), enzalutamide (Xtandi®), abiraterone acetate (Zytiga®), radium 223 chloride (Xofigo®), or everolimus (Afinitor®). The antitumor agent can be a checkpoint inhibitor such as an inhibitor of the programmed death-1 (PD-1) pathway, for example an anti-PDl antibody (Nivolumab). The inhibitor can be an anti-cytotoxic T-lymphocyte-associated antigen (CTLA-4) antibody. The inhibitor can target another member of the CD28 CTLA4 Ig superfamily such as BTLA, LAG3, ICOS, PDL1 or KIR. A checkpoint inhibitor can target a member of the TNFR superfamily such as CD40, OX40, CD 137, GITR, CD27 or TIM-3. Additionally, the antitumor agent can be an epigenetic targeted drug such as HDAC inhibitors, kinase inhibitors, DNA methyltransferase inhibitors, histone demethylase inhibitors, or histone methylation inhibitors. The epigenetic drugs can be Azacitidine (Vidaza), Decitabine (Dacogen), Vorinostat (Zolinza), Romidepsin (Istodax), or Ruxolitinib (Jakafi).

The immunotherapy can be adoptive cell transfer therapy. As used herein, “ACT”, “adoptive cell therapy” and “adoptive cell transfer” can be used interchangeably. In certain embodiments, Adoptive cell therapy (ACT) can refer to the transfer of cells to a patient with the goal of transferring the functionality and characteristics into the new host by engraftment of the cells. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis. Additionally, chimeric antigen receptors (CARs) can be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

The immunotherapy can be an inhibitor of check point protein. Specific check point inhibitors include, but are not limited to anti-CTLA4 antibodies (e.g., Ipilimumab), anti-PD-1 antibodies (e.g., Nivolumab, Pembrolizumab), and anti-PD-L1 antibodies (e.g., Atezolizumab).

### Screening

In another aspect, the present invention provides for a method of identifying a cancer therapeutic target. In certain embodiments, clonal populations of cells in a tumor sample are detected. Differential cell states can be identified (e.g., transcriptional or chromatin) between the clonal populations. Cell states present in resistant clonal populations can be determined by identifying clonal populations after treatment, preferably before and after treatment. The cell states identified between clonal populations can be used to identify a therapeutic target. The cell state can be a differentially expressed gene, differentially expressed gene signature, or differentially accessible chromatin loci. The current method provides for improved determination of clonal populations of cells, thus differential expression or cell states between clonal populations can be determined. Previous methods can not identify a therapeutic target.

In another aspect, the present invention provides for a method of screening for a cancer treatment. A tumor sample can be obtained from a subject in need thereof. The tumor sample can be grown ex vivo. The tumor sample can be used to generate a patient derived xenograft. Patient derived xenografts (PDX) are models of cancer, where tissue or cells from a patient's tumor are implanted into an immunodeficient mouse. PDX models are used to create an environment that resembles the natural growth of cancer, for the study of cancer progression and treatment. Humanized-xenograft models are created by co-engrafting the patient tumor fragment and peripheral blood or bone marrow cells into a NOD/SCID mouse (Siolas D, Hannon G J (September 2013). “Patient-derived tumor xenografts: transforming clinical samples into mouse models”. Cancer Research (Perspective). 73 (17): 5315-9). The co-engraftment allows for reconstitution of the murine immune system enabling researchers to study the interactions between xenogenic human stroma and tumor environments in cancer progression and metastasis (Talmadge J E, Singh R K, Fidler U, Raz A (March 2007). “Murine models to evaluate novel and conventional therapeutic strategies for cancer”. The American Journal of Pathology (Review). 170 (3): 793-804). Clonal populations can be detected in the tumor sample. The tumor sample or mouse model can be treated according to the standard of care for the cancer (e.g., targeting BCR-ABL in CML). The effect of the treatment on the clonal populations can be determined. In one embodiment, it can be determined that the treatment will be effective for the subject's tumor. The effect of the treatment on the clonal populations can be determined and differentially expressed genes between resistant and sensitive clonal populations can be used to determine therapeutic targets. Determining the effects on clonal populations can be determined by measuring expression of a gene signature associated with the clonal populations.

In certain embodiments, tumor clonal structures are measured, cancer therapeutic targets are identified, and/or therapeutics are screened for a specific cancer. The cancer can include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer can include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

### Selecting Cell Types

In certain embodiments, the cells obtained from a subject are selected for a cell type. In certain embodiments, stem and progenitor cells are selected. In certain embodiments, progenitor cells specific for generating a specific tissue are identified. In certain embodiments, cells along a lineage specific for generating a specific tissue are identified. In certain embodiments, CD34+ hematopoietic stem and progenitor cells can be selected (e.g., to study blood diseases).

In certain embodiments, the method further comprises determining a lineage and/or clonal structure for single cells from two or more tissues and identifying tissue specific mitochondrial mutations for the subject. In certain embodiments, the related cell types are from a tumor sample. In certain embodiments, peripheral blood mononuclear cells (PBMCs) and/or bone marrow mononuclear cells (BMMCs) are selected. The PBMCs and/or BMMCs can be selected before and after stem cell transplantation in a subject.

In certain embodiments, lineages or clonal structures for populations of immune cells can be determined (e.g., T cells specific for an antigen).

The term “immune cell” generally encompasses any cell derived from a hematopoietic stem cell that plays a role in the immune response. The term is intended to encompass immune cells both of the innate or adaptive immune system. The immune cell as referred to herein can be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4+, CD8+, effector Th, memory Th, regulatory Th, CD4+/CD8+ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

The present invention provides a novel analytic framework, methods and systems that are widely applicable across diseases, and specifically different types of cancer. The present invention provides for the detection and grouping of subclonal populations of cells or disease causing entities based upon mitochondrial mutations present in each cell or disease causing entity. The subclones can be present in less than 10%, less than 5%, less than 1%, less than 0.1%, less than 0.01%, less than 0.001% or less than 0.0001% of the diseased cells or malignant cells. The disease can be any disease where drug resistance mutations occur or where clonal evolution occurs.

In one aspect, the present invention provides a method of individualized or personalized treatment for a disease undergoing clonal evolution and for preventing relapse after treatment in a patient in need thereof comprising: determining mutations present in a disease cell fraction from the patient before and/or after administration of a therapy; determining subclonal populations within the disease cell fraction; selecting at least one subclonal population to treat.

### Exemplary Embodiments

The present invention can further be described in the following numbered paragraphs:

Embodiment 1: A method of fixing and permeabilizing whole cells, the method comprising:
a) fixing the cells by contacting them with a buffer comprising 0.01%-1% formaldehyde in DPBS, 0.001/6-2% bovine serum albumin (BSA) and 0.1-1 U/ul RNase Inhibitor (RI); and
b) permeabilizing the fixed cells by contacting them with a buffer comprising 10 mM Tris, 10 mM NaCl, 3 mM MgCl₂, 0.01-1% NP40, 0.5-2 mM DTT, 0%-2% BSA and 0.1-2 U/ul RI.

Embodiment 2: A method of fixing and permeabilizing whole cells, the method comprising:
a) fixing the cells by contacting them with a buffer comprising 0.1% formaldehyde in DPBS, 0.01% bovine serum albumin (BSA) and 0.2 U/ul RNase Inhibitor (RI); and
b) permeabilizing the fixed cells by contacting them with a buffer comprising 10 mM Tris, 10 mM NaCl, 2 mM MgCl₂, 0.1% NP40, 1 mM DTT, 0.01% BSA and 1 U/ul RI.

Embodiment 3: A method of fixing and permeabilizing whole cells, the method comprising:
a) fixing the cells by contacting them with a buffer comprising 0.1% formaldehyde in DPBS, 0.04% bovine serum albumin (BSA) and 0.2 U/ul RNase Inhibitor (RI); and
b) permeabilizing the fixed cells by contacting them with a buffer comprising 10 mM Tris, 10 mM NaCl, 2 mM MgCl₂, 0.1% NP40, 1 mM DTT and 1 U/ul RI.

Embodiment 4: The method of embodiment 1, wherein the cells are in a tissue preparation.

Embodiment 5: The method of embodiment 4, wherein the cells are in a tissue section.

Embodiment 6: The method of embodiment 1, wherein the fixing of whole cells takes place at room temperature for 5-20 minutes.

Embodiment 7: The method of embodiment 1, wherein the permeabilizing of whole cells takes place on ice for 1-10 minutes.

Embodiment 8: The method of embodiment 1, further comprising sequencing at least a portion of the mitochondrial genomes of the single cells to identify one or more mutations relative to a reference mitochondrial genome.

Embodiment 9: The method of embodiment 1, further comprising measuring expression of one or more genes in the single cells.

Embodiment 10: The method of embodiment 1, further comprising detecting chromatin accessibility in the single cells.

Embodiment 11: The method of embodiment 1 further comprising:
a) sequencing at least a portion of the mitochondrial genomes of the single cells to identify one or more mutations relative to a reference mitochondrial genome;
b) measuring expression of one or more genes in the single cells; and
c) detecting chromatin accessibility in the single cells.

Embodiment 12: The method of embodiment 11, whereby cells are assigned to cell lineage groups based on the presence of the mtDNA mutations.

Embodiment 13: A method of determining a cell lineage and determining the cell state of single cells in a multicellular eukaryotic organism comprising:
a) detecting mutations in mitochondria genome sequencing reads of single cells obtained from a subject;
b) measuring gene expression of those single cells; and
c) detecting chromatin accessibility in those single cells;
whereby cells are assigned to lineage and/or status groups based on presence of the mutations and the cell state in the single cells.

Embodiment 14: The method of embodiment 13, wherein the method comprises processing whole cells with fixation and mild permeabilization.

Embodiment 15: The method of embodiments 13 or 14, wherein the method comprises fixing the cells in a buffer comprising 0.01-1% formaldehyde in DPBS, with 0.001-2% bovine serum albumin (BSA) and 0.1-1 U/ul RNase Inhibitor (RI).

Embodiment 16: The method of embodiment 15, wherein the fixing of whole cells takes place at room temperature for 5-20 minutes.

Embodiment 17: The method of embodiments 15 or 16 wherein the method comprises fixing the cells in a buffer comprising 0.1% formaldehyde in DPBS, with 0.04% bovine serum albumin (BSA) and 0.2 U/ul RNase Inhibitor (RI); and the reaction incubates at room temperature for 10 minutes.

Embodiment 18: The method of embodiments 15 or 16 wherein the method comprises fixing the cells in a buffer comprising 0.1% formaldehyde in DPBS, with 0.01% bovine serum albumin (BSA) and 0.2 U/ul RNase Inhibitor (RI); and the reaction incubates at room temperature for 10 minutes.

Embodiment 19: The method of any one of embodiments 13-18, wherein the method comprises permeabilizing the fixed cells in a buffer comprising 10 mM Tris, 10 mM NaCl, 2 mM MgCl₂, 0.1% NP40, 1 mM DTT, and 1 U/ul RI.

Embodiment 20: The method of any one of embodiments 13-18, wherein the method comprises permeabilizing the fixed cells in a buffer comprising 10 mM Tris, 10 mM NaCl, 2 mM MgCl₂, 0.1% NP40, 1 mM DTT, 0.01% BSA, and 1 U/ul RI.

Embodiment 21: The method of embodiment 19 or 20, wherein the permeabilizing the fixed cells takes place at on ice for 5 minutes.

Embodiment 22: The method of any one of embodiments 13-21, wherein the method comprises Tn5 transposase-mediated tagmentation of nucleic acid in the fixed and permeabilized cells.

Embodiment 23: The method of embodiment 22, wherein tagmentation is performed in an ATAC buffer B comprising 1% glycol-diosgenin (ATACH-GDN) and Tn5 transposase.

Embodiment 24: The method of embodiment 22, wherein tagmentation is performed in an ATAC buffer B comprising 0.1-2% glycol-diosgenin (ATACH-GDN) and Tn5 transposase.

Embodiment 25: The method of embodiment 22, wherein the tagmentation is performed at 35-50° C. for 0.5-2 hours.

Embodiment 26: The method of claim any one of embodiments 1-25, wherein retention and/or availability of mitochondrial DNA (mtDNA) is at least 2× greater than when the fixation, permeabilization, and tagmentation are performed in the reference buffers of Table 2.

Embodiment 27: The method of embodiment 22, wherein the Tn5 transposase comprises a barcoded oligonucleotide tag that becomes ligated to a nucleic acid fragment generated by Tn5 transposase nucleic acid cleavage.

Embodiment 28: The method of any of embodiments 13-27, wherein the method comprises processing Tn5 tagmented cells for Gel bead-in-emulsion (GEM) generation and barcoding, wherein an assay for transposase accessible chromatin (ATAC) library is generated and used for both direct sequencing and mtDNA hybridization.

Embodiment 29: The method of any of embodiments 13-28, wherein detecting chromatin accessibility in the single cells comprises single cell ATAC-seq (scATAC-seq).

Embodiment 30: The method of any of embodiments 13-29, wherein measuring gene expression of the single cells comprises single cell RNA sequencing (scRNA-seq).

Embodiment 31: The method of embodiment 13, wherein the method comprises contacting the ATAC library product with four sets of DNA probes, and purification and amplification of the hybridized product.

Embodiment 32: The method of embodiment 31, wherein the sets of DNA probes each cover the whole mitochondrial genome.

Embodiment 33: The method of embodiment 31 or 32, wherein each set of DNA probes contains biotinylated oligos that are 110-160 bp long and the sets of probes are staggered by 10-50 bp with each other over the length of the mitochondrial genome.

Embodiment 34: The method of any of embodiment 31-33, wherein each set of DNA probes contains biotinylated oligos that are 120 bp long and the sets of probes are staggered by 30 bp with each other over the length of the mitochondrial genome.

Embodiment 35: The method of embodiment 13, wherein the calling of a mitochondrial genome mutation comprises:
a) detecting a mutation in a sequence that uniquely maps to the mitochondrial genome; wherein
b) the mutation base is detected in reads of both strands;
c) the mutation base is in a read that is greater than 30 nucleotides;
d) the eUMI for each sequenced fragment is extracted and the percentage of mutant reads in a UMI group (consensus score) is greater than 75; and
e) the UMI group size is 2 or more.

Embodiment 36: The method of embodiment 13, wherein the calling of a mutation comprises:
a) detecting a mutation in a sequence that uniquely maps to the mitochondrial genome; and
b) the mutation base is detected in only one read;
c) the mutation base is in a read that is greater than 30 nucleotides;
d) the eUMI for each sequenced fragment is extracted and the percentage of mutant reads in a UMI group (consensus score) is greater than 75;
e) the UMI group size is 3 or more; and
f) there is no strand bias based on a binomial modeling method.

The invention is further described in the following examples, which do not limit the scope of the invention described in the claims.

### EXAMPLES

### Example 1: Deciphering Cell States and Genealogies of Human Hematopoiesis with Single-Cell Multi-Omics

The human blood system is maintained through the differentiation and massive amplification of a limited number of long-lived hematopoietic stem cells (HSCs). Perturbations to this process underlie a diverse set of diseases, but the clonal contributions to human hematopoiesis and how this changes with age remain incompletely understood. While recent insights have emerged from barcoding studies in model systems, simultaneous detection of cell states and phylogenies from natural barcodes in humans has been challenging, which has limited the ability to explore functional differences between HSC clones. Here, an improved single-cell lineage tracing system based on deep detection of naturally-occurring mitochondrial DNA (mtDNA) mutations with simultaneous readout of transcriptional states and chromatin accessibility is introduced. This system is used to define the clonal architecture of HSCs, and map the physiological state and output of these clones. Functional heterogeneity in HSC clones is uncovered, which is stable over months and manifests as differences in total HSC output as well as biases toward the production of different mature blood and immune cell types. The diversity of HSC clones decreases dramatically with age leading to an oligoclonal structure with multiple distinct clonal expansions. This study thus provides the first clonally-resolved and cell-state aware atlas of human hematopoiesis at single-cell resolution revealing an unappreciated functional diversity of human HSC clones both in young and aged individuals and more broadly paves the way for refined studies of clonal dynamics across a range of tissues in human health and disease. The following describes aspects of the background for the experiments, the experiments themselves and conclusions they support.

Hematopoietic stem cells (HSCs), which sustain life-long production of blood and immune cells, have enormous therapeutic implications for regenerative medicine, and serve as a paradigm for understanding stem cell biology more broadly¹. Recent studies suggest that HSCs are functionally heterogeneous with diverse clonal behaviours, which challenges the classic view of HSCs as having uniform contributions to mature hematopoiesis²⁻⁵. To more deeply understand the functional diversity of HSCs, it is critical to track clonal and subclonal relationships in hematopoiesis to uncover HSC contributions and behaviours in health, as well as in blood diseases, cancers, and in the setting of ageing where HSC functions are frequently perturbed⁶⁻⁸

Transplantation assays have demonstrated clonal heterogeneity in HSCs, but the relevance to homeostatic native hematopoiesis is unclear⁹⁻¹⁰. In model organisms, genetic labelling of HSCs can be used to investigate HSC behaviours in steady state¹¹⁻¹⁶, but even here variation in labelling efficiencies and other experimental parameters have given rise to contrasting views of how HSC clones contribute to the hematopoietic compartments,^{5,16-20}. While genetic labelling of human HSCs is possible in rare settings of transplantation during gene therapy trials, such exogenous labels cannot be routinely used in humans, particularly under native homeostatic conditions²¹.

Somatically-acquired mutations serve as natural barcodes that can be used for retrospective lineage tracing in human samples²²⁻²⁵. Recent studies using whole genome sequencing of colonies composed of differentiated cells derived from single hematopoietic progenitors have advanced the understanding of the clonal dynamics underlying human hematopoiesis^{22,26,27}. However, the original cell state is not preserved with these approaches and such measurements are critical to reveal how cell states impact the behaviour and contributions of HSCs and other cell types to hematopoiesis. Technologies that can simultaneously provide rich cell state readouts in single cells, while also yielding detailed genealogical information from natural cellular barcodes would, in principle, overcome this limitation. It has been previously demonstrated that there is utility and potential for mitochondrial DNA (mtDNA) mutations to serve as natural cellular barcodes in humans²⁸⁻³². However, existing methods can only detect a limited subset of mtDNA mutations, hampering the ability to resolve fine-scale subclonal relationships and hierarchies.

Here, a new approach for single-cell Regulatory multi-omics (transcriptomics and chromatin accessibility) with Deep Mitochondrial mutation profiling (~10-fold increase in detection rate), or ReDeeM, is introduced. ReDeeM is applied to generate a clonally-resolved single-cell transcriptomic and accessible chromatin atlas for 135,155 human hematopoietic cells from 4 human donors, which are enriched to ensure appropriate coverage of rare hematopoietic stem and progenitor cell (HSPC) populations. Through this approach, the clonal architecture of human hematopoiesis is defined, while also revealing the contributions of individual HSC clones to overall and lineage-specific output. Finally, how these patterns vary with human ageing is assessed.

Single-Cell Deep mtDNA Mutation Discovery with Concomitant Multi-Omics

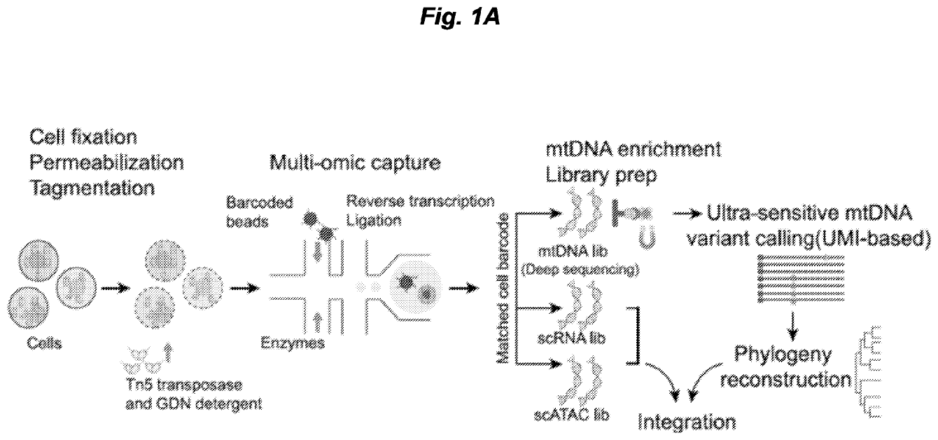

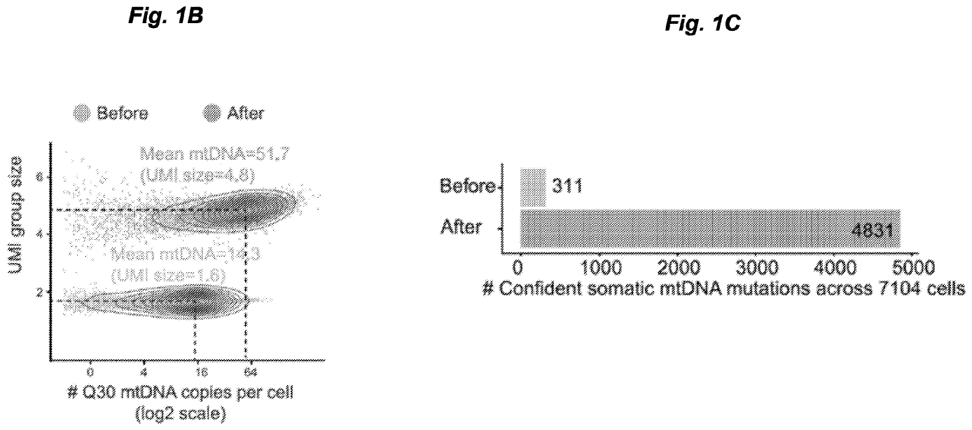

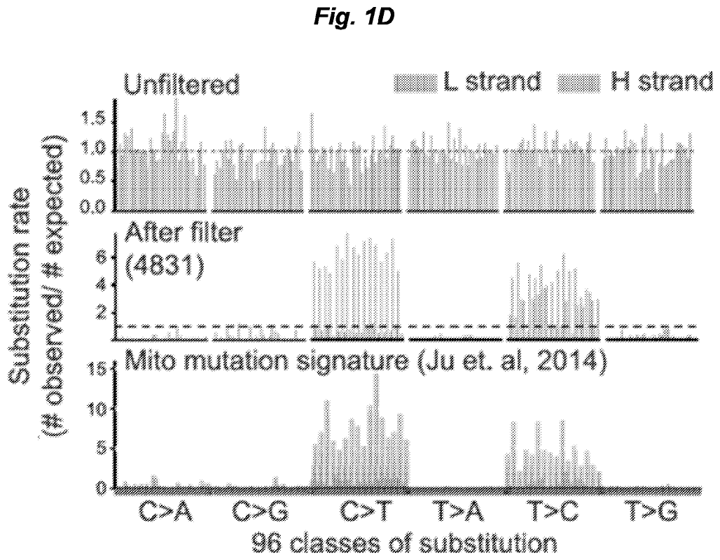

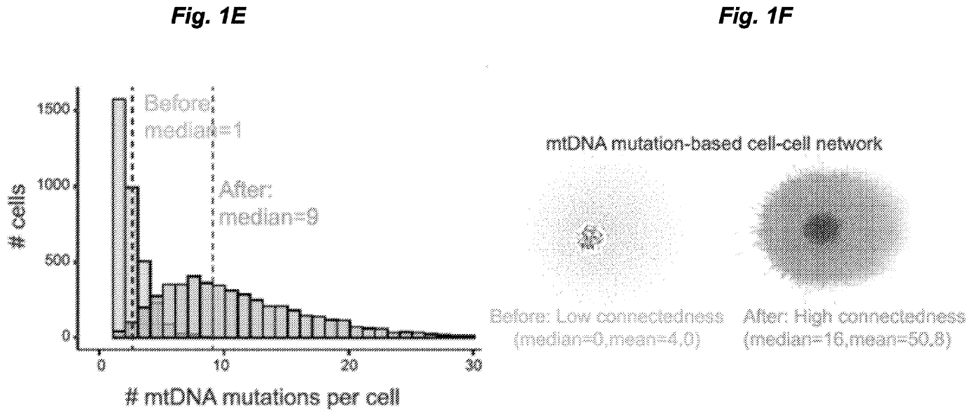

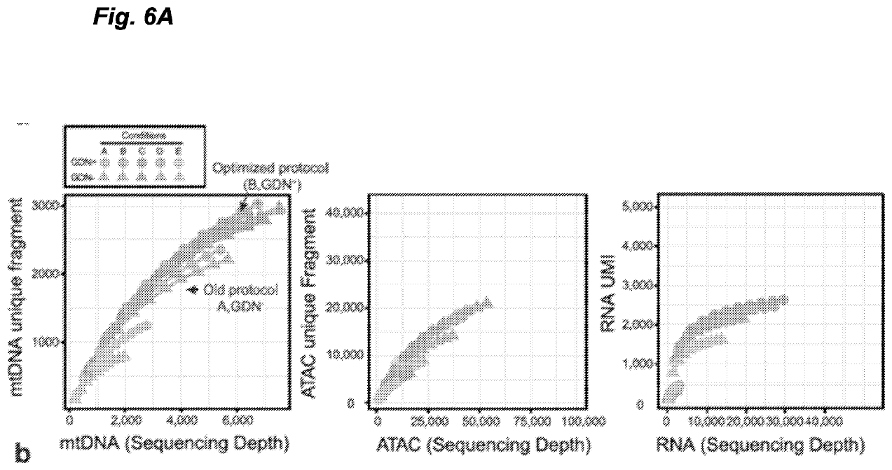

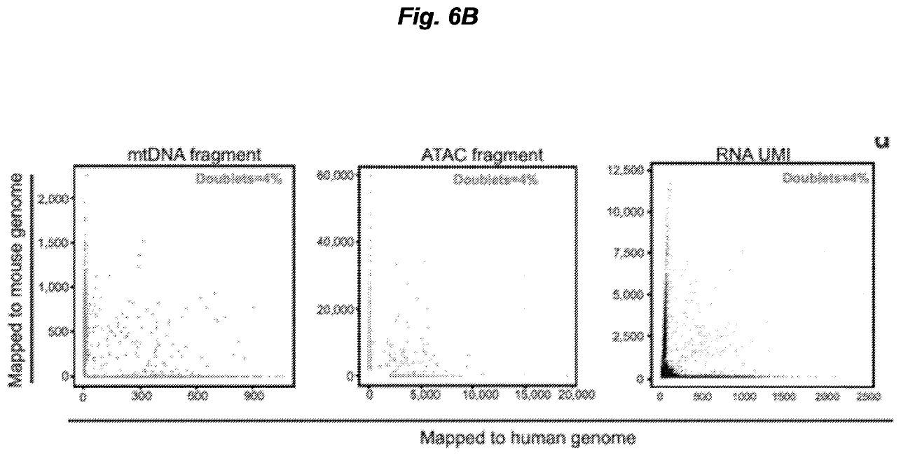

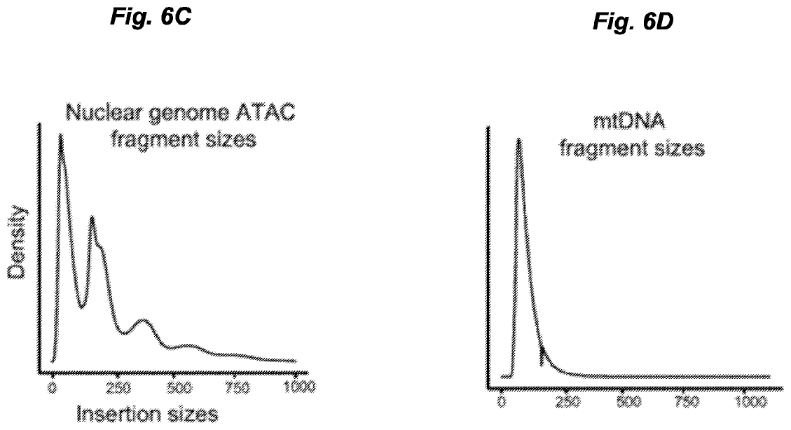

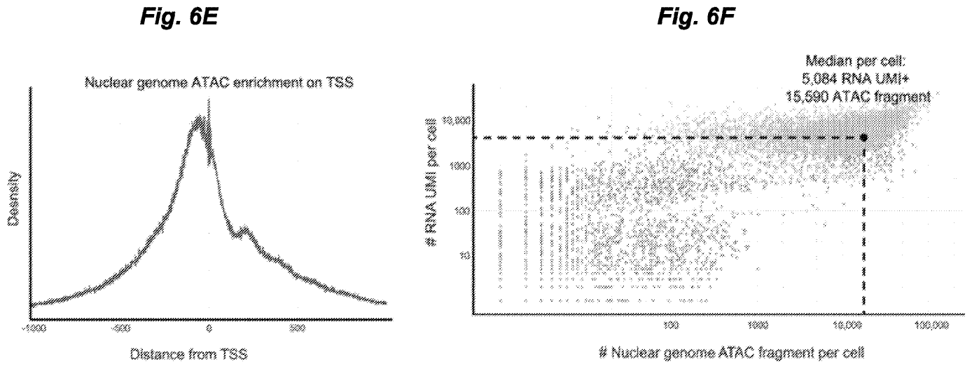

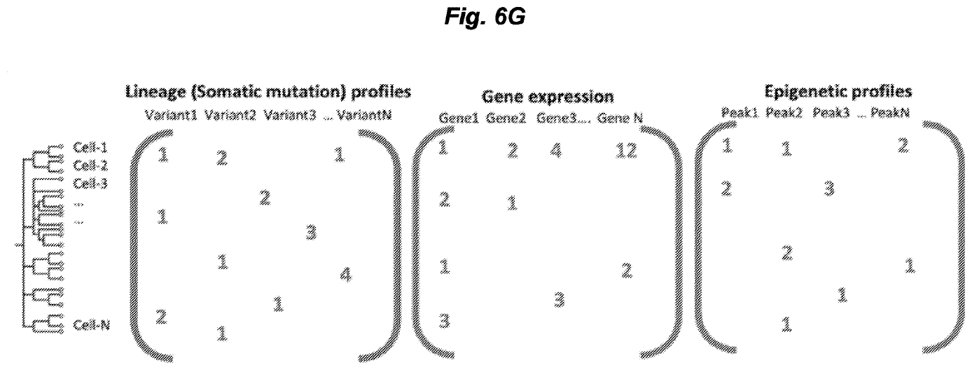

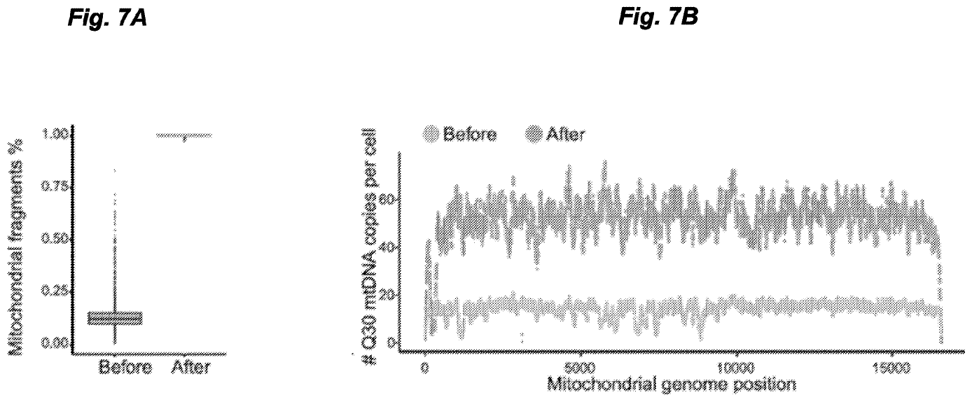

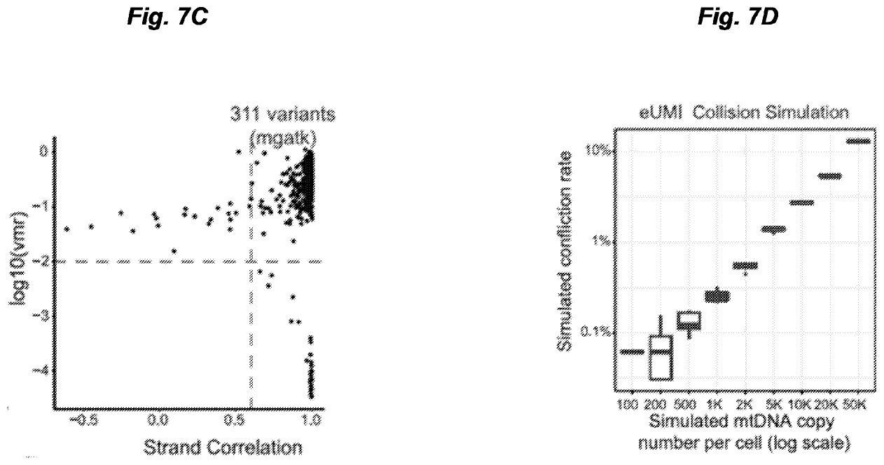

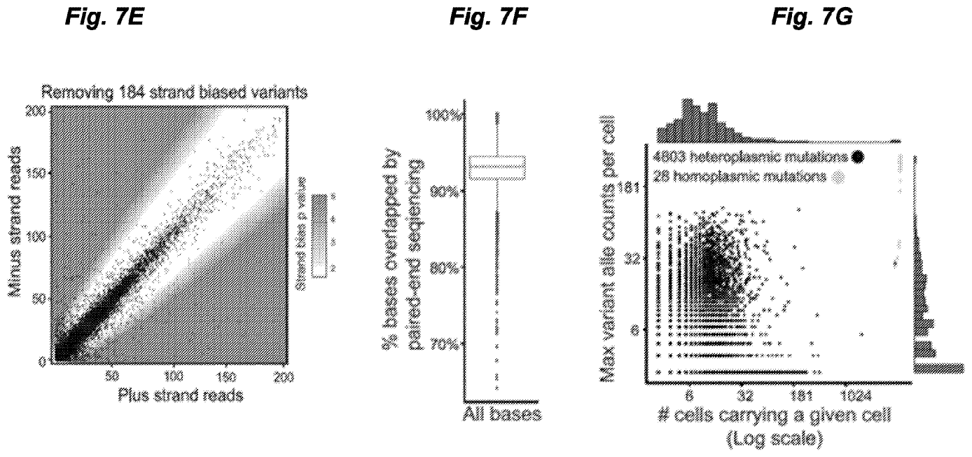

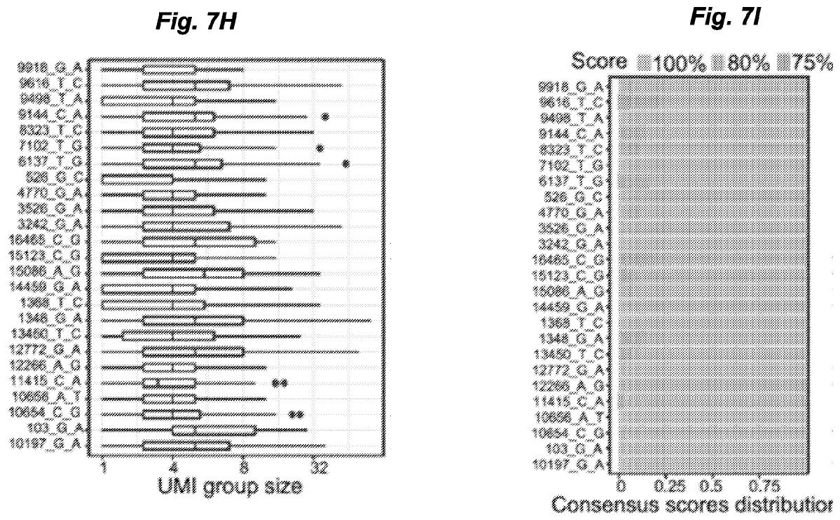

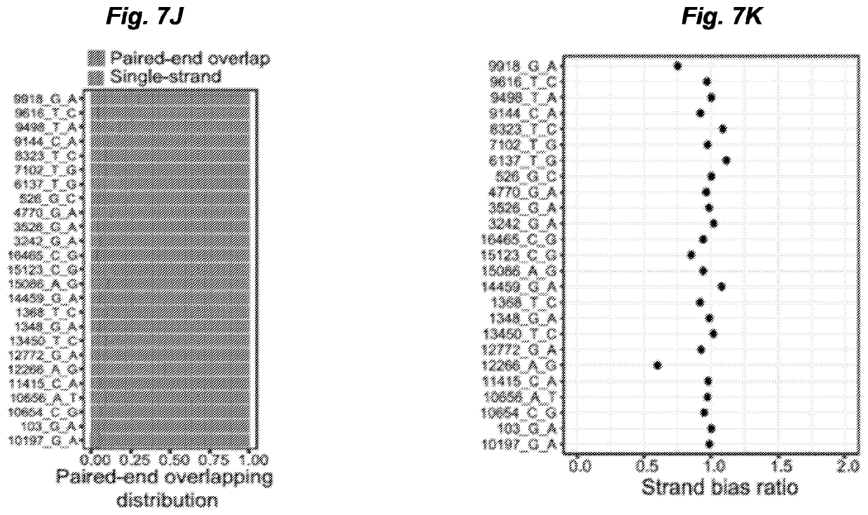

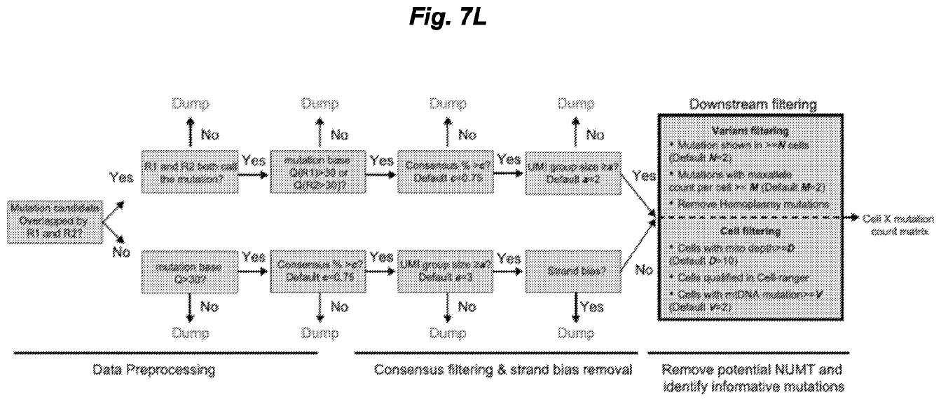

A number of features make mtDNA uniquely well-suited as a natural evolving barcode, including the compact nature of its genome (~16.7 kb), high copy number (100s-1000s of copies per cell), and high rate of spontaneous mutation (estimated to be 10- to 100-fold greater than nuclear DNA)^{28,33,34}. Accordingly, there have been a number of efforts to utilise mtDNA mutations as natural cellular barcodes for lineage tracing and clonal inference that have provided insights into processes such as studies of blood cancers^{29,31,35}. However, the resolution of the resulting phylogenetic analyses has faced substantial technical limitations. In particular, the ability to detect rare mtDNA mutations found in specific subclones is limited due to considerable challenges in library preparation methods, which lead to low coverage depth of mtDNA, and an inability to discriminate sequencing artefacts from true variants. To improve the ability to call a fuller set of mtDNA mutations, single molecule consensus correction is used, which can minimise the impact of sequencing and PCR errors³⁶⁻⁴⁰. The key features of this system are the following: (1) an optimised protocol to maximise yield of mtDNA, (2) a specific enrichment of the mtDNA library that can be subject to very high sequencing coverage, (3) unique molecular identifiers that label individual mtDNA molecules allowing for the use of error corrections to enable high-precision calling of mtDNA mutations, (4) a robust inference algorithm that uses the deeper and improved mtDNA mutation detection for phylogenetic reconstructions, and (5) concomitant scRNA-seq and scATAC-seq that link phylogenetic relationships and cell-state readouts. To achieve these technical goals above, ReDeeM was developed. The droplet-based single-cell multi-ome of the 10× Genomics platform was modified by using whole cells and further optimised protocols that maximise mtDNA coverage, while preserving the scRNA-seq and scATAC-seq library quality (FIG. 1a, FIG. 6, Methods). mtDNA specific probes were designed tiling the mitochondrial genome for hybridization-based capture (Methods). Three separate libraries (mtDNA, ATAC, RNA) are generated for sequencing with matched cell barcodes for downstream integration (FIG. 1a). The cell barcode plus starting and ending positions of the mtDNA fragments serve as endogenous unique molecular identifiers (eUMI) without the need for artificial barcodes (eUMI collision rate <0.5%, Methods, FIG. 7D). The eUMI plays a critical role in grouping sequencing reads and implementing single-molecule consensus variant calling strategies. Based on eUMI, an open-source computational pipeline (redeemV and redeemR package) was developed for consensus mtDNA mutation calling with single-cell multi-omic profiling (FIG. 7I).

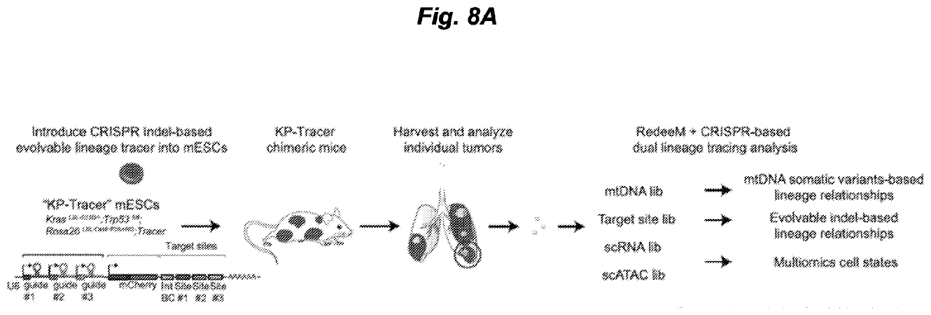

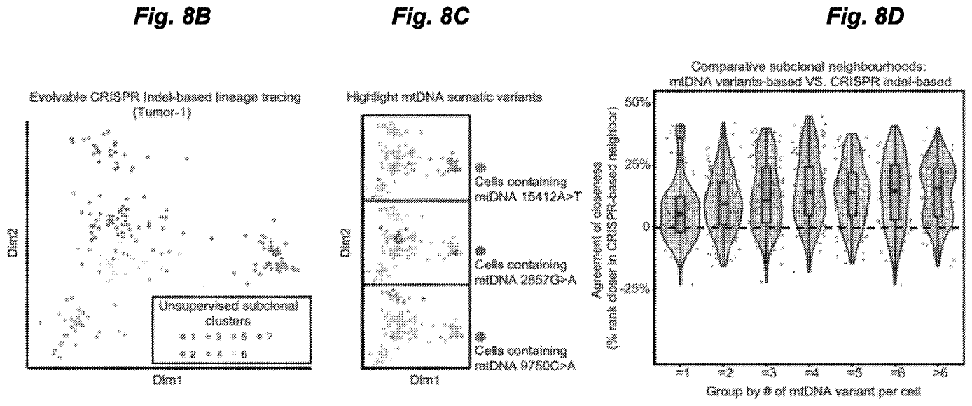

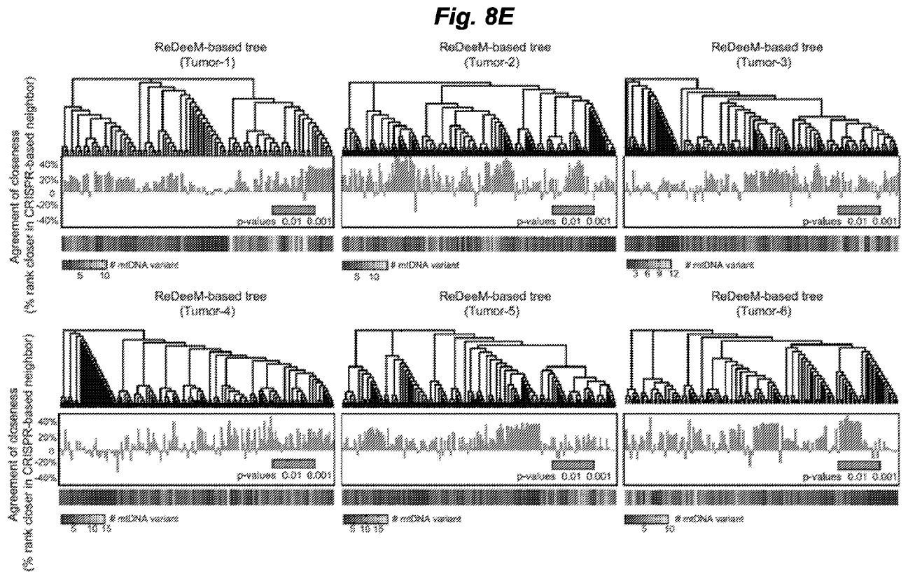

As an initial benchmark of ReDeeM, 7,104 human CD34⁺ HSPCs were profiled from a healthy young donor (age 31). Deep sequencing of the targeted mtDNA library yielded substantially increased mtDNA fragment coverage (on average 51.7 mitochondrial genome copies per cell vs. 14.3 without enrichment) and an ideal eUMI group size for consensus correction (on average 4.8 raw reads for each eUMI copy vs. 1.6 without enrichment) (FIG. 1b, FIG. 7B). After stringent multi-step filtering, 4,831 high-confidence mtDNA mutations were identified across 7,104 cells, which is more than 10-fold higher than has been achieved with previously reported methods²⁹ (FIG. 1c, FIG. 7c,e,g). These 4,831 mtDNA mutations were further examined and validated as well supported by multiple reads in each eUMI group and had high consensus scores. It was also confirmed that the majority are consistent between overlapped paired-end strands (FIG. 7h-k). Notably, the mutational signatures of these 4,831 mtDNA mutations closely match prior reports of the mtDNA mutational spectra which is strongly enriched for transition point mutations⁴¹ (FIG. 1d). Consequently, each cell presented a far higher number of mtDNA mutations (with a median of 9 vs. 1 without enrichment) that are shared by other cells, which increased the cell-cell connectedness by an order of magnitude (FIG. 1e,f). This improved cell-cell connectedness provides an unprecedented opportunity for fine-scale subclonal and phylogenetic analyses. The data quality of the other two modalities, scRNA-seq and scATAC-seq, were benchmarked from the same cells. Both modalities show excellent capture efficiency with a median of 5,084 transcripts and 15,590 ATAC fragments per cell. The ATAC insertions show expected size distributions, and as expected, are highly enriched at transcription start sites (FIG. 6c,e,f). As an independent test of the accuracy of phylogenetic reconstructions generated by ReDeeM, a dual lineage-tracer experiment was designed with a Kras;Trp53(KP)-drive lung adenocarcinoma lineage-tracer mouse model⁴² where both engineered CRISPR-based evolving barcodes were detected on the nuclear genome as well as naturally occurring mitochondrial somatic mutations by ReDeeM in the same single cells. In the six tumours sampled, the majority (79.9%) of cell-cell connectedness using ReDeeM were significantly supported by indel distance-based neighbourhoods (FIG. 8, Methods). Together, this demonstrates the accuracy of deep mtDNA mutations for faithful phylogenetic reconstruction.

### Cell-State Aware Phylogenetics of Human Hematopoiesis

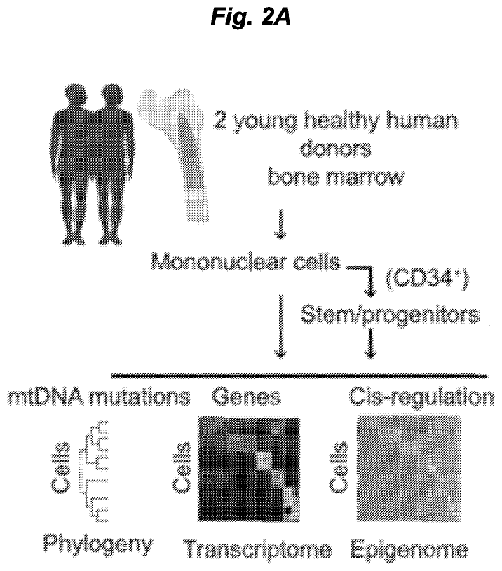

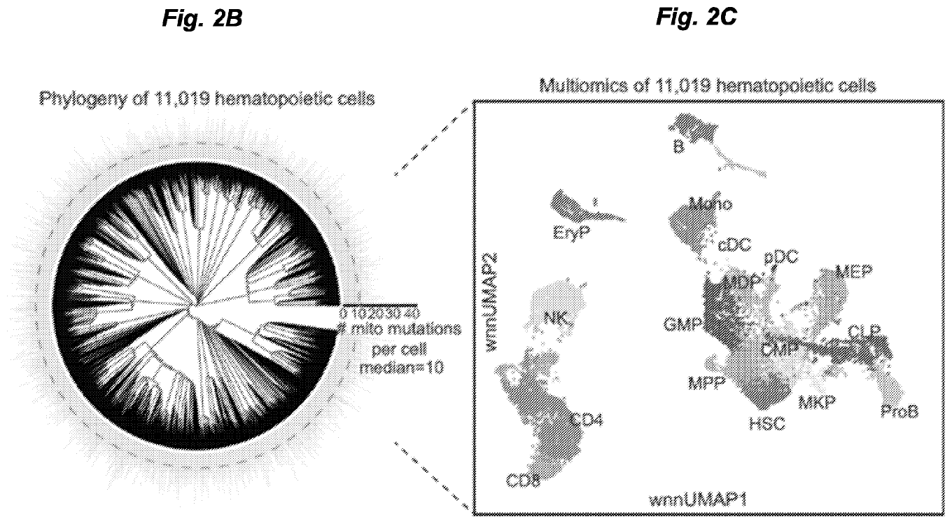

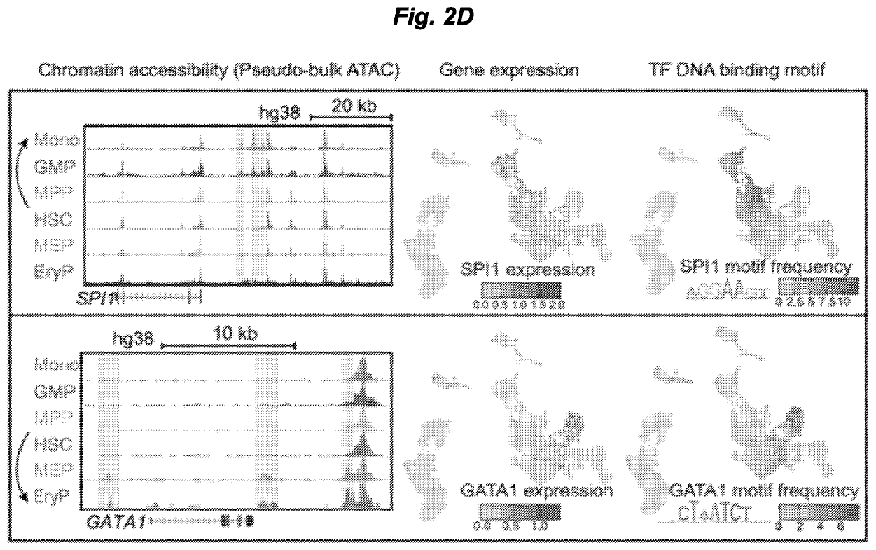

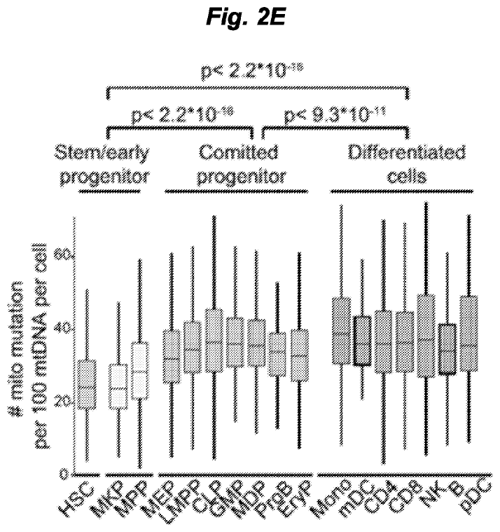

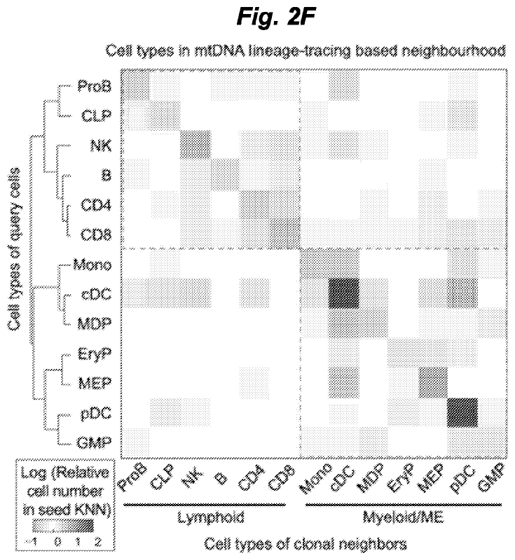

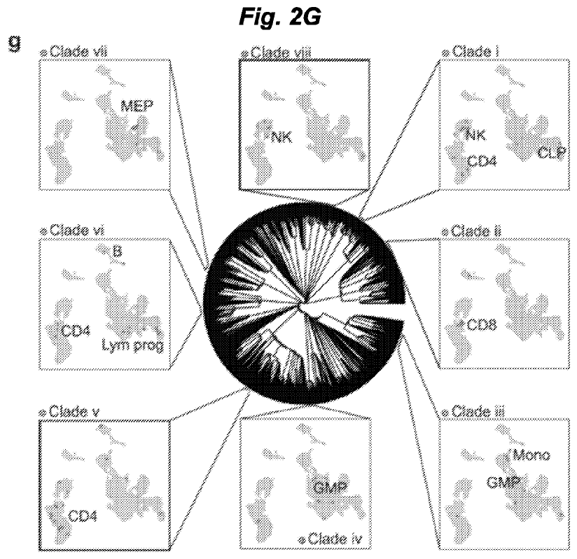

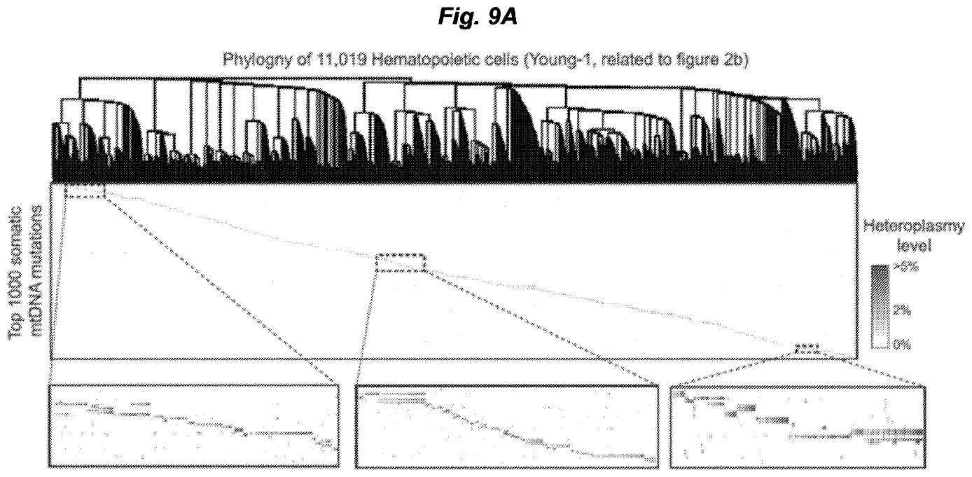

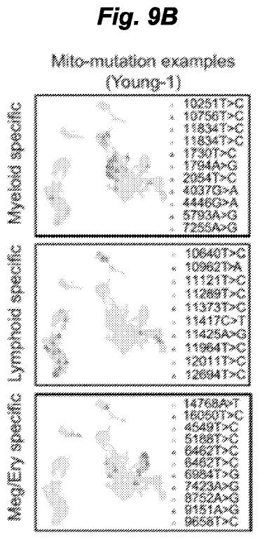

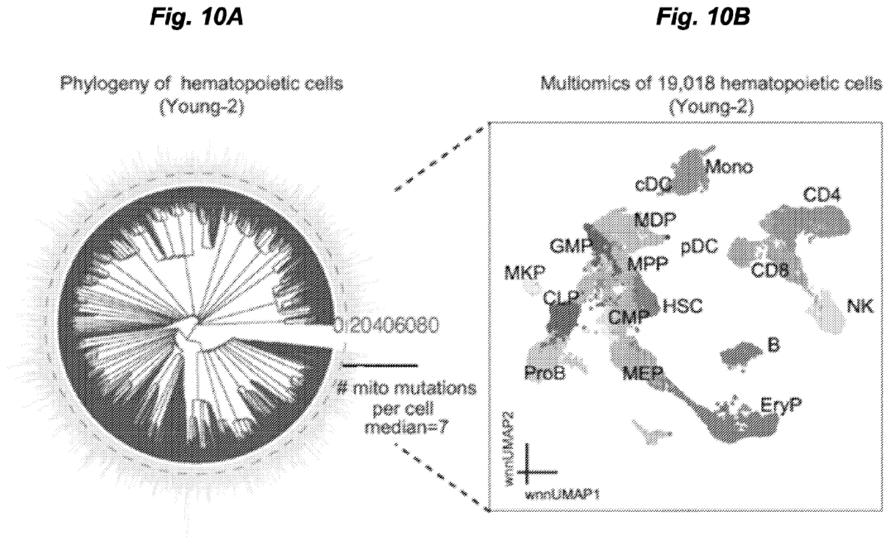

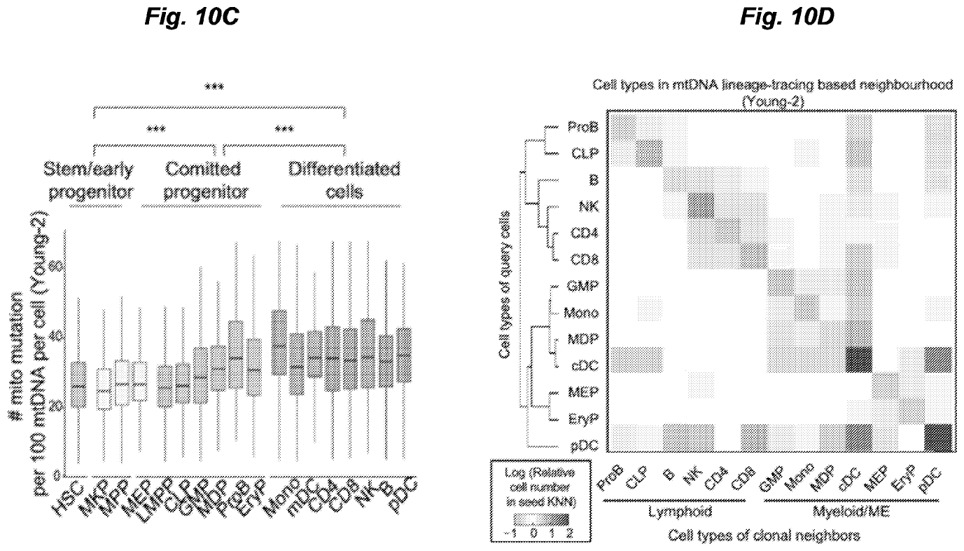

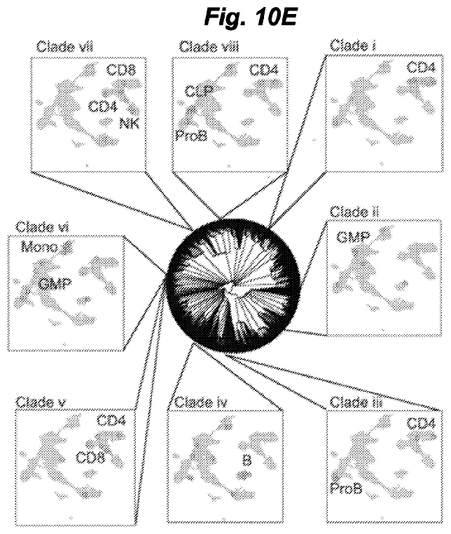

Next ReDeeM was employed to explore human hematopoiesis. Bone marrow aspirates were collected from two healthy young donors, ages 31 (young-1) and 26 (young-2), and isolated mononuclear cells (predominantly differentiated blood cells and precursors) and CD34⁺ HSPCs to ensure robust representation of both undifferentiated and more differentiated cells. 11,009 (5,415 bone marrow mononuclear cells or BMMCs were profiled, along with 5,594 hematopoietic stem and progenitor cells or HSPCs) and 15,101 (7,147 BMMCs, 7,954 HSPCs) hematopoietic cells in young-1 and young-2 respectively for all three modalities, including deep mtDNA mutation profiling (FIG. 2a). 3,896 and 4,803 mtDNA mutations in young-1 BMMCs and HSPCs were confidently identified, as well as 4,087 and 5,137 mtDNA mutations in young-2 BMMCs and HSPCs, respectively. Based on shared deep mtDNA mutation profiles, the phylogenetic trees of each donor's hematopoietic compartment were reconstructed using the neighbour-joining algorithm (FIG. 2b, FIG. 10a, Methods). The resulting trees were highly polyclonal, consistent with recent phylogenetic analysis based on whole nuclear genome sequencing of hematopoietic colonies from healthy donors, and the tree structures were well supported by multiple mtDNA mutations²² (FIG. 9a).

Next, the cell state was assessed using the transcriptomic and epigenomic information available for each leaf (single cell) in the phylogenetic trees. Chromatin accessibility and gene expression in the hematopoietic cells are highly consistent for both donors. Weighted nearest neighbour (WNN) metrics were used to integrate both modalities and identified 17 major hematopoietic cell types/clusters⁴³ (FIG. 2c). Having paired scRNA-seq and scATAC-seq profiles from individual cells also enabled us to explore the regulatory circuits in hematopoietic cell fate decisions. For instance, on the bifurcation paths between the other myeloid lineages and megakaryocyte/erythroid (ME) lineage, how the master transcriptional regulators SPI1 and GATA1 are turned on with specific regulatory elements and subsequently promote differentiation trajectories characterised by increased accessibility of one or the other transcription factor motif was observed (FIG. 2d). Interestingly, the GATA1 motif starts to be activated earlier during HSC differentiation even with low GATA1 expression compared to SPI1, which is consistent with prior studies using RNA-velocity and kinetic analyses^{44,45}. Next, the mtDNA mutation information was incorporated into the cell-state landscape. Interestingly, it was found that HSCs show significantly lower mtDNA mutation burden than more committed progenitors and differentiated cells, arguing that there is acquisition of additional subclonal mtDNA mutations that occur as cells divide during differentiation which, as discussed below, provides an opportunity to explore phylogenetic relationships between different cell types (FIG. 2e, FIG. 10c). Taken together, the data provides the first clonally-resolved and cell-state aware atlas of human hematopoiesis at single-cell resolution, allowing previously unachievable inferences on the regulatory mechanisms underlying this complex differentiation process.

Deep mtDNA Mutation Profiling Reveals Cell-Type Origins

The cell state-aware phylogenetic trees of human hematopoiesis allow us to explore the developmental origins and relationships among different blood and immune cell types, some of which are still incompletely understood or for which there have been controversies^{46,47}. Mapping of the multi-omic data-derived cell type annotations onto the phylogenetic tree revealed that different hematopoietic cell populations were widespread across the tree due to the polyclonal origins. However, interestingly, many fine-scale subclonal structures, or clades were identified (i.e. the full set of cells that descend from a common ancestor and thus encompass branch of a phylogenetic tree), where 1,650 and 2,079 clades are significantly enriched for specific cell types (FDR<0.2, Fold change>2) in the two donors respectively (e.g. CD8⁺ T lymphocyte enriched clades), which are supported by detection of mtDNA mutations that display lineage biases (FIG. 2g, FIG. 9b, 5e). Next, the cell type origins were quantitatively assessed using mtDNA mutation-based nearest neighbour analysis. As expected, the nearest clonal neighbour of most cell types (11 out of 13) is the identical cell type. Notably, this analysis largely reconstructed the hierarchical organisation of the blood cell type origins that have been described and characterised extensively in conventional studies of hematopoiesis⁴⁸ (FIG. 2f). However, some unexpected insights emerged from this analysis. For example, it has been challenging to clearly define the progenitor populations that give rise to conventional and plasmacytoid dendritic cells (cDC and pDC, respectively)^{49,50}. In this data, cDC and pDC show less restricted clonal origins that likely occur between these different types of dendritic cells, as well as their subsets⁵¹. Interestingly, both cDCs and pDCs appear to have a more myeloid-derived origin, which echoes recent lineage tracing studies using reporters and barcoding in mice⁴⁷. Together, this method resolves clonal and subclonal relationships for native steady-state human hematopoiesis, while linking these relationships with rich and robust readouts of cell state.

Unravelling Human HSC Heterogeneity with Multi-Omics

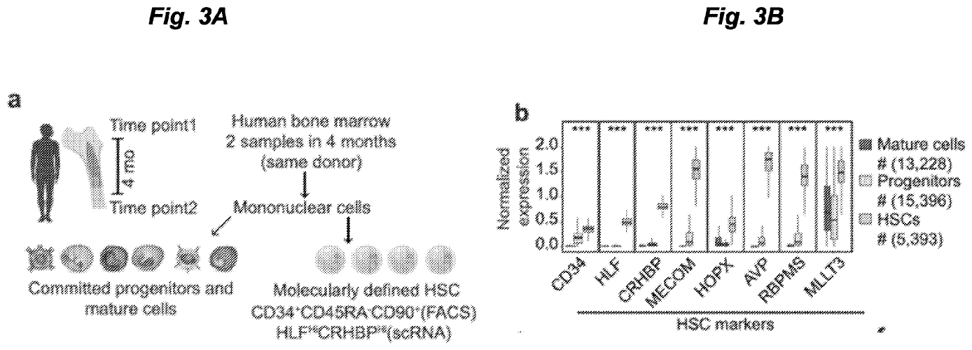

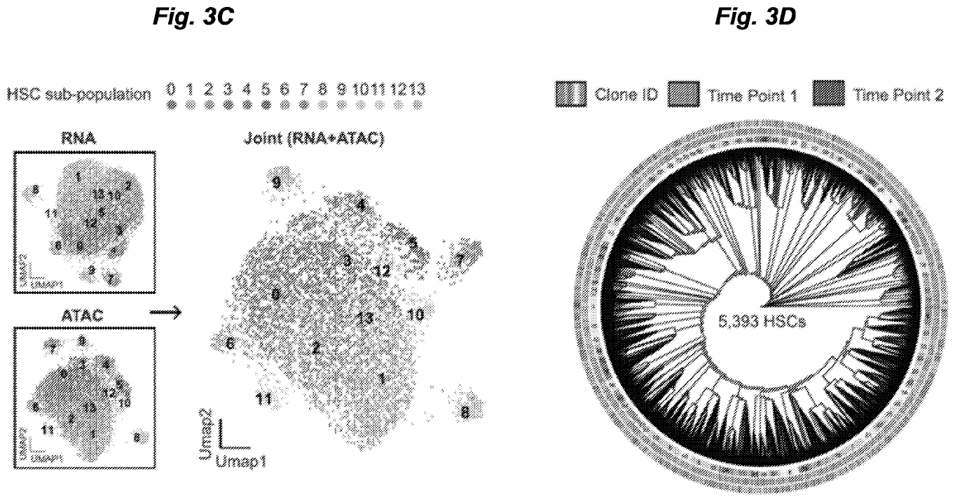

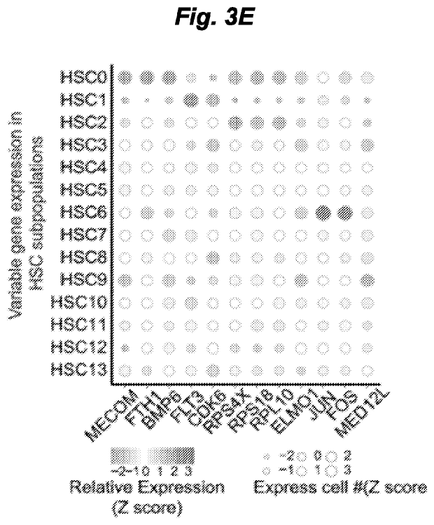

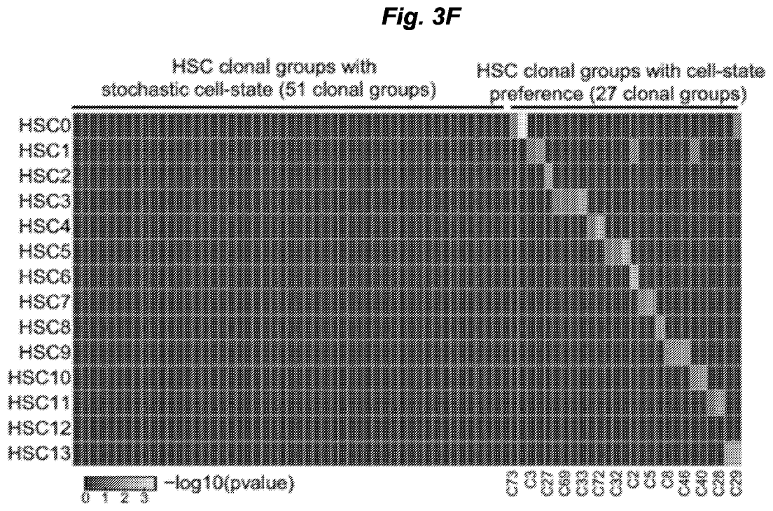

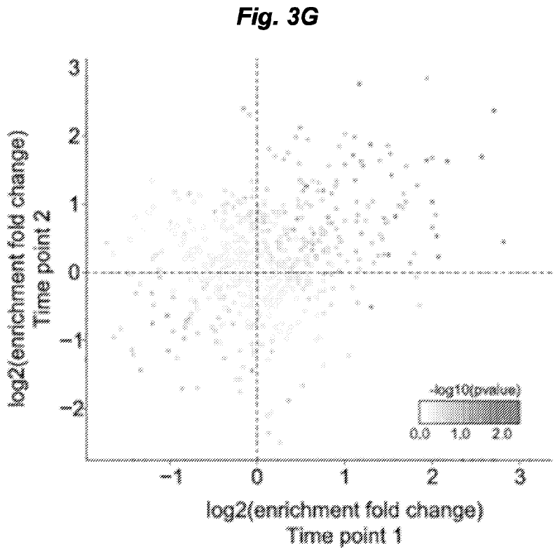

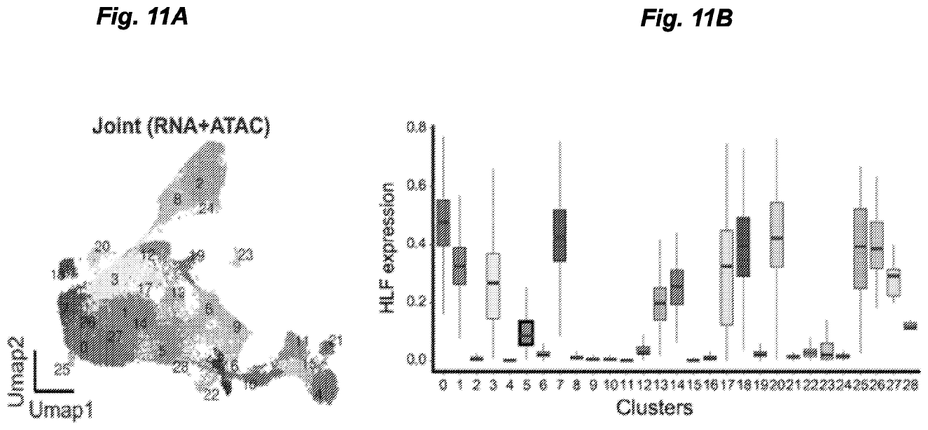

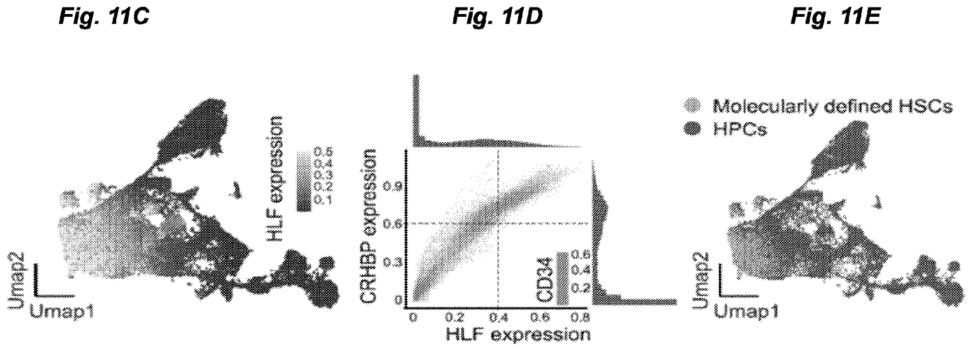

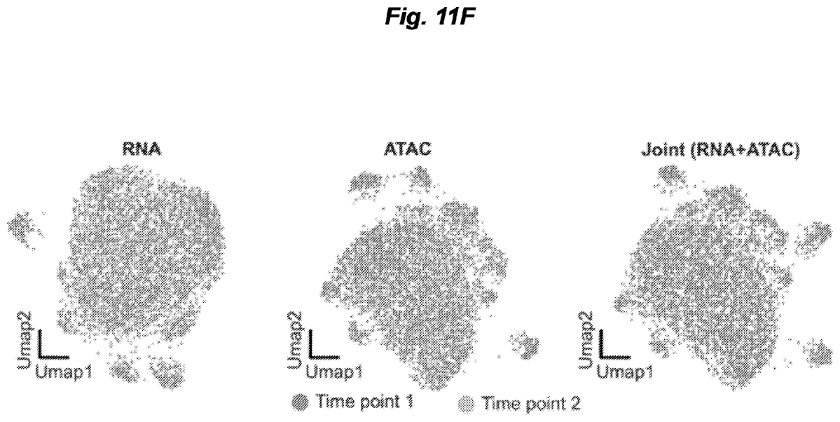

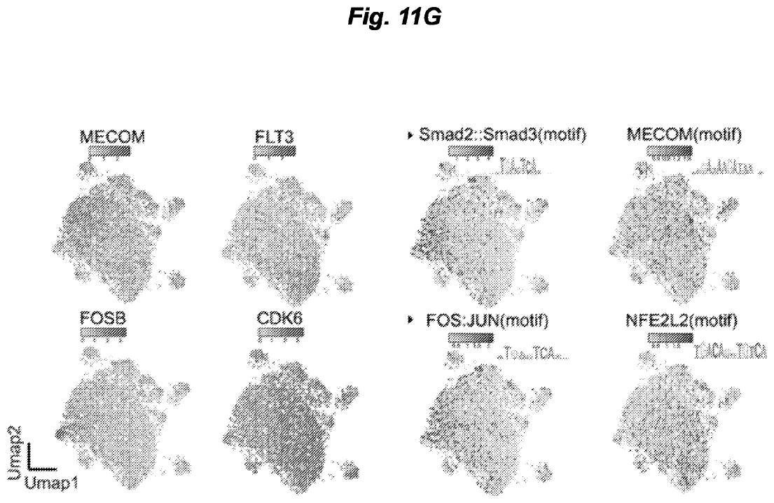

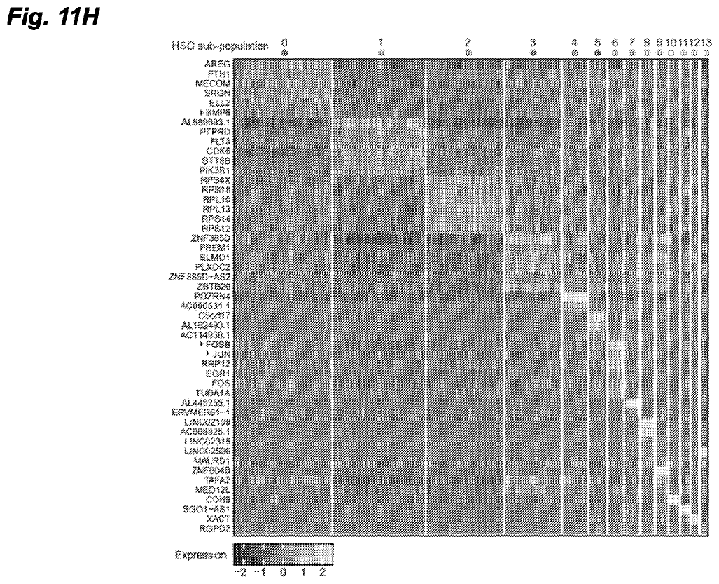

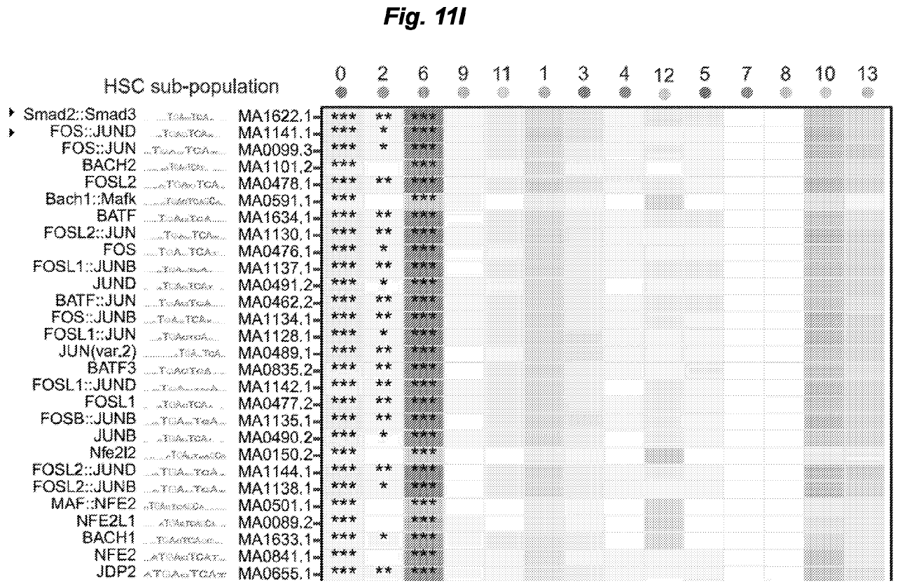

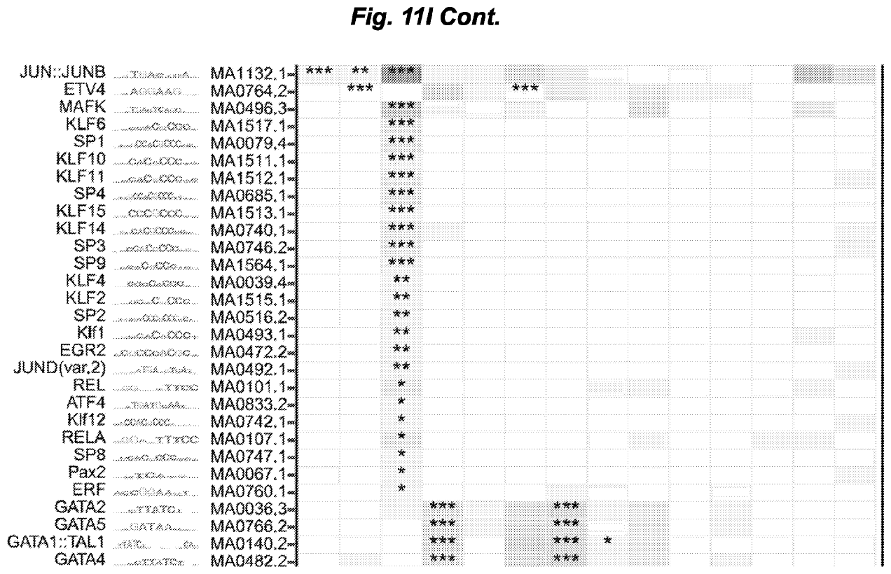

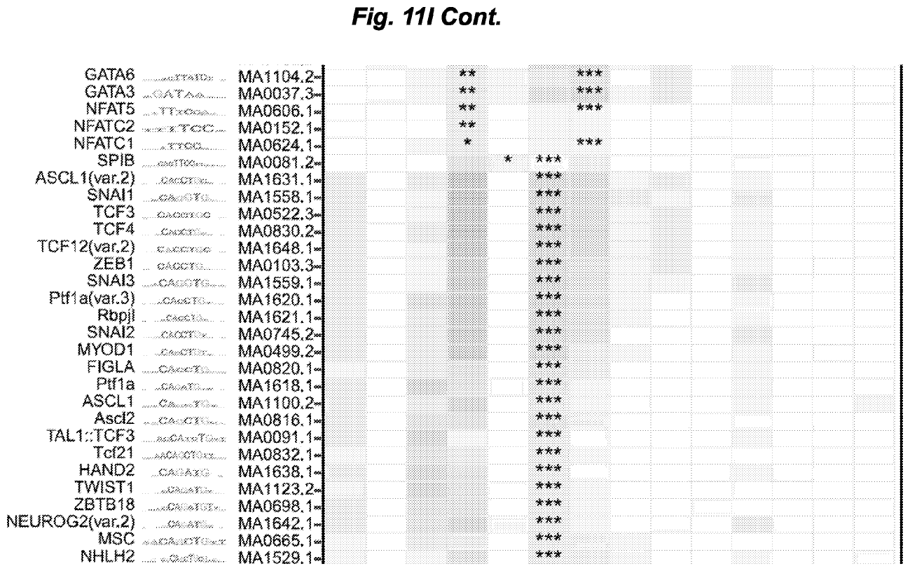

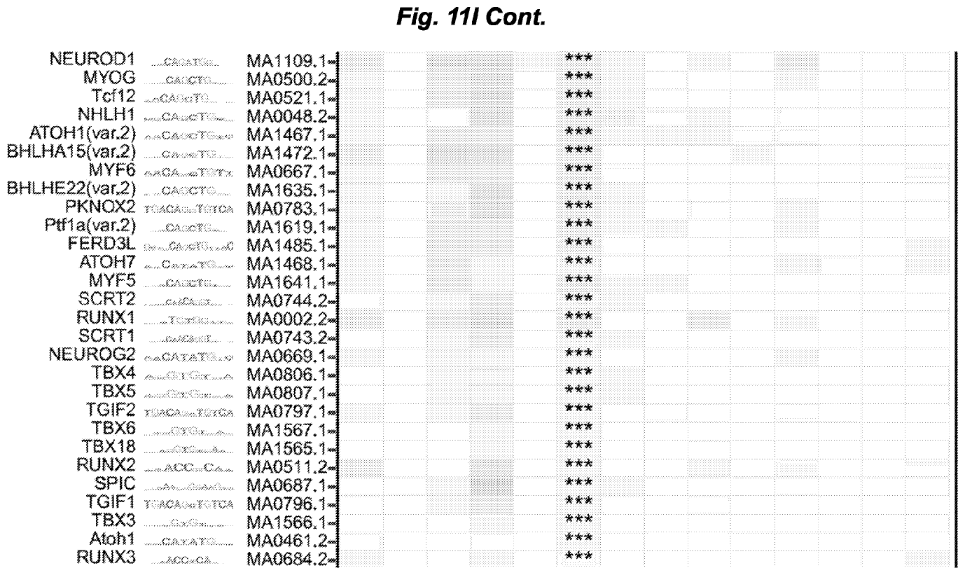

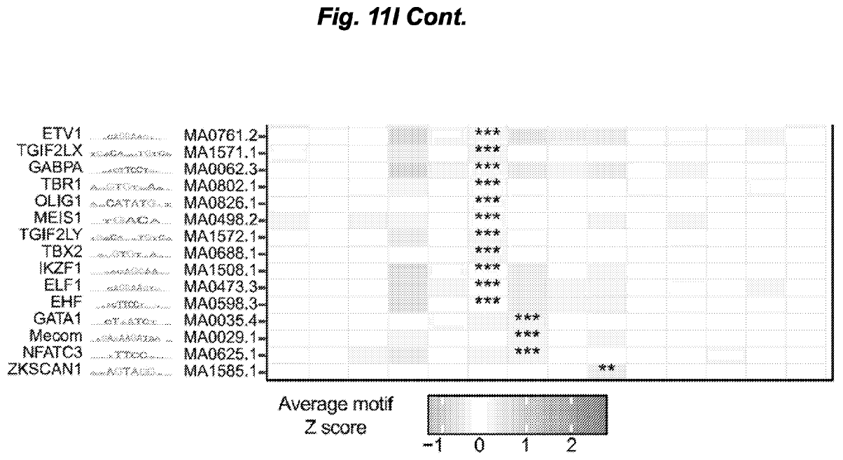

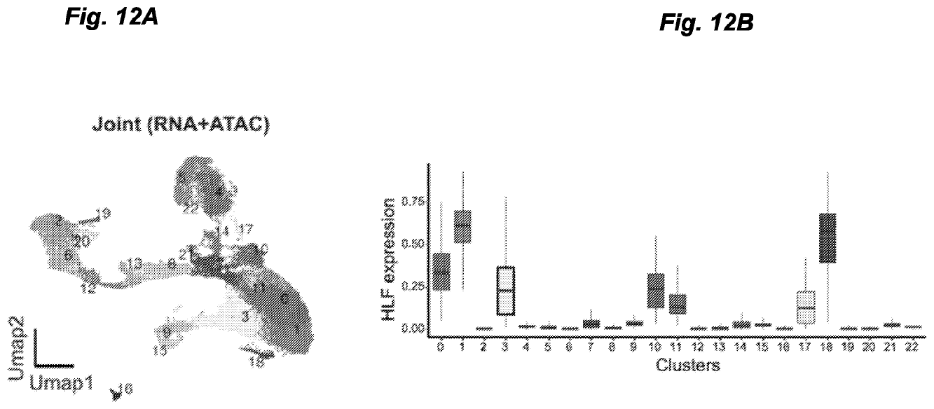

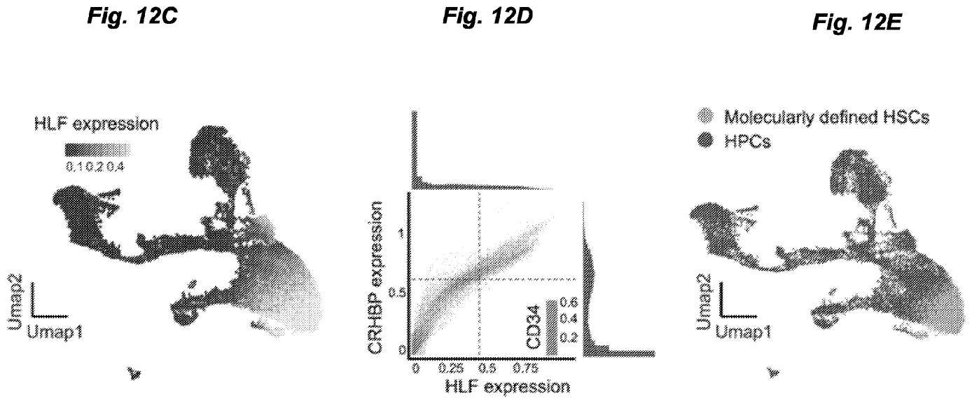

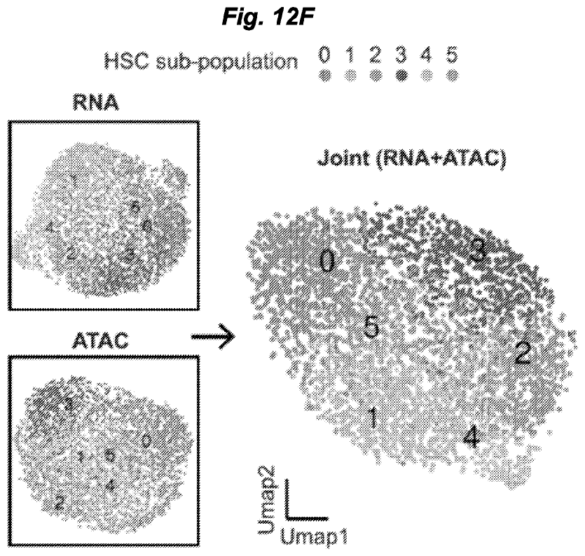

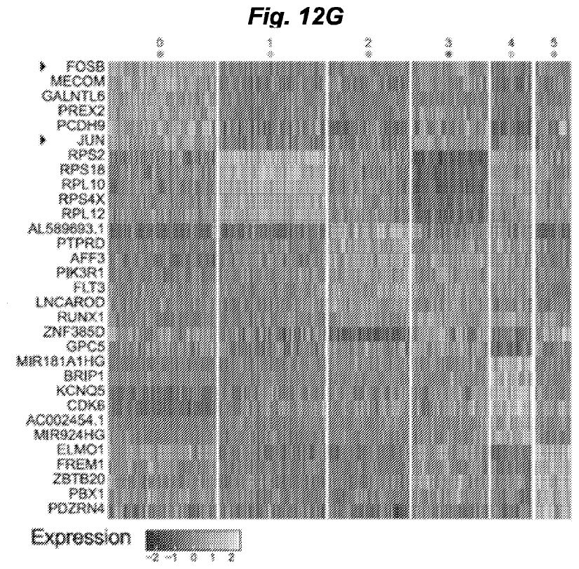

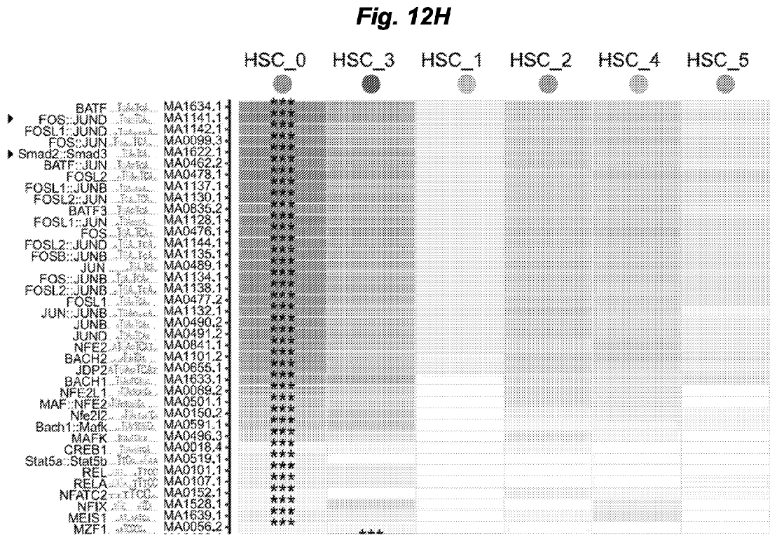

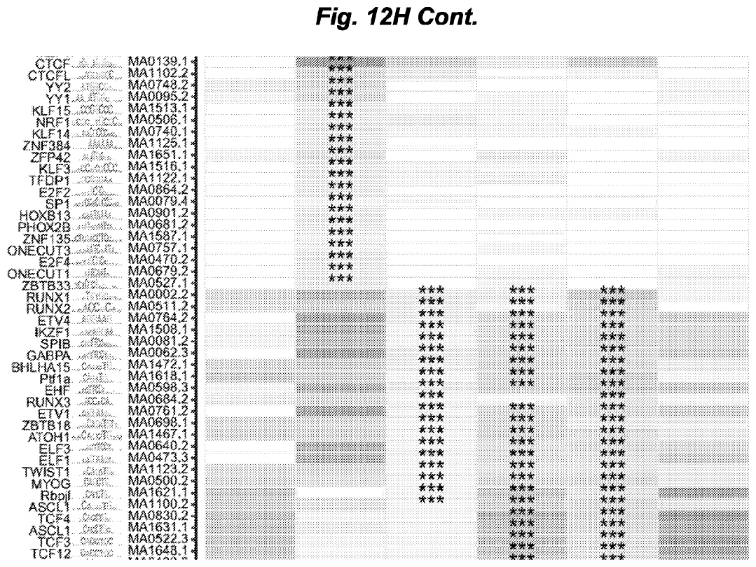

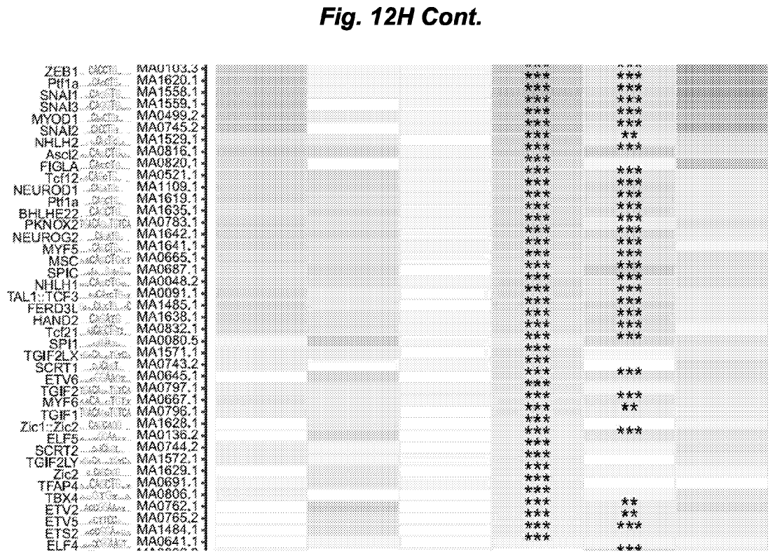

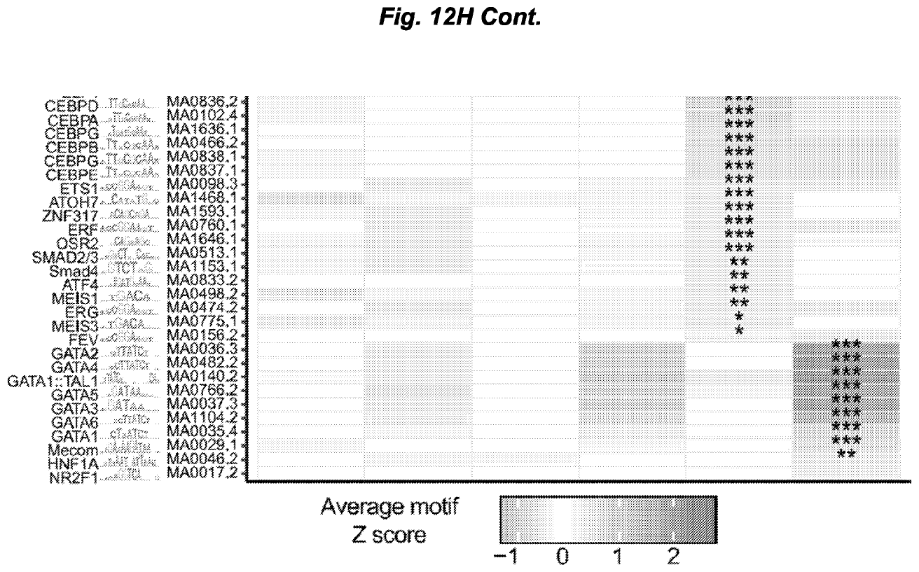

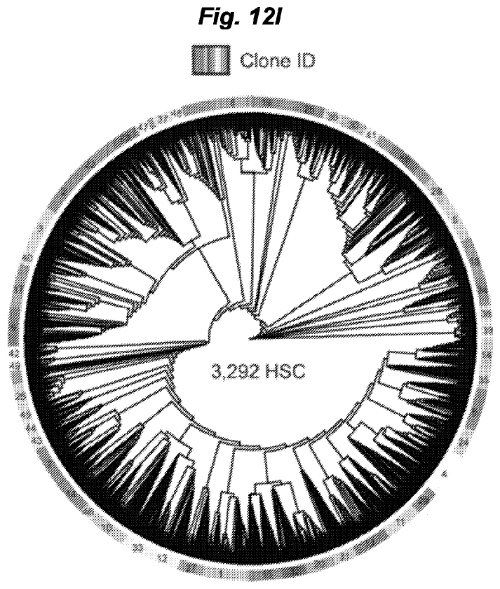

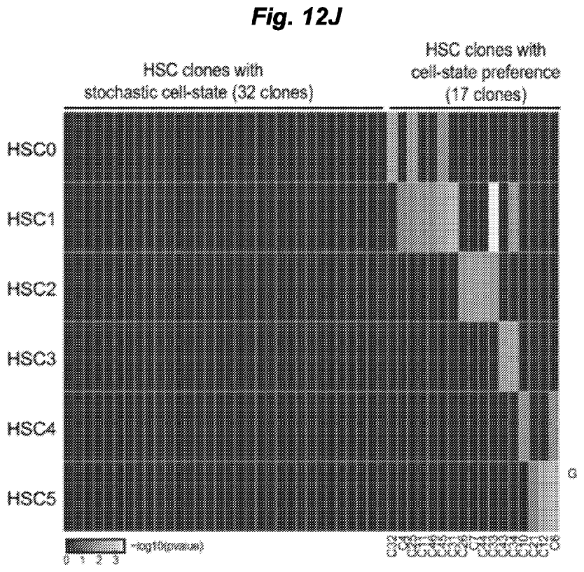

The coupling between more closely related clones in the phylogenetic tree and hematopoietic cell states can arise from two factors. (1) mtDNA mutations that emerge in HSC clones that display lineage bias or (2) mtDNA mutations that are acquired later during differentiation after lineage commitment. The former possibility, or the extent to which HSCs have clonal and functional heterogeneity is of high clinical importance, but remains unclear in native human hematopoiesis. The technical advances made provided a unique opportunity to address these distinct possibilities to specifically dissect HSC heterogeneity. To enhance recovery of HSCs, HSCs were first by FACS sorting and profiling the phenotypic CD34⁺CD45RA⁻CD90⁺ population. These data were filtered for clusters of cells that specifically express HSC marker genes HLF and CRHBP (Methods, FIG. 3a, FIG. 11a-e)^{52,53}. 5,393 and 3,292 HSCs were identified in young-1 and young-2 respectively, which were independently validated by examining expression of other markers known to be specifically enriched in HSCs including MECOM, MLLT3, and RBPMS (FIG. 3b)52,54,55. Importantly, to examine the stability of HSC molecular and behavioural heterogeneity and thus establish a definitive link between the phylogenetic trees and HSC clonal behaviours, the HSCs were sampled twice over the course of 4 months from the same donor (young-1) (FIG. 3a). Unsupervised clustering of HSCs was further performed based on WNN space using combined transcriptomic and accessible chromatin states and identified 14 subpopulations in young-1 (FIG. 3c). Notably, all subpopulations are consistently identified in both ATAC and RNA space, and reproducibly detected in both time points (FIG. 3c, FIG. 11f). Across HSC subpopulations, differentially expressed genes were identified along with differential transcription factor accessibility to uncover regulatory circuits that govern HSC molecular heterogeneity (FIG. 11h-i). For instance, although overall highly expressed in all HSCs, some key HSC genes such as MECOM, FLT3, and CDK6 are differentially expressed across subpopulations (FIG. 3e, FIG. 11g). These genes are known to be important for HSC functions including HSC maintenance, self-renewal, and differentiation, while dysregulation of these factors can contribute to leukemogenesi^{52,56-58}. Several differential pathways were also found across subpopulations with evidence at both the level of gene expression and TF activity changes, such as BMP-SMAD signalling alterations, and changes in AP1 signalling (FIG. 11h-i), reminiscent of prior studies in mice suggesting key roles for these pathways in HSC heterogeneity^{59,60}. Notably, the major HSC subpopulations are reproducible in young-2, but rarer subpopulations were also identified that are specifically found in each individual (FIG. 12a-h). Overall, this data provide a multi-omic resource that allows us to decipher human HSC heterogeneity.

### Resolving Human HSC Clonal Structure and Cell-State Biases

Next, based on the shared mtDNA mutations across 5,393 molecularly-defined HSCs, the phylogenetic tree was reconstructed that revealed the clonal relationships across HSCs. To explicitly study HSC clonal features, the HSC clonal groups were defined by dividing the tree structure into small clades, which are groups of the most closely related HSC clones (FIG. 3d, Methods). The resulting tree shows a balanced polyclonal architecture of HSCs. In total, 78 HSC clonal groups were defined out of 5,393 profiled single HSCs. Notably, the large majority of the HSC clonal groups can be re-observed in the sequential sampling of the same donor, suggesting they provide a sampling that is representative of HSCs that contribute to hematopoiesis over at least several months in vivo, a timescale over which the majority of non-HSC cell types are believed to have turned over at least once.

It is unclear whether different HSCs have heritable cell states or if the variation in HSC states represent stochastic, short lived fluctuations. This data link clonal identity and cell states from the same cells, and thus it was possible to directly measure the distributions of the 78 HSC clonal groups across the 14 multi-omic cell-state-based HSC subpopulations. 51 (or ⅔) of the HSC clonal groups are stochastically distributed across different HSC states, whereas 27 (or ⅓) of the HSC clonal groups reveal significant enrichment in one or a small number of specific state-subpopulations (FIG. 3f). Interestingly, the HSC clone-to-subpopulation enrichment is significantly correlated in the same donor across the two time points which span 4 months (FIG. 3g), suggesting that HSC biases can be sustained over at least a period of months in humans. The clonal structure for HSCs in young-2 was further examined. Consistent with the analysis of young-1, a polyclonal structure with both stochastic HSC variation and other clones demonstrating cell-state preference with similar proportions to what was observed in young-1 (FIG. 12i,j) was also observed. Together, the HSC clones were surveyed with their molecular states and suggested a partially heritable state-preference for approximately one third of HSC clones that were analysed, which is stable over the course of months.

### Identifying Differentiated Progeny of HSCs to Assess Clonal Output and Lineage Biases

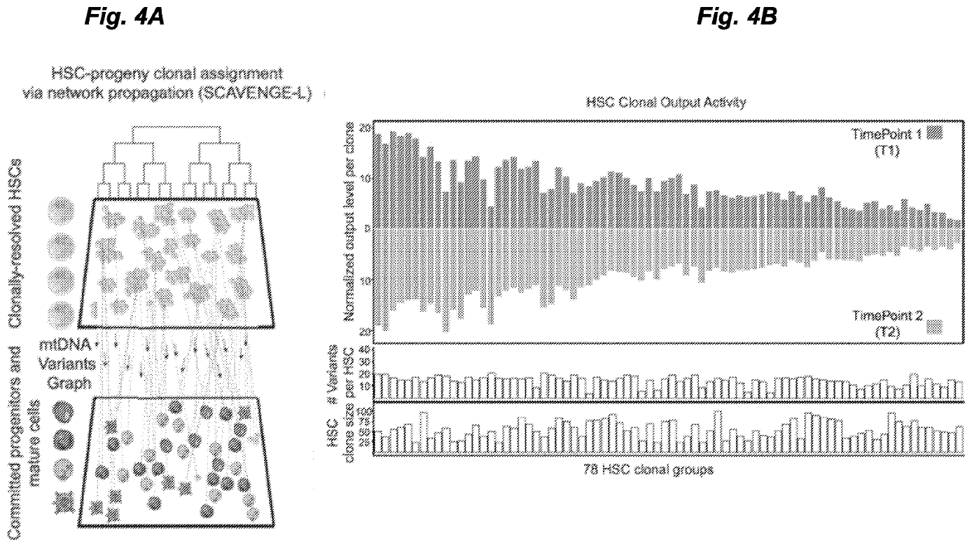

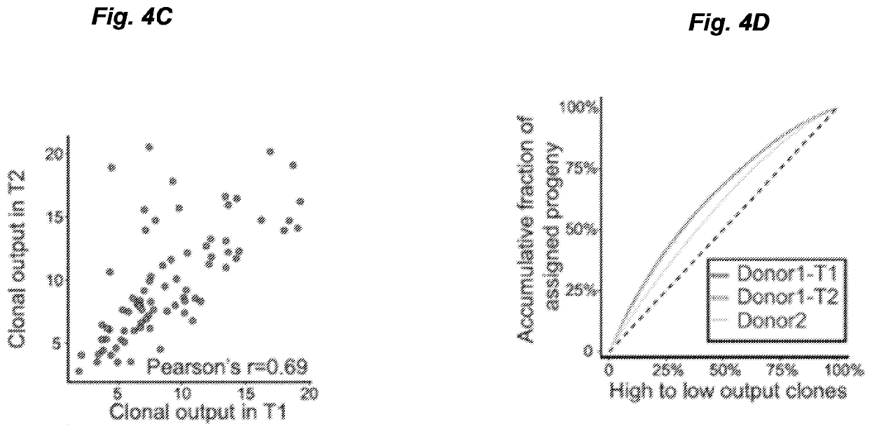

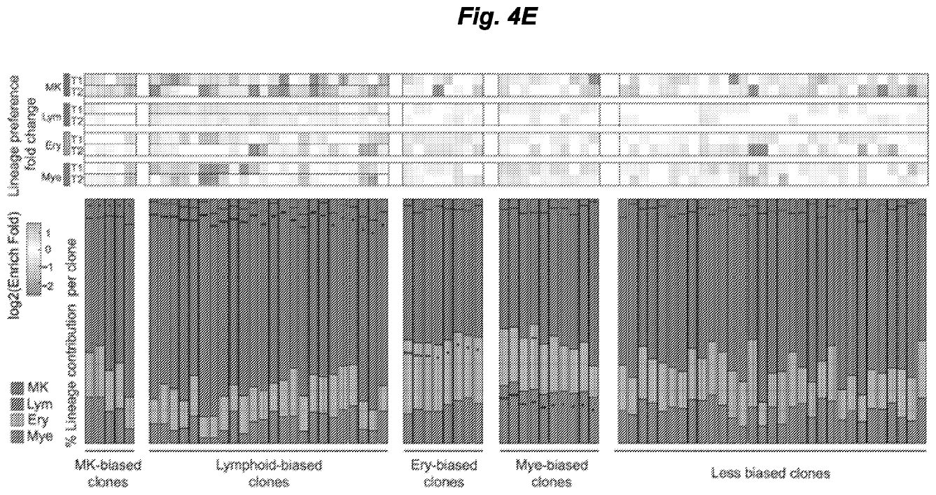

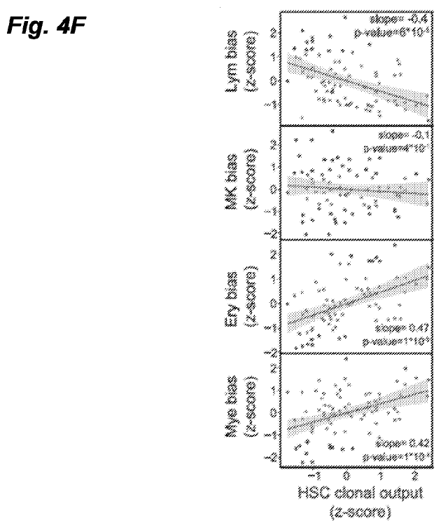

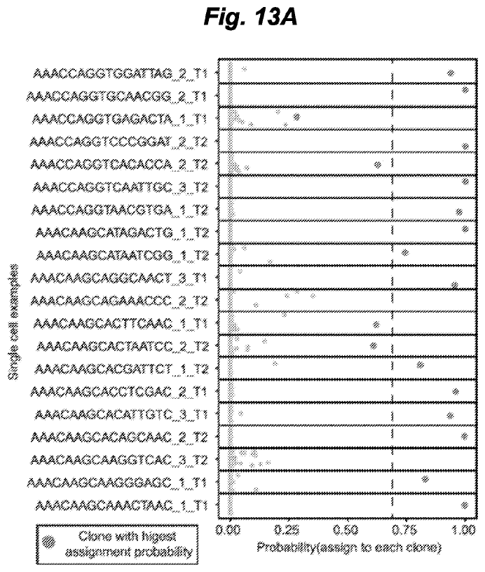

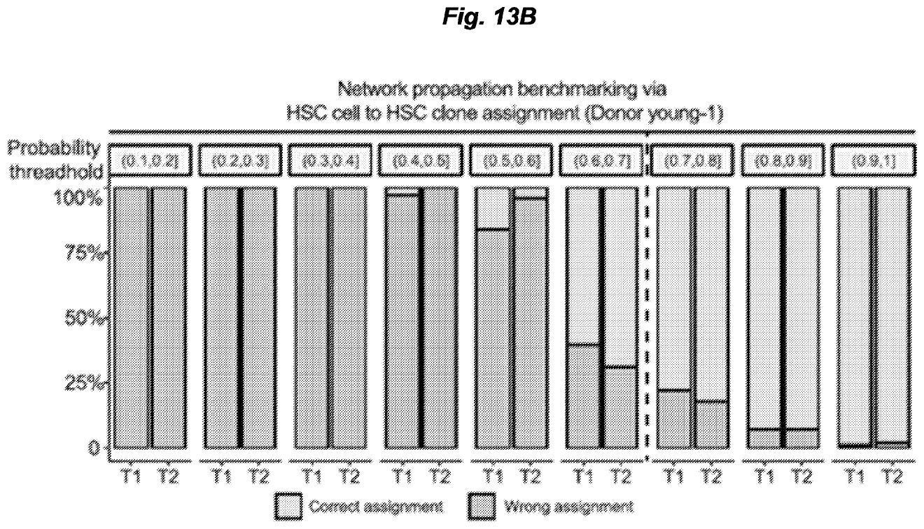

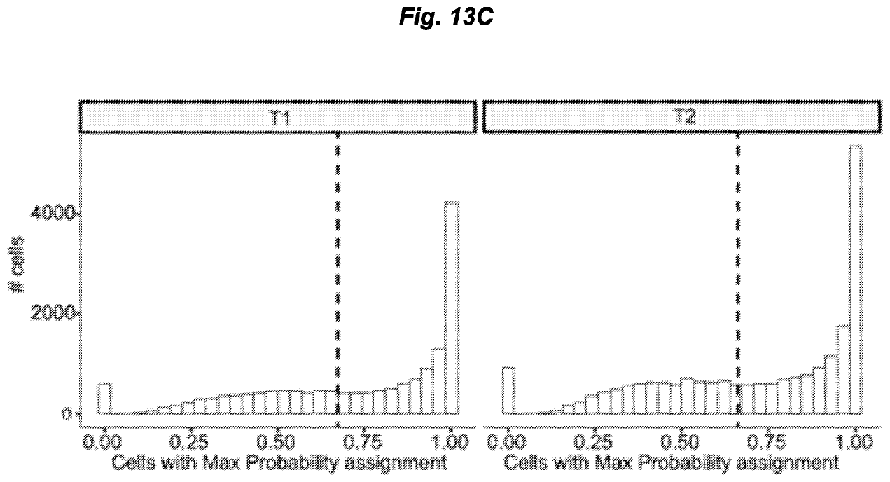

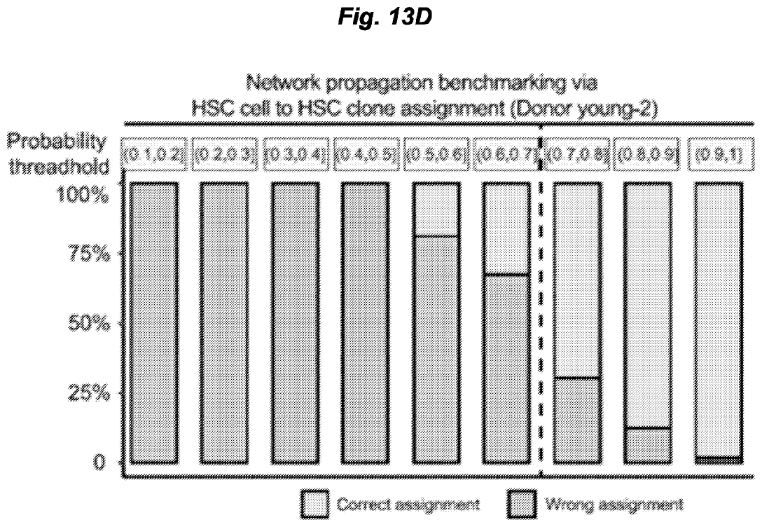

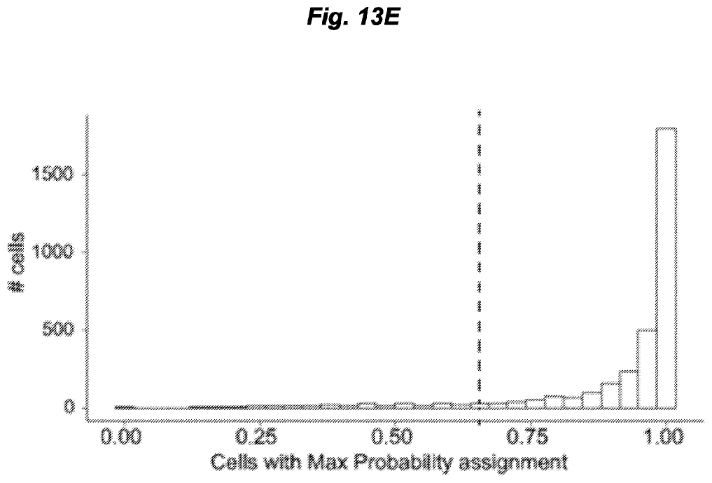

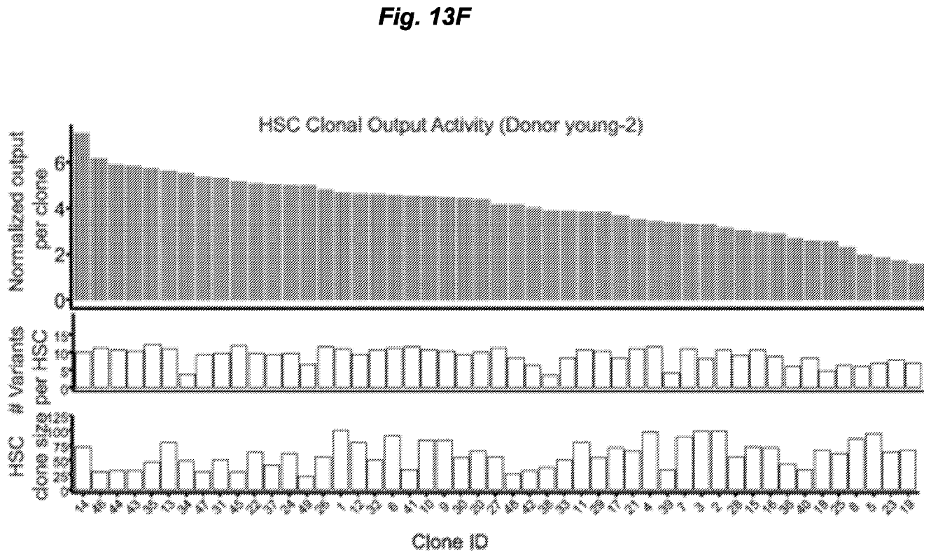

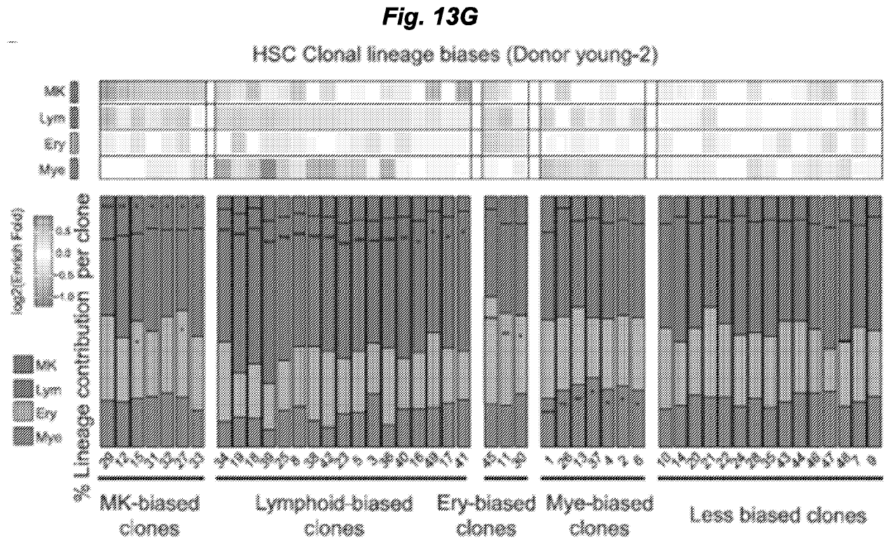

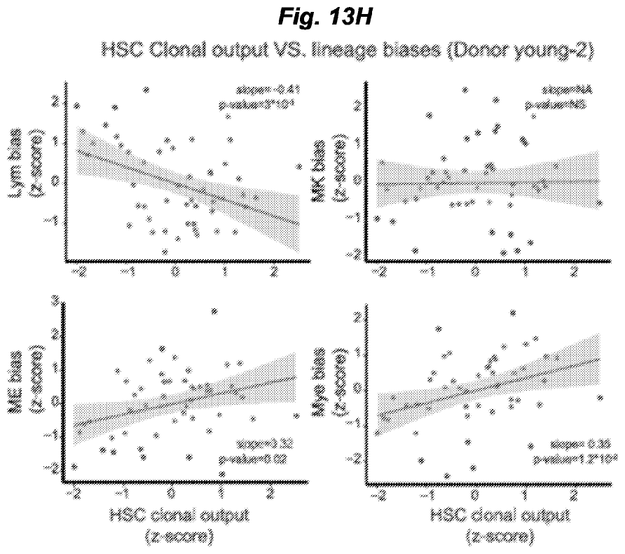

Traditionally, the functional output of HSCs could only be measured in transplantation settings or with barcoding in model systems, which cannot be applied to native human hematopoiesis. Given the advances in detecting deep mtDNA mutations as natural cellular barcodes, such tracking is now possible. To avoid confounding from dropout or detection failure of specific mtDNA mutations, SCAVENGE-L, a computational method based on a network propagation strategy that maximally utilises informative mtDNA mutations to identify the progeny of distinct HSC clones⁶¹ was developed (FIG. 4a, Methods). Briefly, an inclusive clonal network is first built for all cells from the same donor (including HSCs, as well as committed progenitors and differentiated cells) using shared high fidelity mtDNA mutations. Next, the cells of each HSC clonal group serve as seeds and propagate the clonal information through the network until a stationary state is reached. Each clonal group is used for network propagation iteratively. The information carried by each cell in the terminal state represents the probability that this cell is the progeny of the assessed HSC clonal group and normalised scores are compared across all clonal groups to determine the assignment. With SCAVENGE-L analysis, most cells can be mapped to one distinct HSC clonal group with an exclusively high probability of assignment (FIG. 13a). To further benchmark the accuracy of this approach, the network propagation-based assignment was compared with the originally identified HSC clonal groups (ground truth). As expected, the accuracy of the assignment increases for the cells with higher maximum assignment probability. Cells were then filtered with the maximum probability of 0.7, by which more than 80% of the HSCs can be assigned to the correct HSC clonal group successfully (FIG. 13b, Methods). Collectively, the benchmarking analyses at both time points across both donors demonstrate the robustness and consistency of SCAVENGE-L (FIG. 13b-e).

The extent to which HSC output variation and lineage biases exist is controversial and most prior studies have relied on labelling in mouse models and/or transplantation assays^{5,16-20}. In donor young-1, 22,349 (or 59%) committed and differentiated cells were confidently assigned to the HSC clonal groups with the highest assignment probability greater than 0.7 (via SCAVENGE-L). The output of a specific clone to differentiated blood and immune cells can then be directly measured and compared across different HSC clonal groups after normalising for clone size (FIG. 4b, Methods). All HSC clonal groups are actively producing progeny in young-1, but there is some variation in the extent of output between clones, with 4.9-fold differences in output between the top and bottom clonal deciles (FIG. 4b). Interestingly, this variable output activity shows high consistency at the clonal level between the two time points spanning 4 months (Pearson's r=0.69) (FIG. 4b,c). As expected, in donor young-2, the HSC clonal output activity also shows variability with a similar pattern (with 4.5-fold change between top and bottom clonal deciles, FIG. 13f). The overall HSC clonal contributions in hematopoiesis was further quantified and it was found that the top 50% of HSC clones based on output give rise to approximately only 60% of mature hematopoietic cells in two time points and across both donors (FIG. 4d). These results suggest that most HSC clones actively contribute to human steady-state hematopoiesis, but some sustainable variability over many months is observed between HSCs.

The extent to which HSCs display lineage bias in native human hematopoiesis is unclear. This data allows us to investigate the cell states of progeny assigned to different HSC clonal groups. 4 main lineages are defined by grouping cell states based on the multi-omic data: myeloid (monocytes, GMP, MDP, cDC), lymphoid (CD4, CD8, NK, B, ProB, CLP), erythroid (MEP, EryP), and megakaryocyte (MKP) lineages (FIG. 2c). The lineage contribution for each HSC clonal group is then computed. In young-1, Compared to the expected lineage distribution using all cells, 47 (60%) HSC clonal groups were identified with significant lineage enrichment (binomial test pt1<0.05 & pt2<0.05), while 31 (40%) HSC clones showed low or no lineage bias (FIG. 4e). Notably, the lineage preference of biased clones shows only a moderate effect size (with a median 1.55-fold change), but is highly reproducible across two time points spanning several months (Pearson's r=0.59). Consistently, 69% of lineage-biased HSC clones and 31% of unbiased HSC clones in young-2 were observed (FIG. 13g). Next, the relationships between the clonal output and the lineage preference across HSC clonal groups was explored Interestingly, that lymphoid lineage bias is negatively correlated with HSC clonal output; erythroid and myeloid lineages are positively correlated with HSC clonal output, and MK lineages show no significant difference (FIG. 4). This is consistent across two donors as well as with findings using orthogonal approaches from prior reports¹⁶. Taken together, these results suggest that HSCs have moderate, but relatively stable lineage biases across time in native human hematopoiesis.

### Oligoclonal Hematopoietic Expansions in Aged Individuals

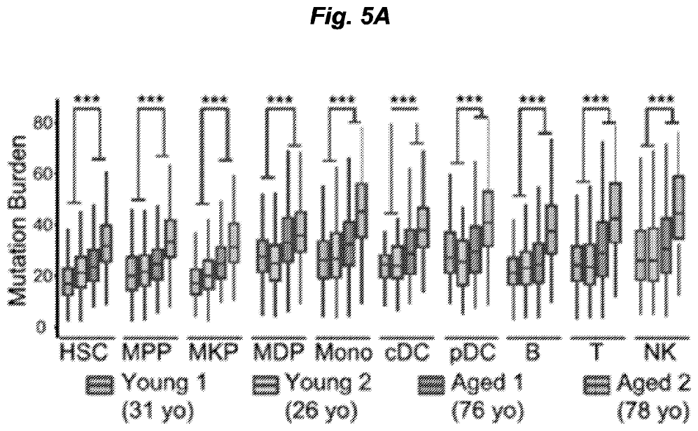

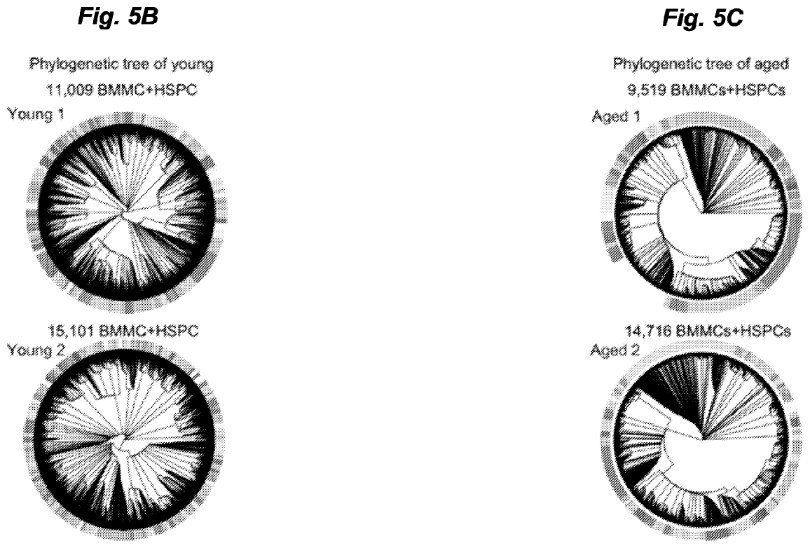

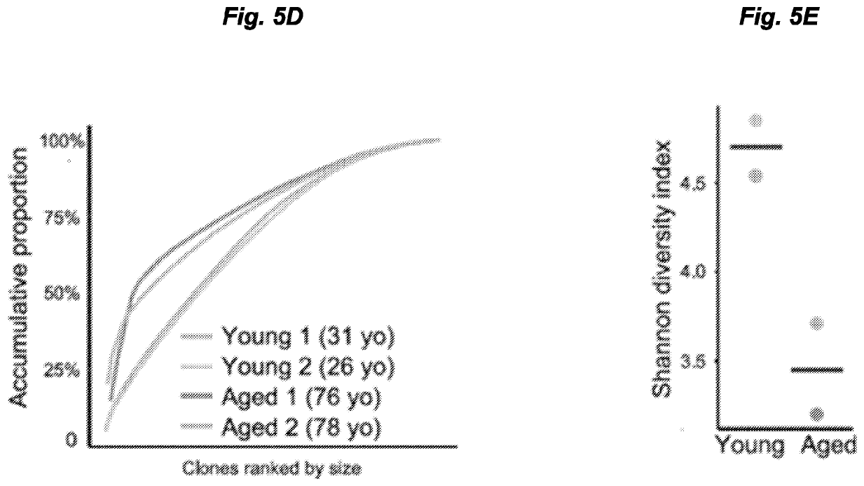

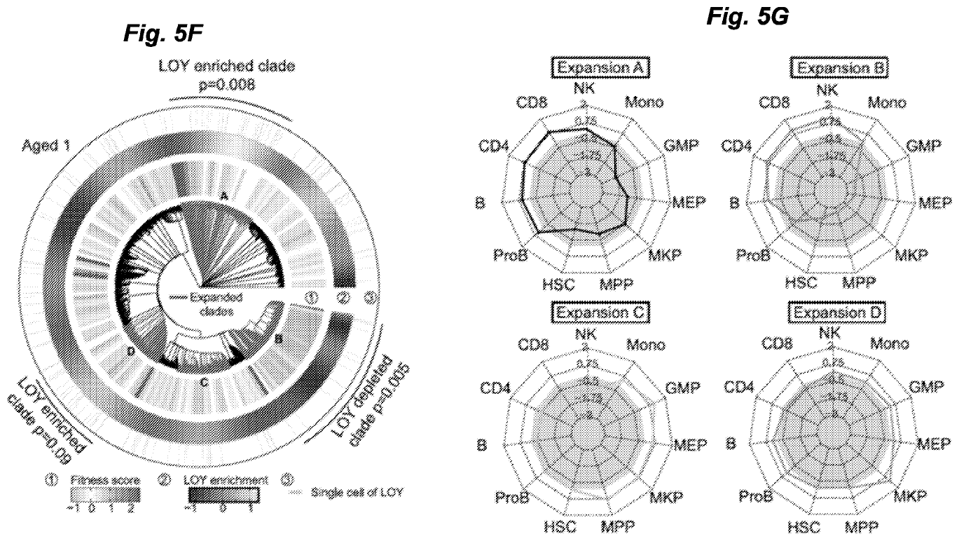

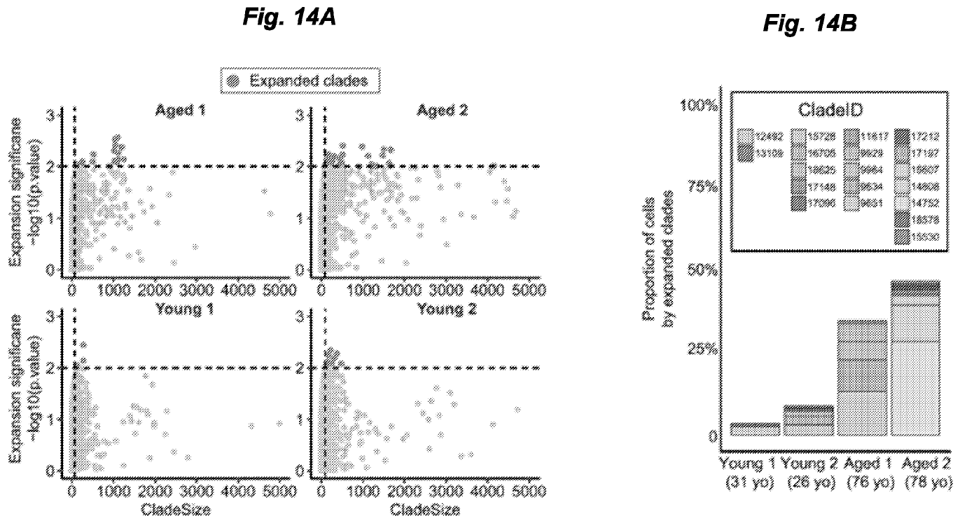

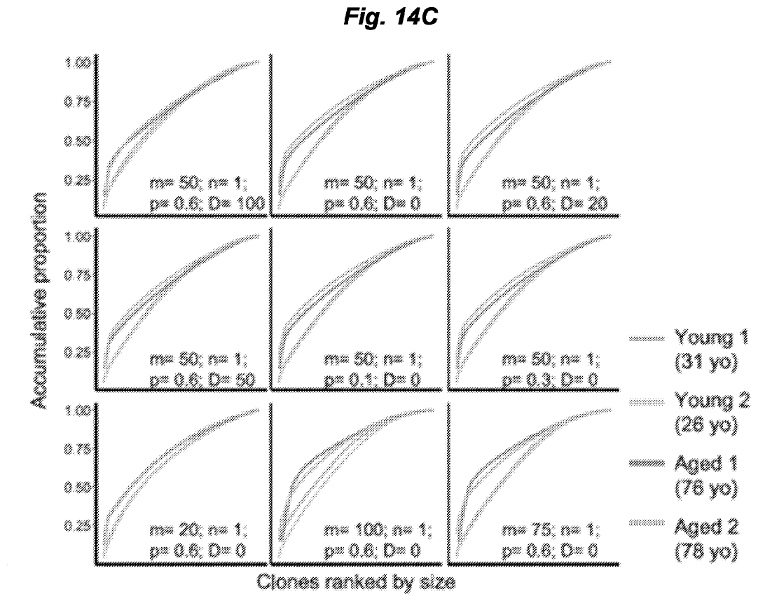

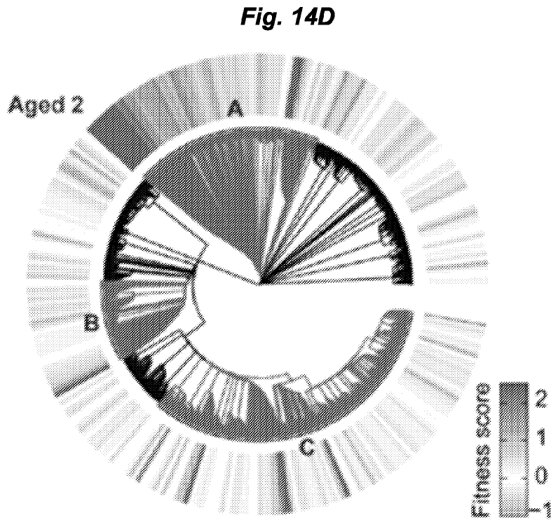

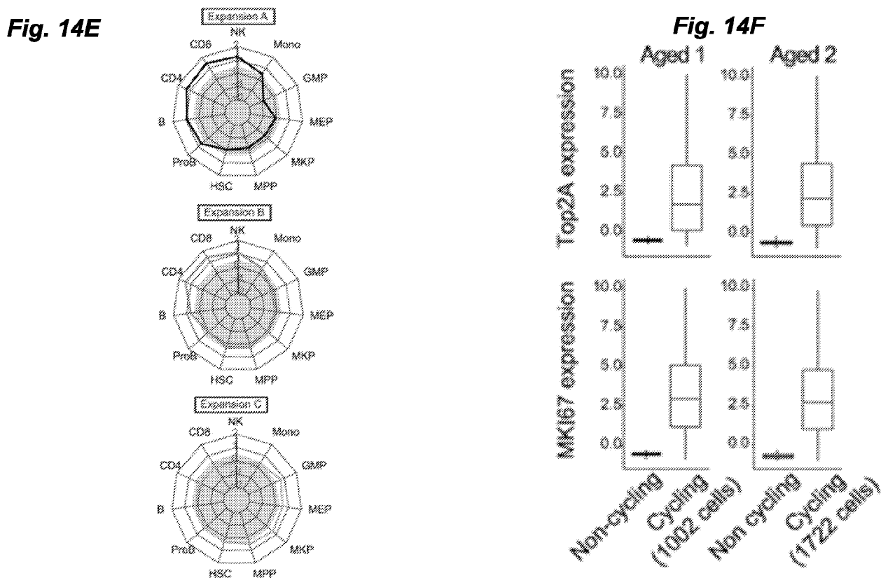

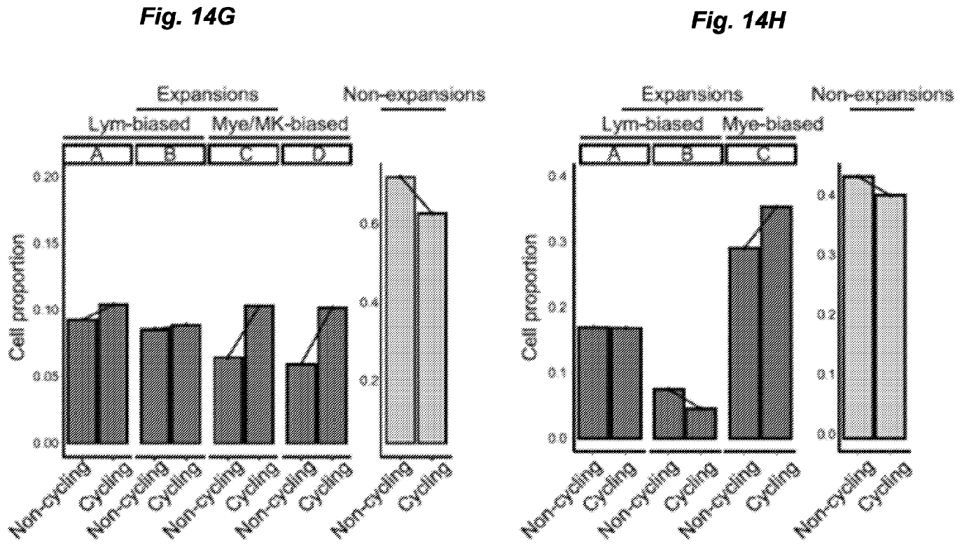

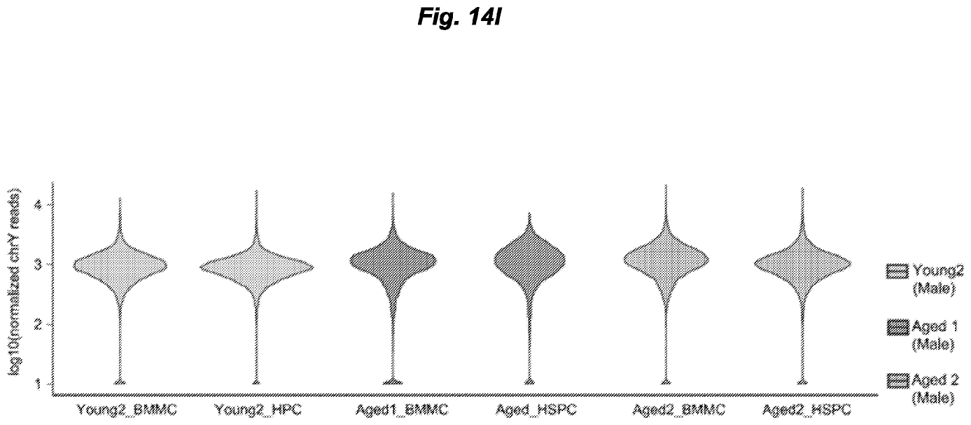

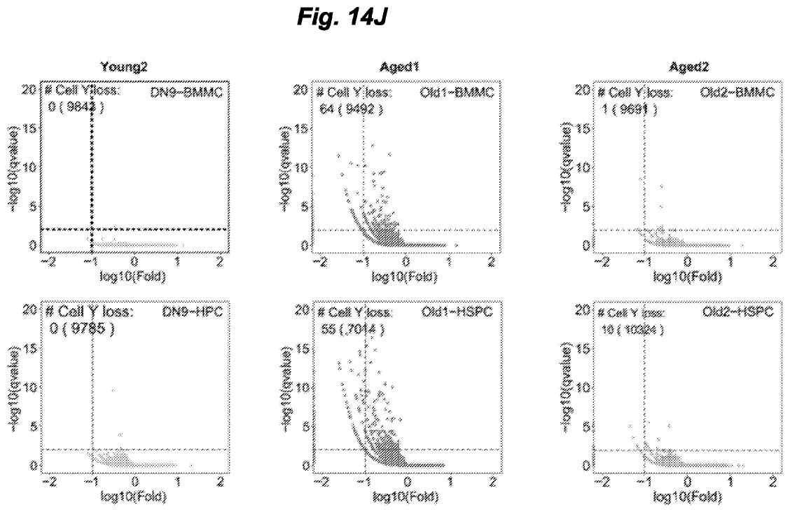

Recent studies have suggested that there is both attrition of HSCs with age and expansion of specific clones that harbour disease driver mutations, which can increase the risk for acquiring leukaemia and other morbidities, a phenomenon termed clonal hematopoiesis^{7,62}. However, most detection of such clonal expansions has relied upon the use of specific driver mutations with bulk sequencing methods and the extent of clonal complexity that would be observed at single-cell resolution remains unstudied. It was reasoned that the newly developed multi-omic approach that enables detection of natural cellular barcodes can enable new insights into aged hematopoiesis. The approach was applied to profile 9,519 and 14,715 hematopoietic cells on two donors, aged 76 and 78 years, which were termed aged-1 and aged-2 below. Significantly increased mtDNA mutation burden were detected in the aged donors across all the identified cell types, consistent with reports from somatic mutations in nuclear genomes^{26,63} (FIG. 5a, Methods). Based on shared mtDNA mutations, the phylogenetic tree was reconstructed for each aged donor. Remarkably, the resulting trees exhibit substantially more oligoclonal structure compared to that of young donors (FIG. 5b,c). 48 and 84 clonal groups were identified for aged-1 and aged-2 by cutting down the phylogenetic tree structure using the same methods (Methods). The aged donors had a few large clones that dominated the architecture of hematopoiesis, with lower clonal diversity (Shannon Index), compared to the young donors (FIG. 5d,e, FIG. 14c, Methods). To examine sub-clonal dynamics, a statistical test was adapted to quantify the relative clade size to those expected if HSCs were evolving under a neutral evolution model⁴². Multiple expanded clades were identified in the aged donors (with the size over 500 cells that are under positive selection with p value<0.01), which are nearly absent in the young donors (FIG. 14a,b). The proportion of cells in expanded clades are 34.4% in aged-1, 46.3% in aged-2, whereas only 3.4% in young-1 and 8.7% in young-2. Next, the “fitness score”, which is defined as the growth advantage compared to the rest of the population, was inferred for every single cell in aged donors^{42,64,65} (Methods). This analysis reveals the variation of single-cell fitness within the same donor. As expected, the cells in expanded clades showed high fitness scores (FIG. 5f, FIG. 14d).

Hematopoietic mosaic loss of Y chromosome (mLOY) is frequently observed with ageing in men and is associated with a number of morbidities. However, the causes and consequences of mLOY are still unclear⁶⁶. Here, based on the single-cell ATAC fragments on chromosome Y, quantitative metrics were developed to estimate LOY in single cells (Methods). 119 and 11 cells were identified with LOY in aged-1 and aged-2, in contrast, LOY cells in young male donors were not identified (FIG. 14i,j). For aged-1, the identity of cells with or without LOY were mapped on the phylogenetic tree. The LOY cells appear in multiple branches but are most significantly enriched in expanded clade A that shows the highest fitness scores. Interestingly, other expansions were also identified, such as expanded clade B that shows significant LOY cell depletion and therefore likely has a different driver mutation (FIG. 5f). These results suggest that the LOY likely occurs multiple times in parallel in this donor and is enriched in cells with higher fitness scores, reminiscent of prior reports²⁶. Finally, the cell type composition were investigated within each expanded clade, which is enabled by the joint multi-omic readouts available through this method. Different expanded clades show skewed cell type distributions in both aged donors (FIG. 5g, FIG. 14e). Interestingly, the expanded clade A that shows enrichment for LOY is biased towards the lymphoid lineage, which echoes the recent analysis using bulk data from the UK Biobank showing a strong correlation between LOY and individual lymphocyte counts⁵². Collectively, these results reshape the view of aged hematopoiesis and rather than detecting a single clonal expansion, as is typically thought to occur with clonal hematopoiesis from bulk sequencing studies, a more complex oligoclonal architecture was detected with detailed cell states.

### Discussion

The study of human hematopoiesis has served as a paradigm for the understanding of stem cell biology and regenerative medicine. Despite decades of effort, central questions in human hematopoiesis remain unresolved⁵⁹⁻⁶¹. For example, the extent to which ‘clonal succession’ (only few stem cells would contribute) versus ‘clonal stability’ (many stem cells contribute simultaneously)^{67,68} models best describe native hematopoiesis is unclear, as is the extent to which unperturbed populations of HSCs have restricted differentiation potency or lineage biases^{2,69}. Various transplantation-based assays as well as cell-labelling based methods have provided important insights but with respective limitations, especially for the exploration of these problems in a native human context^{5,13,70,71}.

Presented here is a high resolution, engineering-free massively parallel single-cell lineage tracing approach that can be directly applied to human samples. Using this approach, provided here is a clonally-resolved and cell-state aware single cell atlas for native human hematopoiesis and use this atlas to explore the clonal architecture and the heterogeneous behaviour of human HSCs at steady state in vivo. It is shown that in young individuals, the majority of HSC clones are indeed contributing to hematopoiesis at steady state, but with some (5-fold) differences in clone-specific output activity, and that these differences are stably maintained over the several month timescale. Further demonstrated here is that there are inherent clone-specific lineage biases that like the clonal differences in output are confined in magnitude but sustained across time. Finally, HSC subpopulations are identified using joint transcriptomic and epigenomic states and found that a notable subset of HSC clonal groups are enriched in certain HSC subpopulations defined by gene expression and epigenomic landscapes. Interestingly, the HSC clone-specific cell-state preference in human native context is also an inherent feature that is relatively stable, which echoes some labelling-based mouse studies^{5,72}.

Thus, together with previous studies, a picture of normal hematopoiesis emerges from this work in which in young individuals there is a rich and balanced polyclonal architecture for HSCs contributions to hematopoiesis, with each subclone having distinct but confined preferences in cell state, output and lineage biases. By contrast, in elderly individuals there is a marked breakdown in this clonal diversity. Clonal expansion or the alteration of clonal diversity is involved in various cancers and premalignant conditions. Clonal hematopoiesis has been identified as highly prevalent in elderly and substantially increases the risk of leukaemia and cardiovascular diseases^{6,62}. However, the causes and consequences of the diminished clonal diversity are largely unknown and difficult to study in humans. These results suggest that the clonal expansions can arise with multiple origins and different lineage biases. The ability to capture and characterise clonal expansions at single-cell resolution in ageing permits the in depth exploration of the molecular nature of these expanding clones.

More broadly, somatic mutations have increasingly been found to contribute to a variety of disease processes beyond hematopoiesis and cancer⁷³. Compared to single-colony whole genome sequencing, this cell state-aware single-cell lineage tracing technology contains rich cell state information, with high scalability and dramatically lower cost per cell, which makes it possible to broadly explore the extent and nature of such subclonal changes with applications to broad areas of human research in health and disease.

### REFERENCES

1. Orkin, S. H. & Zon, L. I. Hematopoiesis: an evolving paradigm for stem cell biology. Cell 132, 631-644 (2008).
2. Haas, S., Trumpp, A. & Milsom, M. D. Causes and Consequences of Hematopoietic Stem Cell Heterogeneity. Cell Stem Cell22, 627-638 (2018).
3. Muller-Sieburg, C. E., Sieburg, H. B., Bernitz, J. M. & Cattarossi, G. Stem cell heterogeneity: implications for aging and regenerative medicine. Blood 119, 3900-3907 (2012).
4. Lu, R., Czechowicz, A., Seita, J., Jiang, D. & Weissman, I. L. Clonal-level lineage commitment pathways of hematopoietic stem cells in vivo. Proc. Natl. Acad. Sci. U.S.A. 116, 1447-1456 (2019).
5. Yu, V. W. C. et al. Epigenetic Memory Underlies Cell-Autonomous Heterogeneous Behavior of Hematopoietic Stem Cells. Cell vol. 167 1310-1322.e17 Preprint (2016).
6. Jaiswal, S. & Ebert, B. L. Clonal hematopoiesis in human aging and disease. Science 366, (2019).
7. Jaiswal, S. et al. Age-related clonal hematopoiesis associated with adverse outcomes. N. Engl. J. Med. 371, 2488-2498 (2014).
8. Mead, A. J. & Mullally, A. Myeloproliferative neoplasm stem cells. Blood 129, 1607-1616 (2017).
9. Yamamoto, R. et al. Clonal analysis unveils self-renewing lineage-restricted progenitors generated directly from hematopoietic stem cells. Cell 154, 1112-1126 (2013).
10. Sanjuan-Pla, A. et al. Platelet-biased stem cells reside at the apex of the haematopoietic stem-cell hierarchy. Nature 502, 232-236 (2013).
11. Weinreb, C., Rodriguez-Fraticelli, A., Camargo, F. D. & Klein, A. M. Lineage tracing on transcriptional landscapes links state to fate during differentiation. Science 367, (2020).
12. Patel, S. H. et al. Lifelong multilineage contribution by embryonic-born blood progenitors. Nature 606, 747-753 (2022).
13. Rodriguez-Fraticelli, A. E. et al. Single-cell lineage tracing unveils a role for TCF15 in haematopoiesis. Nature 583, 585-589 (2020).
14. Bowling, S. et al. An Engineered CRISPR-Cas9 Mouse Line for Simultaneous Readout of Lineage Histories and Gene Expression Profiles in Single Cells. Cell 181, 1410-1422.e27 (2020).
15. Sharma, R. et al. The TRACE-Seq method tracks recombination alleles and identifies clonal reconstitution dynamics of gene targeted human hematopoietic stem cells. Nat. Commun. 12, 472 (2021).
16. Chapple, R. H. et al. Lineage tracing of murine adult hematopoietic stem cells reveals active contribution to steady-state hematopoiesis. Blood Adv 2, 1220-1228 (2018).
17. Sun, J. et al. Clonal dynamics of native haematopoiesis. Nature 514, 322-327 (2014).
18. Busch, K. et al. Fundamental properties of unperturbed haematopoiesis from stem cells in vivo. Nature vol. 518 542-546 Preprint (2015).
19. Pei, W. et al. Polylox barcoding reveals haematopoietic stem cell fates realized in vivo. Nature 548, 456-460 (2017).
20. Sawai, C. M. et al. Hematopoietic Stem Cells Are the Major Source of Multilineage Hematopoiesis in Adult Animals. Immunity 45, 597-609 (2016).
21. Biasco, L. et al. In Vivo Tracking of Human Hematopoiesis Reveals Patterns of Clonal Dynamics during Early and Steady-State Reconstitution Phases. Cell Stem Cell 19, 107-119(2016).
22. Lee-Six, H. et al. Population dynamics of normal human blood inferred from somatic mutations. Nature 561, 473-478 (2018).
23. Coorens, T. H. H. et al. Extensive phylogenies of human development inferred from somatic mutations. Nature 597, 387-392 (2021).
24. Park, S. et al. Clonal dynamics in early human embryogenesis inferred from somatic mutation. Nature 597, 393-397 (2021).
25. Spencer Chapman, M. et al. Lineage tracing of human development through somatic mutations. Nature 595, 85-90 (2021).
26. Mitchell, E. et al. Clonal dynamics of haematopoiesis across the human lifespan. Nature 606, 343-350 (2022).
27. Williams, N. et al. Life histories of myeloproliferative neoplasms inferred from phylogenies. Nature 602, 162-168 (2022).
28. Ludwig, L. S. et al. Lineage Tracing in Humans Enabled by Mitochondrial Mutations and Single-Cell Genomics. Cell 176, 1325-1339.e22 (2019).
29. Lareau, C. A. et al. Massively parallel single-cell mitochondrial DNA genotyping and chromatin profiling. Nat. Biotechnol. 39, 451-461 (2021).
30. Mimitou, E. P. et al. Scalable, multimodal profiling of chromatin accessibility, gene expression and protein levels in single cells. Nat. Biotechnol. 39, 1246-1258 (2021).
31. Xu, J. et al. Single-cell lineage tracing by endogenous mutations enriched in transposase accessible mitochondrial DNA. Elife 8, (2019).
32. Miller, T. E. et al. Mitochondrial variant enrichment from high-throughput single-cell RNA sequencing resolves clonal populations. Nat. Biotechnol. 40, 1030-1034 (2022).
33. Kang, E. et al. Age-Related Accumulation of Somatic Mitochondrial DNA Mutations in Adult-Derived Human iPSCs. Cell Stem Cell 18, 625-636 (2016).
34. Wallace, D. C. Mitochondrial DNA sequence variation in human evolution and disease. Proc. Natl. Acad. Sci. U.S.A. 91, 8739-8746 (1994).
35. Kwok, A. W. C. et al. MQuad enables clonal substructure discovery using single cell mitochondrial variants. Nat. Commun. 13, 1205 (2022).
36. Schmitt, M. W. et al. Detection of ultra-rare mutations by next-generation sequencing. Proc. Natl. Acad. Sci. U.S.A. 109, 14508-14513 (2012).
37. Kennedy, S. R. et al. Detecting ultralow-frequency mutations by Duplex Sequencing. Nat. Protoc. 9, 2586-2606 (2014).
38. Hoang, M. L. et al. Genome-wide quantification of rare somatic mutations in normal human tissues using massively parallel sequencing. Proc. Natl. Acad. Sci. U.S.A. 113, 9846-9851 (2016).
39. Abascal, F. et al. Somatic mutation landscapes at single-molecule resolution. Nature 593, 405-410 (2021).
40. Salk, J. J., Schmitt, M. W. & Loeb, L. A. Enhancing the accuracy of next-generation sequencing for detecting rare and subclonal mutations. Nat. Rev. Genet. 19, 269-285 (2018).
41. Ju, Y. S. et al. Origins and functional consequences of somatic mitochondrial DNA mutations in human cancer. Elife 3, (2014).
42. Yang, D. et al. Lineage tracing reveals the phylodynamics, plasticity, and paths of tumor evolution. Cell 185, 1905-1923.e25 (2022).
43. Hao, Y. et al. Integrated analysis of multimodal single-cell data. Cell 184, 3573-3587.e29 (2021).
44. Nerlov, C., Querfurth, E., Kulessa, H. & Graf, T. GATA-1 interacts with the myeloid PU.1 transcription factor and represses PU.1-dependent transcription. Blood95, 2543-2551 (2000).
45. Qiu, X. et al. Mapping transcriptomic vector fields of single cells. Cell 185, 690-711.e45 (2022).
46. Anderson, D. A., 3rd, Dutertre, C.-A., Ginhoux, F. & Murphy, K. M. Genetic models of human and mouse dendritic cell development and function. Nat. Rev. Immunol. 21, 101-115(2021).
47. Feng, J. et al. Clonal lineage tracing reveals shared origin of conventional and plasmacytoid dendritic cells. Immunity 55, 405-422.e11 (2022).
48. Liggett, L. A. & Sankaran, V. G. Unraveling Hematopoiesis through the Lens of Genomics. Cell 182, 1384-1400 (2020).
49. Ziegler-Heitbrock, L., Ohteki, T., Ginhoux, F., Shortman, K. & Spits, H. Reclassifying plasmacytoid dendritic cells as innate lymphocytes. Nat. Rev. Immunol. (2022) doi:10.1038/s41577-022-00806-0.
50. Musumeci, A., Lutz, K., Winheim, E. & Krug, A. B. What Makes a pDC: Recent Advances in Understanding Plasmacytoid DC Development and Heterogeneity. Front. Immunol. 10, 1222 (2019).
51. Merad, M., Sathe, P., Helft, J., Miller, J. & Mortha, A. The dendritic cell lineage: ontogeny and function of dendritic cells and their subsets in the steady state and the inflamed setting. Annu. Rev. Immunol. 31, 563-604 (2013).
52. Voit, R. A. et al. A genetic disorder reveals a hematopoietic stem cell regulatory network co-opted in leukemia. bioRxiv 2021.12.09.471942 (2022) doi:10.1101/2021.12.09.471942.
53. Bao, E. L. et al. Inherited myeloproliferative neoplasm risk affects haematopoietic stem cells. Nature 586, 769-775 (2020).
54. Calvanese, V. et al. MLLT3 governs human haematopoietic stem-cell self-renewal and engraftment. Nature 576, 281-286 (2019).
55. Lin, C.-C. et al. Knock-out of Hopx disrupts sternness and quiescence of hematopoietic stem cells in mice. Oncogene 39, 5112-5123 (2020).
56. Scheicher, R. et al. CDK6 as a key regulator of hematopoietic and leukemic stem cell activation. Blood 125, 90-101 (2015).
57. Kennedy, V. E. & Smith, C. C. FLT3 Mutations in Acute Myeloid Leukemia: Key Concepts and Emerging Controversies. Front. Oncol. 10, 612880 (2020).
58. Laurenti, E. et al. CDK6 levels regulate quiescence exit in human hematopoietic stem cells. Cell Stem Cell 16, 302-313 (2015).
59. Crisan, M. et al. BMP signalling differentially regulates distinct haematopoietic stem cell types. Nat. Commun. 6, 8040 (2015).
60. Fast, E. M. et al. External signals regulate continuous transcriptional states in hematopoietic stem cells. Elife 10, (2021).
61. Yu, F. et al. Variant to function mapping at single-cell resolution through network propagation. Nat. Biotechnol. 40, 1644-1653 (2022).
62. Jaiswal, S. et al. Clonal Hematopoiesis and Risk of Atherosclerotic Cardiovascular Disease. N. Engl. J. Med 377, 111-121 (2017).
63. Machado, H. E. et al. Diverse mutational landscapes in human lymphocytes. Nature 608, 724-732(2022).
64. Williams, M. J. et al. Quantification of subclonal selection in cancer from bulk sequencing data. Nat. Genet. 50, 895-903 (2018).
65. Neher, R. A., Russell, C. A. & Shraiman, B. I. Predicting evolution from the shape of genealogical trees. Elife 3, (2014).
66. Thompson, D. J. et al. Genetic predisposition to mosaic Y chromosome loss in blood. Nature 575, 652-657 (2019).
67. Bystrykh, L. V., Verovskaya, E., Zwart, E., Broekhuis, M. & de Haan, G. Counting stem cells: methodological constraints. Nat. Methods 9, 567-574 (2012).
68. McKenzie, J. L., Gan, O. I., Doedens, M., Wang, J. C. Y. & Dick, J. E. Individual stem cells with highly variable proliferation and self-renewal properties comprise the human hematopoietic stem cell compartment. Nat. Immunol. 7, 1225-1233 (2006).
69. Jurecic, R. Hematopoietic Stem Cell Heterogeneity. Adv. Exp. Med. Biol. 1169, 195-211 (2019).
70. Pei, W. et al. Resolving fate and transcriptome of hematopoietic stem cell clones. bioRxiv 2020.03.25.008433 (2020) doi:10.1101/2020.03.25.008433.
71. Carrelha, J. et al. Hierarchically related lineage-restricted fates of multipotent haematopoietic stem cells. Nature 554, 106-111 (2018).
72. Bernitz, J. M., Kim, H. S., MacArthur, B., Sieburg, H. & Moore, K. Hematopoietic Stem Cells Count and Remember Self-Renewal Divisions. Cell 167, 1296-1309.e10 (2016).
73. Sankaran, V. G., Weissman, J. S. & Zon, L. I. Cellular barcoding to decipher clonal dynamics in disease. Science 378, eabm5874 (2022).
74. Rausser, S. et al. Mitochondrial phenotypes in purified human immune cell subtypes and cell mixtures. Elife 10, (2021).
75. Laricchia, K. M. et al. Mitochondrial DNA variation across 56,434 individuals in gnomAD. Genome Res. 32, 569-582 (2022).
76. Jones, M. G. et al. Inference of single-cell phylogenies from lineage tracing data using Cassiopeia. Genome Biol. 21, 92 (2020).
77. van Dijk, D. et al. Recovering Gene Interactions from Single-Cell Data Using Data Diffusion. Cell 174, 716-729.e27 (2018).

### Methods

### Bone Marrow Donors

The Fresh bone marrow samples from healthy young donors were aspirated with informed consent under a sample banking protocol that was approved by Institutional Review Board (IRB) of Boston Children's Hospital. The sternum bone marrow from aged donors was collected following sternotomy for cardiac surgery with informed consent under a sample banking protocol that was approved by the IRB of Mass General Brigham. The individual donor information is shown below:

| | | | Samples | Longitudinal |
|---|---|---|---|---|
| Donor ID | Age | Gender | profiled | sampling |
| Young-1 | 31 | Female | BMMC, CD34⁺, | Twice with 4 |
| | | | CD34⁺CD45RA | months apart |
| | | | CD90⁺ | |
| Young-2 | 26 | Male | BMMC, CD34⁺, | Once |
| | | | CD34⁺CD45RA | |
| | | | CD90⁺ | |
| Aged-1 | 76 | Male | BMMC, CD34⁺ | Once |
| Aged-2 | 78 | Male | BMMC, CD34⁺ | Once |

Note:
BMMCs indicate extracted bone marrow mononuclear cells, CD34⁺ cells are enriched cells for hematopoietic stem and progenitor cells (HSPCs), CD34⁺CD45RA CD90⁺ are enriched cells for hematopoietic stem cells (HSCs, see below for more details.

### Primary Bone Marrow Mononuclear Cells (BMMSs) Extraction

Bone marrow was collected from healthy young or aged donors (Individual information is shown above). Bone marrow aspirate was diluted with an equal volume of Wash Buffer (PBS, 2% FBS, 1 mM EDTA). Ficoll medium was added to SepMate™ (Catalog #85460) tubes and then the diluted bone marrow sample was layered on top followed by centrifuging at 1200 g for 20 minutes at room temperature. The top layer, containing the mononuclear cells, was transferred to a new tube, which was then filled up by Wash Buffer. The mononuclear cells were centrifuged at 300 g for 8 minutes. The supernatant was discarded and the cells were washed twice and resuspended in Wash Buffer for further enrichment or in Freezing buffer (10% DMSO in FBS).

### Enrichment for Hematopoietic Stem and Progenitor Cells

Starting with the BMMCs isolated from the previous step, CD34+ cells are enriched following EasySep™ Human Cord Blood CD34 Positive Selection Kit II (Catalog #17896). Briefly, EasySep™ Human CD34 Positive Selection Cocktail (Catalog #18096C) was added to BMMCs suspension to 100 ul/ml and incubated at room temperature for 10 minutes. EasySep™ Dextran RapidSpheres™ (Catalog #50100) was vortexed and added to each sample to 50 μl/ml. The mix was incubated for 3 minutes at room temperature. Next, Wash Buffer (7 ml) was added to the tube, The cells were then washed 4 times in “The Big Easy” EasySep™ Magnet (Catalog #18001). Finally, cells were resuspended in Wash Buffer and were centrifuged at 300 g for 10 min. The CD34⁺ positive cell pellet was then resuspended in a freezing buffer (10% DMSO in FBS).

To further enrich the hematopoietic stem cells (HSCs), an aliquot of the enriched CD34⁺ cells were stained by one of the following two antibody panels. 1) CD34 PerCP-Cy5.5 (Catalog #347222); CD45RA Alexa Fluor 488 (Catalog #304114); CD90 PE-Cy7 (Catalog #561558); and DAPI (Catalog #D1306) as viability dye. Or 2) CD34 BV421 (Catalog #562577); CD45RA-APC-H7 (Catalog #560674); CD90 PE-Cy7 (Catalog #561558), and 7-AAD as viability dye (Catalog #559925). The cells were further sorted using BD FACSAria™ for CD34⁺CD45RA⁻ CD90⁺ to enrich HSCs.

BMMCs, as well as enriched CD34⁺ and CD34⁺CD45RA⁻CD90⁺ cells were cryopreserved. After thawing, cells were immediately processed for experiment as soon as possible without any culturing.

### Principle of ReDeeM

Developed here is single-cell Regulatory multi-omics with Deep Mitochondrial mutation profiling (ReDeeM), which is a modified massive parallel single cell protocol to simultaneously profile multi-omics with deep mtDNA sequencing based on the 10× Genomics platform. With ReDeeM, three separate libraries are generated, including enriched mtDNA library for deep sequencing and mutation profiling, RNA library for gene expression, as well as ATAC library for chromatin accessibility profiling, all of which are linked via matchable single cell barcodes.

The droplet-based 10× genomics multi-omics protocol (Catalog #100283) is firstly modified by processing the whole cell instead of nuclei with fixation and mild permeabilization to maximally retain mitochondrial DNA. Next, mtDNA specific probe sets are designed to enrich the mitochondrial fragments using DNA hybridization. The RNA and ATAC library preps follow the standard 10× protocol with some modifications.

More method details are described in the following sections. See ReDeeM Protocol. ReDeeM is further computationally supported by consensus variant calling pipeline redeemV, and an inhouse R package redeemR for the downstream mutation quality control, as well as single cell phylogenetic and integrative analysis.

### ReDeeM Protocol

The detailed protocol is found in example 2.

### Cell Fixation, Permeabilization and Tagmentation

A modified fixation and permeabilization approach greatly improved the access to mtDNA in whole fixed cells, and thereby facilitated a dramatic improvement in detecting mtDNA variations. For each sample, cells were resuspended in 0.1% formaldehyde in DPBS, with 0.04% bovine serum albumin (BSA) and 0.2 U/μl RNase Inhibitor (RI), and incubated at room temperature for 10 minutes. The fixation was then quenched by 0.125M glycine, at room temperature for 3 minutes. Cells were centrifuged 500 g for 5 min at 4 C and washed once by 3 ml cold PBS/RI (DPBS with 0.04% BSA, 0.2 U/ul RI). After a second centrifugation and removal of supernatant, cells were incubated for 5 minutes on ice in 1 ml permeabilization buffer (10 mM Tris, 10 mM NaCl, 3 mM MgCl₂, 0.1% NP40, 1 mM DTT, 1 U/μl RI), which were followed by adding 3 ml cold Wash buffer (10 mM Tris, 10 mM NaCl, 3 mM MgCl₂, 0.02% BSA, 1 mM DTT, 0.2 U/ul RI). Cells were then centrifuged and resuspended in 1× Nuclei Buffer (10× Genomics, Catalog #PN2000207). Cells were counted and diluted to 2,800 cells/μl, which served as the input for tagmentation reaction. A modified tagmentation protocol was applied as follows, a volume of 5 μl of cell suspension from previous step was mixed with 5.5 μl ATAC buffer B (10× Genomics, Catalog #PN2000193), 1.5 μl ATAC-GDN (ATAC buffer B with 1% glyco-diosgenin (or GDN, Avanti Polar Lipids, 850525P-500MG)), and 3 μl Tn5 (ATAC Enzyme B, 10× Genomics, Catalog #PN2000265). The reaction was in 15 ul and incubated at 37° C. for 1 hour and immediately processed to the next step.

### Single-Cell Joint RNA and ATAC Library Preparation

After tagmentation, cells were immediately processed for GEM generation & barcoding following 10× Genomics Chromium Next GEM Single Cell Multiome ATAC+Gene Expression User Manual (version CG000338 Rev A, 10× Genomics) with Step 2. Next, the Post GEM Incubation Cleanup and Pre-Amplification PCR were performed following the same User Manual with Step 3 and Step 4. The pre-amplified library was eluted in 100 ul EB buffer instead of 160 ul.

For RNA library prep, an aliquot of 35 ul pre-amplified library is taken and the same User Manual (version CG000338 Rev A, 10× Genomics) is followed from Step 6 and Step 7.

An in-house protocol was developed to generate the massive ATAC library with enough material for both direct sequencing and mtDNA hybridization. For each sample, 8 reactions of the following were performed (each in 50 ul). 5 pl pre-amplified product, 25 pl NEBnext mix (Catalog M0541L), 2.4 pl 10 pM Nextera N70X primer, 2.4 pl 10× SI PCR primer B (Catalog #PN2000128), 0.5 pl 100×SYBR Green (Catalog #S7563), and 14.7 pl water. The thermocycler program started with 98° C. 45 s., followed by the 6-12 cycles of: 98° C. for 10 s., 63° C. for 30 s., and 72° C. for 20 s.; The actual cycle numbers are determined using the amplification curve and the qPCR was stopped when the curve started to reach plateau. The qPCR products (50 ul*8 wells) were combined and purified using SPRI beads (Catalog #B23318) with size selection 0.4×~1.55×. The expected yield is 2,000 ng to 4,000 ng.

mtDNA Enrichment and Library Preparation

sets of DNA probes were designed, with each probe set covering the whole mitochondrial genome and containing 138 biotinylated oligos that are 120 bp long. The 4 sets of probes ((v1, v2, v3, and v4) are staggered by 30 bp with each other and ensure good coverage on junction areas. The hybridization started with 500 ng ATAC library products for each probe set. In total, 4 independent reactions were performed in parallel and combined at the end (2,000 ng input in total). The detailed incubation and wash methods largely followed the Tube Protocol in IDT xGen Hybridization Capture of DNA Libraries protocol. Briefly, 500 ng input was combined with Cot DNA and purified by SPRI beads, which was eluted by hybridization mix that contains hybridization buffer, hybridization buffer enhancer, one of the probe sets, as well as xGen™ Universal Blockers NXT (Catalog #1079584). The hybridization reaction was performed as below: 95 C 30 seconds followed by 65 C overnight. The biotin-streptavidin Dynabeads purification was carried out on Day 2 using xGen Hybridization and Wash Kit. The Dynabeads after capturing and washing were used for final quick qPCR amplification (3-5 cycles). The reaction mix is as below: 20 ul beads, 25 ul KAPA HiFi HotStart ReadyMix (Catalog #KK2601), Primer 2.4 ul P5, Primer P7, 0.5 ul 100×SYBR Green (Catalog #S7563). The PCR program started with 98° C. 45 s, followed by the 3-5 cycles of: 98° C. for 10 s., 63° C. for 30 s., and 72° C. for 20 s. The final enriched mtDNA was purified by SPRI beads (1.6×).

### Sequencing

For each sample, all three libraries were sequenced on Illumina NovaSeq S4 300 cycle kit. The sequencing length was as below. Read1: 149 nt, i7: 8 nt, i5: 24 nt, Read2: 149 nt. Sequencing 20,000-30,000 raw reads per cell was expected for RNA libraries and ATAC libraries, while 50,000-60,000 raw reads was expected for enrich mtDNA libraries.

Experimental Condition Optimization with Cell Hashing

As mentioned above, to achieve the best mtDNA capture efficiency while maintaining the relatively best data quality of gene expression and chromatin accessibility, a pooled experiment was designed to simultaneously test different conditions of fixation, permeabilization, and tagmentation. The readouts include 1) number of unique mtDNA fragments per cell, 2) number of gene counts per cell. 3) number of unique accessible chromatin fragments per cell. 4) Enrichment of accessible fragments on transcription starting sites (TSS).

To minimise the batch effects and maximise the cost effectiveness of the test experiment, the same pool of cells (BMMCs) was split into 8 aliquots which were cell hashed as previously described and followed by simultaneous test of different conditions. For cell hashing, the procedure was as follows. First, 5 million cells were washed once by DPBS and centrifuged at 500 g for 5 min. The cell pellet was then resuspended in 400 pl Hash-staining buffer (2% BSA and 0.01% Tween in PBS) and aliquoted, 45 pl per tube, into 8 tubes coated with Hash-staining buffer. To each sample aliquot, 5 pl Human TruStain FcX™ (BioLegend catalog #422302) was added, and incubated at 4° C. for 10 min for blocking. Then 1 pl CellHash antibody and 50 pl staining buffer (2% FBS in PBS) was added to each sample, which was distinct for each sample (TotalSeq™-A0257 to -A0265). The incubation was performed at 4° C. for 30 min. Next, 3 ml of Hash-staining buffer was added to each tube and the tubes were centrifuged at 4° C. for 5 min at 500 g. The supernatant was removed and the wash was repeated twice.

The hashed cells were processed with different conditions before GEM generation and barcoding. Specifically, the following conditions are tested. First, it was reasoned that mild lipid detergent during tagmentation can increase the accessibility of Tn5 enzyme into mitochondria. Accordingly, two groups were set, one with and the other without adding GDN in tagmentation. In each group, different fixation conditions were tested including 0.1% FA, 0.2% FA, 1% PFA, methanol, etc. High or low levels of BSA concentration were tested (The detailed condition is shown in FIG. 6). After tagmention, all cells were combined equally and loaded into one 10× genomics channel. The rest is following the standard protocol as mentioned above, except adding 0.2 uM Hash-Spikin primer. All three ReDeeM libraries were generated and sequenced.

The cell hashing data was resolved using in-house python script. The comparative analysis was performed for each modality across all testing conditions by controlling the same sequencing depth.

### Species Mix Experiment

With the modified protocol, there was concern about whether it can affect the leakage of mtDNA and RNA, because of the fixed and permeabilized cells. To examine that, a species mixing experiment was performed by 1:1 mixing a human K562 cell line and a mouse lung cancer cell. The mixed cells were processed with standard ReDeeM protocol and the leakiness and doublets rates were measured on all three modalities (FIG. 6).

### Dual Lineage Tracer Experiment

Mouse experiments were approved by the Massachusetts Institute of Technology Institutional Animal Care and Use Committee (IACUC) (institutional animal welfare assurance no. A-3125-01). A male mouse embryonic stem cell (mESC) line harbouring the conditional alleles KrasLSL-G12D/+ and Trp53fl/fl (KP) was engineered with the lineage tracer cassettes. The detailed engineering process, including vector information, tumour harvest and single-cell suspension were prepared as described in Yang et al.,⁴².

The single cells of 6 tumours were labelled with Cell Hash, and were profiled using ReDeeM except the following modification. Additional Target Site libraries were needed. The amplified cDNA libraries were further amplified with Target Site-specific primers containing Illumina-compatible adapters and sample indices (oDYT023-oDYT038, forward: 5′-CAAGCAGAAGACGGCATACGAGATNNNNNNNNGTCTCGTGGGCTCGGAGATGT GTATAAGAGACAGAATCCAGCTAGCTGTGCAGC (SEO ID NO: 1);
reverse: 5′-AATGATACGGCGACCACCGAGATCTACACNNNNNNNNTCTTTCCCTACACGACGCT CTTCC GATCT (SEQ ID NO: 2); “N” denotes sample indices) using Kapa HiFi ReadyMix (Roche), as described in Yang et al.,⁴².

For sequencing scRNA, scATAC, mtDNA libraries, the same strategy as described above for ReDeeM were used, except that four sets of mouse specific probes were designed to enrich mitochondrial fragments. For sequencing Target Site libraries, 15,000 total reads per cell were expected, and the following read lengths were used: Read1: 26 cycles, i7: 8 cycles, Read2: 290 cycles).

### Single-Cell Multi-Omics Data Preprocessing, Clustering, and Motif Analysis

The data preprocessing for the joint single-cell RNA and ATAC data was performed using 10× Genomics data preprocessing software Cell-ranger-arc. The basic quality control analysis was as follows: RNA UMI: 1,000~25,000 transcripts per cell, unique ATAC fragment: 1,000-70,000, Fragment on peak minimum percentage: 10%, minimum mtDNA copies per position per cell: 10×. Finally, the possible doublets are removed by Amulet (using default parameter). Unsupervised community detection-based clustering was performed on weighted nearest neighbour (WNN) using Seurat. Cell types are firstly annotated by gene expression using the latest signature (van Galen under review) which was further validated by sorted bulk RNA-seq data (found on the world wide web at sankaranlab.shinyapps.io/geneExpression/). The ATAC unique fragments for each clustered cell type were further aggregated to generate pseudo-bulk ATAC tracks. The single cell motif analysis was performed using ChromVar using the JASPAR human transcription factor (TF) database. The deviation of the TF frequency was computed for each cell for the downstream analysis.

Single-Cell eUMI-Based mtDNA Mutation Discovery

Unique molecule identifier (UMI) labels each original DNA molecule and has been widely used for rare variant calling to minimise artefacts that arise during PCR amplification and sequencing. The addition of UMI has been demonstrated to substantially improve the sensitivity and accuracy of mutation discovery (from 10⁻³ up to 10⁻⁷), and therefore it has been widely used in studying rare somatic mutations⁴⁰.

Neutral somatic mutation in mtDNA happens not very frequently (although more frequent than in the nuclear genome) but highly valuable as natural barcodes for lineage tracing. It was reasoned that the ability to accurately detect those low-frequency mtDNA mutations would be critical for lineage tracing technology that reconstructs fine-scale phylogenetic relationships.

The single-cell droplet based mtDNA fragments already had sufficient information to distinguish original single molecules, i.e., UMI could be used without introducing extra UMI barcodes. To be specific, the paired-end sequencing for each fragment provides information of single-cell barcode (CellBC) as well as Tn5 cutting sites on the left (start) and on the right (end). The CellBC-start-end is termed as endogenous UMI (eUMI). It was reasoned that the eUMI is sufficient to label unique molecules, because given a single cell (by CellBC), the Tn5 enzyme cutting sites that create fragments during transposition reaction are most likely unique across different mtDNA molecules in that given cell. The theoretic collision rate to use eUMI is in function of the mtDNA number per cell and the Tn5 cutting efficiency. The Tn5 cutting was simulated for 1000 times. The parameters used in these simulations were N (The number of mtDNA copies per cell), S (Size of mtDNA, 16.9 kb), and E (Tn5 cutting efficiency, or the dropout rate). N varies between 10 to 50,000 molecules per cell, while E is set as 0.1, based on real data. The result of the simulation showed that if the mtDNA is around or lower than a couple of thousand per cell, the eUMI is a robust method to label unique molecules with limited collision. And based on prior knowledge, the mtDNA copy number in hematopoietic cells is safely in that range⁷⁴.

The mutation calling pipeline is in following steps: 1, Data Preprocessing and eUMI grouping. 2, Consensus filtering. 3, Strand bias removal. 4, Lineage informative variants identification (Extend Data FIG. 21).

First, the nuclear mitochondrial DNA, or NUMT, regions are masked on the auto chromosomes as previously reported followed by mapping using bowtie2²⁹. Only the reads that were uniquely mapped to the mitochondrial genome (samtools view -bf 2 -q30) are used. Bases are filtered by sequencing quality with Q30 or higher. The bases that were sequenced in both read1 and read2 were processed separately from the bases with only one direction sequenced. After the preprocessing steps, the CellBC-start-end (eUMI) was extracted for each sequenced fragment and generated the eUMI groups for the consensus variant calling. The following consensus filtering parameters were applied. The minimum eUMI group size was 2 or 3, depending on whether the given base was read by one direction or both directions. The consensus score must be 100% if eUMI group size was smaller than 4, whereas the consensus score threshold was 75% if the eUMI group size was equal or larger than 4. After the consensus filtering, a binomial modeling method is used to evaluate and remove the significantly strand-biased mutations (p value <0.01, fold>2). The NUMT is a potential confounder for the real mtDNA mutations. Previously in bulk studies, heterozygous NUMT mutation was modeled to control this contamination⁷⁵. Here, following the same principle, the mutation allele numbers per cell is examined. If a mutation appeared to have 2 or more than 2 alleles in one single cell, that mutation was considered as mtDNA mutations. Otherwise, the mutation would be removed. Finally, homoplasmic mutations that appeared in all single cells are removed as are all the heteroplasmic mutations that were only detected in one single cell, both of which were not lineage informative.

mtDNA Mutation Burden Analysis

The mtDNA mutation burden is estimated using a quantitative method. The number of detected mutations per cell is in function of the biological mutation burden as well as the technical detectability that is influenced by the mtDNA capture rate. The mtDNA mutation burden is calculated by normalising against the mtDNA coverage (#mtDNA copies per position per cell) as well as the eUMI filtering rate which was used to correct technical batch effects across different experiments due to different sequencing depth, sequencing quality, etc. Given a single cell i in Sample j, the mutation burden is computed as below

$$Mutation burden = \frac{\# mtDNA mutations \vert Cell i}{( eUMI filtering rate \vert Sample j ) \times ( mtDNA coverage \vert Cell i )}$$

Infer Cell-to-Cell Lineage Distance and Phylogenetic Tree Using mtDNA Mutations

After all the filtering steps (FIG. 7), a sparse matrix C was generated to contain all the variant allele count (cell versus mtDNA mutation). The allele count matrix was further divided by the matrix of mtDNA copy number per position per cell, which generated a heteroplasmy matrix H for visualisations. Because mutation count data was sparse, the quantitative heteroplasmy level was susceptible to the variation of mtDNA coverage. To minimise the coverage bias in the downstream analysis, binarization of the matrix C into matrix C_{bin} was performed.

Based on matrix Chin, the cell-to-cell weighted Jaccard distance was computed. The prior of the mtDNA mutation frequency across multiple donors was used to weight the Jaccard distance to account for the potential homoplasy. Intuitively, the weighted Jaccard distance measures the level to which any two cells do not share mutations, i.e., After proper normalisation, the more mtDNA mutations are shared, the closer the two cells' relationships are. First a prior probability for each mutation was defined which prioritises mutations with lower mutation rate across donors. (i.e. less likely to be the same mutation occurring independently). For cell x and cell y, the weighted jaccard distance is defined below

Priorᵢ=(1−Average mutation rate across donors)

$${D}_{w \_ jaccard} = 1 - \frac{\sum i ε [ {x}_{i} = 1 \& {y}_{i} = 1 ] {Prior}_{i}}{\sum i {Prior}_{i}}$$

Next, the weighted Jaccard distance was feed into the neighbour joining algorithm for the phylogenetic tree reconstruction and visualisations using ape, ggtree packages.

Orthogonal Validation of the mtDNA-Based Lineage Tracing Using Dual-Tracer Dual

The dual tracer experiment generated single-cell data that simultaneously contained 5 modalities (Cell Hashing, scRNA, scATAC, mtDNA, Target Site) in the same cell. The scRNA and scATAC modalities were used to identify cells that passed the quality control. The Cell Hashing data were used to split the data into 6 tumours using an in-house python script. The Target Site data were indels randomly created by Crispr-based lineage tracer and will be used to compare with the mtDNA mutation-based lineage inferences.

The preprocessing of mtDNA library data followed the standard ReDeeM pipeline as described above. The Target Site library data was preprocessed using Python package Cassiopeia⁷⁶. Briefly, reads with identical cellBC and UMI were collapsed into a single, error-corrected consensus sequence representing a single-expressed transcript. Consensus sequences were identified within a cell based on a maximum of 10 high-quality mismatches (PHRED score greater than 30) and an edit distance less than 2 (default pipeline parameters). UMIs within a cell reporting more than one consensus sequence were resolved by selecting the consensus sequence with more reads. Each consensus sequence was aligned to the wild-type reference Target Site sequence using a local alignment strategy, and the intBC and indel alleles were called from the alignment. Cells with fewer than 2 reads per UMI on average or fewer than 10 UMIs overall were filtered out. These data were summarised in a molecule table which records the cellBC, UMI, intBC, indel allele, read depth, and other relevant information. Then, character matrices were formed summarising indel information across the N cells in a population and their M cut-sites. Characters (i.e., cut-sites) with more than 80% missing information or containing a mutation that was reported in greater than 98% of cells were filtered out for downstream analysis.

The character matrices were then used to compute a hamming distance (hamming_distance in Cassiopeia) that describes the cellular lineage relationships. The hamming distance was then dimension reduced using multidimensional scaling (MDS) using function cmdscale in R for visualisation. The mtDNA mutations were overlaid on the MDS map.

To systematically benchmark the cellular lineage distance between mtDNA mutation-based and the CRISPR indel-based recorder, two independent k nearest neighbourhoods were generated based on mtDNA mutation-derived Jaccard distance (Graph G^{mt}) and CRISPR indel-derived hamming distance (Graph G^{cr}). a metric “Agreement of closeness” was defined to quantify the degree to which the mtDNA mutation-based lineage relationships were supported by CRISPR tracer. Given any single cell X, k (default is 20), nearest neighbours M1, M2 was identified. Mk in mtDNA mutation derived graph G^{mt}. Then the average distance from M1, M . . . Mk to the cell X on G^{cr} was computed. The ranks of these distances on G^{cr} (The closest is indicated as rank #1) were compared with that from randomly picked k cells. The random process was repeated 1000 times and the rank % closer toward cell X in observed data than expected was defined as “agreement of closeness”. (i.e. Positive agreement of closeness indicates a closer distance to cell X than expected, while negative score indicates farther). The empirical p value was also generated by I permutation (default is 1000). For any given cell X.

$${Rank}_{observed} = rank ( \frac{1}{20} {\sum }_{k = 1}^{20} distance ( {M}_{k} ∈ {G}^{cr} , X ∈ {G}^{cr} ) )$$

$${Rank}_{i} = rank ( \frac{1}{20} {\sum }_{k = 1}^{20} distance ( random cell k ∈ {G}^{cr} , X ∈ {G}^{cr} ) )$$

$$Agreement of closeness = \frac{1}{100} {\sum }_{i = 1}^{1000} \frac{( {Rank}_{i} - {Rank}_{observed} )}{Total cell number}$$

### Cell Type Subclonal Origin Analysis

First, “lineage-informative” mtDNA mutations was selected by modelling the distributions across all cell types. The mutations that are randomly distributed which likely arose in certain unbiased stem cell clones and thus were less informative to study the cell type subclonal origins were removed. Using the “lineage-informative” mtDNA mutations, the matrix Cbin was generated and computed the weighted Jaccard distance. Then a k-nearest neighbour (KNN) was generated. Graph G that describes the cell-to-cell lineage relationships based on the shared mutations. Then the cell type annotations were integrated from the multiomics analysis onto the graph G. For any given cell (query cell), the proportion of each cell type (target cell types) within the k nearest neighbors on graph G was computed. The target cell type proportions for each query cell type were then aggregated and scaled. Finally, the query cell types were grouped by hierarchical clustering based on the distributions of target cell type proportions.

### HSC Multi-Omics Subpopulation and Clonal Preference Analysis

To specifically study human hematopoietic stem cells (HSCs), the CD34⁺CD45RA⁻ CD90⁺ population was experimentally enriched as described previously. The HSC populations were further refined using a semi-unsupervised method. First, community detection-based clustering was performed for all the cells on weighted nearest neighbour (WNN) using Seurat. Second, the HLF gene expression level was averaged for each cluster and defined HLF^{hi} and HLF^{low} clusters. Third, the HLF and CRHBP gene expression levels was simultaneously examined for every single cell. Any HSC cell is required to highly express both HLF and CRHBP and was also grouped within HLF^{hi} clusters. The defined HSCs cells were further examined using orthogonal HSC signatures, including MECOM, HOPX, AVP, MLLT3, etc⁵⁴. To improve the robustness of lowly expressed genes, the expression data were enhanced using Rmagic package for visualizations⁷⁷.

For the refined HSCs above, a secondary clustering on WNN was performed to define subpopulations. The subpopulations were identified using Seurat with resolution 0.6. The subpopulations were visualised on RNA-based, ATAC-based and WNN-based UMAP. The differentially expressed genes and accessible chromatins were identified using FindMarker function by Seurat. The DNA binding motifs were analysed by FIMO scanning with the JASPAR human TF motif database followed by a binomial test across HSC subpopulation-specific open chromatin peaks. The visualisation of differential motifs were performed by chromVar.

To best capture the principal HSC clonal structures, normalisation and dimension reduction were performed using TF-IDF (term frequency inverse document frequency) and SVD (singular value decomposition) on a binarized variant-by-cell matrix. The top 30 LSI (latent semantic indexing) were used to measure Euclidean distance that was further passed on to the neighbour joining algorithm to build the phylogenetic tree. Next, the mtDNA mutations were assigned to the tree branches using a maximum likelihood method as described previously, which has been incorporated in redeemR (Add_AssignVariant function)²⁷. “HSC clonal group” was defined as the minimum clade unit that at least contains 50 single cells and have one or more than one confidently assigned mutations (p>0.6).

Next, the distribution of each HSC clonal group was examined across all the HSC subpopulations that were defined by RNA- and ATAC-based cell state. Compared to the background, the fold change of a given clonal group proportion in a given cell-state subpopulation was computed and the p value was estimated by binomial test. The fold changes and p values were compared across HSCs from two sampling time points.

### HSC-Progeny Assignment Based on Network Propagation

With the sampling of both the HSCs as well as the committed and differentiated progenies in the same donor, the mtDNA mutation profiles were used to assign the progies into each HSC clonal group. Because the variant-by-cell matrix is highly sparse, the task of confident single-cell assignment is challenging. Previous studies showed that phenotypic relevance of individual cells can be faithfully modelled in a cell-to-cell similarity graph and effectively identified by network propagation algorithm⁶¹, despite the inherent high dimensionality and extensive sparsity nature of single cell genomics data. Therefore, a new method, SCAVENGE-L, (Single Cell Analysis of Variant Enrichment through Network propagation of GEnomic data for lineage tracing data) was developed using the network propagation strategy that takes advantage of neighbourhood information and efficiently assigns the cells with probabilistic metrics. This network propagation strategy effectively overcomes the data sparsity in single cell epigenomic data analysis⁶¹. The clonal structure of individual cells could also be faithfully distilled into a network, where each node represents a cell and each edge represents mutation profile similarity among cells. By defining cells of interest (i.e., a group of stem cells), this network can be exploited to search highly relevant cells (i.e., progeny) using both network topology structure and cell-to-cell distance.

First a full binarized variants-by-cell matrix was generated that included all stem, progenitor and differentiated cells from a given donor. TF-IDF was performed followed by SVD for normalisation and dimension reduction. The top 30 LSIs were used for constructing a mutual K nearest neighbour graph (mKNN). Next, each HSC clonal group was highlighted on the mKNN graph. A random walk with restart method was used for discovering the progeny for cells of each HSC clonal group that were termed as seed cells. The information on this mKNN graph can spread across, and the information retained in the network at stationary state can be used to measure the probability of any given cell belonging to an HSC clonal group (seed cells). The network propagation analysis was performed with damping factor 0.05 from each HSC clonal group (seed) iteratively. Finally, this generated a cell-by-clonal group probability matrix that measured the confidence of assignment. The max probability >0.7 was used as the cutoff to filter out ambiguous progenies.

### Clonal Output and Lineage Bias Analysis

To study the HSC clonal output activities, both the HSCs and all differentiated progenies were collected from the same donor across two sampling time points. Based on the mtDNA mutations, SCAVENGE-L was applied to assign differentiated progenies to each HSC clone. Next, the clonal output level was measured by counting the number of progenies for each HSC clonal group followed by normalisation with HSC clone size (the number of HSCs per clonal group). The clonal output level was compared across the two sampling time point and computed Pearson's correlation. To evaluate the contribution of the hematopoiesis across different HSC clones, the clones were ranked from highest to lowest and computed the cumulative proportion of the differentiated progenies contributed by these HSC clones.

Next, for each HSC clone, the proportion of 4 main lineages was computed: Myeloid (monocytes, GMP, MDP, cDC), Erythroid (MEP, EryP), Meg (MKP) and Lymphoid (CD4, CD8, NK, B, ProB, CLP). The lineage biases were modelled by binomial distribution against the background by all cells. The enrichment fold change was calculated for each sampling time point independently. Finally, the HSC clonal output levels and lineage biases were scaled and Pearson's correlations were computed.

### Clonal Expansion Analysis in Ageing

First, both BMMC and CD34⁺ HSPC were collected for two young donors (31 year old female and 26 year old male, or young-1 and young-2) as well as two aged donors (76 year old male and 78 year old male, or aged-1 and aged-2). Using the same consensus variant calling pipeline and neighbour joining algorithm described previously, the phylogenetic tree for all 4 donors was reconstructed. The clonal expansions were estimated in two methods: clone-based and clade bases. For the clone-based method, first the clonal groups were identified as described above. Briefly, the variants were assigned to branches probabilistically, the tree was cut down on branches that have at least n confident variants, and with clone group size at least m. The parameters involved were m (minimum number of cells as a clone, default is 50), n(minimum number of cumulative variants on the branch to cut, default is 1), p (The probability of the variant to be assigned, default is 0.6), and D (Dump small clones with less than D cells). The distribution of clone sizes was compared between young and aged donors by cumulative proportions. To rule out the potential bias of the parameters that define clone groups, the parameter combinations were adjusted (m,n,p,D) and compared the clonal size distribution between young and aged donors (FIG. 14c). Next, the Shannon diversity index S was also computed for each donor to measure the clonal diversity between young and aged. Given a clone group i, the Sizei is the cell number of that clone. The Shannon diversity index S is calculated as below

$$N = {\sum }_{1}^{i} {Size}_{i}$$

$$S = {\sum }_{1}^{i} \frac{{Size}_{i}}{N} \times log ( \frac{{Size}_{i}}{N} )$$

For the clade-based method, the expansion clades were identified as previously described and implemented using the function “cassiopeia.tl.corpute_expansion_pvalues” from Cassiopeia package (publicly available on the world wide web at github.com/YosefLab/Cassiopeia)⁷⁶. Briefly, the number of cells contained in the subclone were compared to its direct “sisters” and computed a probability of this observation under neutral selection with a coalescent model. The clades with p-value less than 0.01 and with at least 5% cells were annotated as expanded clades. Finally, the proportions of cells contributed by the expanded clades were summarised for each donor.

### Infer Single Cell Fitness

The phylogenetic structure can be used to infer the cell fitness⁶⁵. The function “infer_fitness” was applied from the jungle package (publicly available on the world wide web at github.com/felixhorns/jungle) which implements a previously described probabilistic method for inferring relative fitness coefficients between samples in a clonal population⁶⁵.

### Loss of Chromosome Y Analysis

The loss of chromosome Y was inferred at single cell level using the scATAC reads. From uniquely mapped reads by CellRanger-arc (bam file), the PCR duplicates were firstly removed and counted the number of unique fragments per chromosome per cell. The number of chrY fragments per cell were modelled using binomial distribution out of the total fragment numbers. The cells of LOY were defined for the ones with chrY fragment counts 10 fold lower than expected (p-value<0.001). The local LOY density (dLOY) was computed as dLOY=number of cells of LOY/Clade size. The enrichment score was computed as the Z-score of dLOY normalised by total density. The enrichment was further tested using a one-tailed binomial test.

### Data Availability

ReDeeM is further computationally empowered by consensus variant calling pipeline REDEEM-V (found on the world wide web at github.com/chenweng1991/redeemV), and an inhouse R package REDEEM-R (found on the world wide web at github.com/chenweng1991/redeemR) for the downstream mutation quality control, as well as single cell phylogenetic and integrative analysis.

### Example 2: ReDeeM Protocol

### Introduction

Protocol for single-cell Regulatory multi-omics with Deep Mitochondrial mutation profiling. Following this protocol, three separate libraries plus one optional library (mtDNA lib; RNA lib, ATAC lib, and an optional Hashing library) will be generated in parallel for joint single-cell profiling. This protocol has been tested for hematopoietic stem/progenitor and differentiated cells (Including T cells, B cells, NK cells, monocyte, dendritic cells, plasma, GMP, MDP, CLP, MPP, HSC, etc). For other tissues or cell types, some conditions may need to be further optimized.

### Materials

(Optional) TotalSeq™-A0301 anti-mouse Hashtag Antibody (A0301-A0310)
(Optional) TruStain FcX™ (BioLegend, #422302)
Pierce™ 16% Formaldehyde (w/v), Methanol-free (Thermo Scientific, #28906)
BSA (Sigma, A4503-100G)
Nonidet-P40 substitute (USbiological CAS: 9036-19-5)
10× Genomics multiome kit (10× Genomics, PN-1000283/PN-1000285)
glyco-diosgenin, GDN (Avanti Polar Lipids, 850525P-500MG)
NEBNext HF PCR MM (NEB, #M0541L)
Protector RNase Inhibitor (Sigma, #3335402001)
xGen™ Hybridization and Wash v2 Reagents, 16 rxn (IDT, #10010351)
xGen™ Hybridization and Wash v2 Beads, 16 rxn (IDT, #10010353)
xGen™ Universal Blockers NXT (IDT, #1079584)
KAPA HiFi HotStart ReadyMix (Kapa Biosystems, KK2601)
SYBR Green I Nucleic Acid Gel Stain—10,000× concentrate in DMSO (Life Technologies #S7563)
Agilent High Sensitivity DNA Kit (Agilent #5067-4626)
In-house buffers
   Fixation buffer: 0.1% Formaldehyde. 0.04% BSA, 0.2 U/ul RNase inhibitor in DPBS
   FACS buffer: 2% BSA in DPBS
   DPBS/RI buffer: 0.04% BSA, 0.2 U/ul RNase Inhibitor in DPBS
   Perm buffer: 10 mM Tris-HCl, 10 mM NaCl, 3 mM MgCl₂, 0.1% NP40, 1 mM DTT, 1 U/ul Rnase inhibitor in ddH2O
   Perm-Wash buffer: 10 mM Tris-HCl, 10 mM NaCl, 3 mM MgCl₂, 0.02% BSA, 1 mM DTT, 0.2 U/ul RNase inhibitor in ddH2O
   Resuspension buffer: 1 mM DTT and 1 U/ul Rnase inhibitor in 1× nuclei buffer from multiome kit (10× Genomics, PN-1000283/PN-1000285)
   ATAC-GDN buffer: 1% GDN in 1×ATAC buffer (PN-2000193)
Oligo sequences

(SEQ ID NO: 3)
(Optional) HTO additive primer:
5′-GTGACTGGAGTTCAGACGTGTGCTCTTCCGAT*C*T-3′
(Optional) D701_LONG primer:
(SEQ ID NO: 4)
5′-CAAGCAGAAGACGGCATACGAGATCGAGTAATGTGACTGGAGTTCAG
ACGTGTGCTCTTCCGAT*C*T-3′
(Optional) Hash-primer:
(SEQ ID NO: 5)
5'-AATGATACGGCGACCACCGAGATCTACACTCTTTCCCTACACGACG
C*T*C-3′
Nextera oligos: N701-N712:
(SEQ ID NO: 6)
5′-CAAGCAGAAGACGGCATACGAGAT[NNNNNNNN]GTCTCGTGG
GCTCGG-3′
P5:
(SEQ ID NO: 7)
5′-AATGATACGGCGACCACCGAGA-3′
P7:
(SEQ ID NO: 8)
5′-CAAGCAGAAGACGGCATACGAGAT-3′

### Procedure

### (Optional) Cell Hashing for Multiple Samples

1. Centrifuge 100K~1M Bone Marrow Mononuclear Cells (BMMCs) for each sample at 500 g for 5 min.
2. Resuspend in 50 μl FACS buffer.
3. Add 5 μl Human TruStain FcX™ in each sample, mix and incubate at 4° C. for 10 minutes to for blocking.
4. During the incubation, for each sample, dilute 1 μl TotalSeq™ antibody (unique for each sample) in 50 μl FACS buffer in a corresponding fresh, 1.5 ml, low-binding tube
5. Transfer the corresponding 50 ul antibody dilution from step 4 into each of the blocked-cell suspensions, and incubate at 4° C. for 30 minutes.
6. Add 3 ml FACS buffer in each sample and centrifuge 500 g at 4° C. for 5 minutes for washing.
7. Remove supernatant and repeat the wash 1 more time with 3 ml FACS buffer for each sample and centrifuge 500 g 4° C. for 5 min
8. Pool Hashed samples together (usually equal cell number) as one sample to proceed forward.

### Fixation and Permeabilization

Note: Decreasing BSA concentration in most of the buffers can improve the mtDNA recovery rate. The in-house buffers provided in Material section are optimized for hematopoietic cells. The recipes can be further optimized for different tissues and cell types.
9. For each sample, resuspend cells in 1 ml Fixation buffer, and incubate at room temperature for 10 minutes.
10. Quench the fixation reaction with 50 ul glycine (2.5M) at final concentration of 0.125M, and incubate at room temperature for 3 minutes.
11. Centrifuge 500 g at 4° C. for 5 minutes.
12. Remove supernatant, add 3 ml cold PBS/RI, and centrifuge 500 g at 4° C. for 5 minutes.
13. Remove supernatant, add 1 ml Perm buffer, and incubate for 5 minutes on ice. Then immediately add Perm-Wash buffer 3 ml, and centrifuge 500 g at 4° C. for 5 minutes.
14. Remove supernatant, add 200 ul Resuspension buffer, and centrifuge 500 g at 4° C. for 5 minutes.
15. Remove the top of supernatant without disturbing cell pellet, leaving 20-100 ul Resuspension buffer, and resuspend and count the cells. Ideally, make the cell concentration equal to or more than 2800 cell/ul
   Tagmentation with GDN
   Note: Adding GDN during tagmentation can increase the mtDNA recovery rate without significantly affecting RNA and ATAC quality.
16. Generate the following mix per sample for tagmentation:

TABLE 3
Tagmentation Mix

| Name | Volume per reaction (ul) |
|---|---|
| ATAC buffer B (10x Genomics, | 5.5 |
| PN-2000193) | |
| ATAC-GDN buffer (In-house buffer) | 1.5 |
| ATAC Enzyme B (10x Genomics, | 3 |
| PN-2000265/2000272) | |
| Cells at a concentration of | 5 |
| 2800 cells/μl (Step 15) | |

17. Incubate each sample at 37° C. for 1 hour, and immediately proceed to next step.

### GEM Generation & Barcoding

18. Use the tagmented cells from step 17 to proceed, following 10× Genomics multiome user guide revB CG000338-Step2 for GEM generation and barcoding

### Post GEM Incubation Cleanup

19. Following 10× Genomics multiome user guide revB CG000338-Step3 for Post GEM Incubation Cleanup.

### Pre-Amplification PCR

20. Follow 10× Genomics multiome user guide revB CG000338-Step4, except
   a) (Optional, if cell hashing was performed) Spike in 1 ul 0.2 uM HTO additive primer at 10× Genomics multiome user guide revB CG000338-Step4.1
   b) Elute in 100 ul Buffer EB (CG000338-Step4.3) to generate Pre-Amp library

### ATAC Library Prep (Small-Scale Pilot Test)

21. Use 1 μL of Pre-Amp library in pilot test for ATAC PCR with monitoring using SYBR Green

TABLE 4
ATAC qPCR Mix

| Component | Volume per reaction (ul) |
|---|---|
| Pre-Amp (Step 20) | 1 |
| NEBnext mix | 12.5 |
| N701 primer | 1.2 |
| SI PCR primer B(10X Genomics, PN-2000128) | 1.2 |
| 100X SYBR green | 0.25 |
| ddH2O | 8.85 |
| Total | 25 |

Run qPCR using the following program: 1. 98° C. 45 s; 2. 98° C. 10 s; 3. 63° C. 30 s; 4. 72° C. 20 s; 5. Read Plate; 6. Go to step 2 for 35 more cycles. Stop the qPCR manually based on the amplification curve. Usually around 10 or below 10 cycles when the curve is reaching plateau.
22. Run Bioanalyzer for the qPCR product using Agilent High Sensitivity DNA Kit.

### ATAC Library Prep (Scale-Up)

23. Scale up the PCR above to 8 wells per sample, (50 μL per well, for a total 400 μL PCR mix), monitored using SYBR Green. Each well should contain:

TABLE 5
PCR Set up

| Component | Volume per reaction (ul) |
|---|---|
| Pre-Amp | 5 |
| NEBnext mix | 25 |
| N701 | 2.4 |
| SI PCR primer B(10X Genomics, PN-2000128) | 2.4 |
| 100X SYBR Green | 0.5 |
| ddH2O | 14.7 |
| Total | 50 |

Run qPCR using the same program in small-scale test: 1. 98° C. 45 s; 2. 98° C. 10 s; 3. 63° C. 30 s; 4. 72° C. 20 s; 5. Read Plate; 6. Go to step 2 for 35 more cycles. Stop the qPCR manually based on the amplification curve. Limit the number of cycles to 10 or lower.
24. Purify the PCR product.
   Combine each 2 wells (into 100 ul) for purification. Purify by SPRI beads with size selection 0.4×-1.55×. Elute in 20 ul*4 ddH2O (80 ul total), The expected DNA yield is more than 3000 ng.
25. Run Bioanalyzer for the qPCR product using Agilent High Sensitivity DNA Kit.
26. Ready for sequencing. Paired-end illumina sequencing recommended. R1: 150 nt, i7: 8 nt, i5: 24 nt, R2:150 nt. 20K-25K sequencing reads are suggested.
27. The product from step 25 is also ready for mitochondrial DNA capture. Typically, 2000 ng is needed.
   Mitochondrial DNA (mtDNA) Library Prep
28. For each sample, prepare 500 ng ATAC library ×4 (2000 ng in total) for four hybridization reactions using staggered probes (mitoV1, mitoV2, mitoV3, mitoV4)
29. The hybridization is performed by following IDT xGen™ hybridization capture guide. Briefly, the capture is a two-day protocol as shown below.
30. D1: Setting up hybridization reaction following APPENDIX A in xGen™ hybridization capture guide. xGen Hyb Panel is replaced by one of the probes for each reaction: mitoV1/mitoV2/mitoV3/mitoV4. The hybridization reaction is as below:

TABLE 6
Hybridization reaction Set up

| Component | Volume per reaction (ul) |
|---|---|
| xGen 2X Hybridization Buffer | 9.5 |
| xGen Hybridization Buffer Enhancer | 3 |
| xGen Universal Blocker NXT | 2 |
| Probe mitoV1/mitoV2/mitoV3/mitoV4 | 4.5 |
| Total | 19 |

The hybridization reaction is performed 95° C. 30 s, followed by 65° C. 16 h
31. D2: Capture and washes are performed following Tube protocol xGen™ hybridization capture guide (from Page 23).
32. The post-capture PCR is monitored by SYBR: 1 well of PCR (as below) is performed for each hybridization reaction.

TABLE 7
PCR hybridization set up

| Component | Volume per reaction (ul) |
|---|---|
| Beads after washes | 19.7 |
| KAPA ReadyMix | 25 |
| P5 primer | 2.4 |
| P7 primer | 2.4 |
| SYBR | 0.5 |
| Total | 50 |

Run qPCR using the following program: 1. 98° C. 45 s; 2. 98° C. 10 s; 3. 63° C. 30 s; 4. 72° C. 20 s; 5. Read Plate; 6. Go to step 2 for 35 more cycles. Stop the qPCR manually based on the amplification curve. Limit the number of cycles to 3-5 cycles.
33. Combine every four wells for each sample (corresponding to 4 hybridizations using probes mitoV1, mitoV2, mitoV3, mitoV4) in total 200 ul and purify by 1.6×SPRI beads (320 ul SPRI each), finally elute in 25 ul ddH2O.
34. Run Bioanalyzer for the purified PCR product using Agilent High Sensitivity DNA Kit.
35. Ready for sequencing. For sensitive variant calling, paired-end illumina sequencing is recommended. RI: 150 nt, 17: 8 nt, i5: 24 nt, R2:150 nt. Deep sequencing with 50K-60K sequencing reads per cell for mtDNA library is recommended.

### RNA Library

36. RNA library is prepared following 10× Genomics multiome user guide revB CG000338-Step6 and Step7.
37. Ready for sequencing. Paired-end illumina sequencing following 10× Genomics guide is recommended: RE: 28 nt, i7: 10 nt, i5: 10 nt, R2:90 nt.

### (Optional) Hashing Library

38. If cell hashing was performed, the hashing library is prepared using PCR as below, which is also monitored by SYBR

TABLE 8
Hashing library PCT set up

| Component | Volume per reaction (ul) |
|---|---|
| Pre-Amp | 5 |
| NEBnext mix | 12.5 |
| D701_LONG | 1.2 |
| Hash-primer | 1.2 |
| 100X SYBR Green | 0.25 |
| ddH2O | 4.85 |
| Total | 25 |

Run hashing qPCR using the following program: 1. 98° C. 45 s; 2. 98° C. 10 s; 3. 63° C. 30 s; 4. 72° C. 20 s; 5. Read Plate; 6. Go to step 2 for 35 more cycles. Stop the qPCR manually based on the amplification curve. Usually 15-20 cycles are needed.

1-43. (canceled)

44. A method of determining a cell lineage or a cell state of a single cell in a multicellular eukaryotic organism, comprising:
detecting mutations in mitochondrial genome consensus sequencing reads of single cells obtained from a subject to generate mitochondrial genome mutation data, the mitochondrial genome consensus sequencing reads having been obtained by single-cell targeted sequencing of the mitochondrial genome and unique molecular identifier (UMI) sequencing error correction; and
assigning a lineage and/or status group to the single cells based on at least the mutations in the mitochondrial genome mutation data.

45. A method of determining a cell lineage or a cell state of a single cell in a multicellular eukaryotic organism, comprising:
detecting mutations in mitochondrial genome consensus sequencing reads of single cells obtained from a subject to generate mitochondrial genome mutation data;
measuring gene expression levels for the single cells to generate gene expression level data; and
detecting chromatin accessibility for the single cells to generate chromatin accessibility data;
wherein the mitochondrial genome mutation data, the gene expression level data, and the chromatin accessibility data of a particular single cell are associated with each other; and
assigning a lineage and/or status group to the single cells based on the mutations in the mitochondrial genome mutation data, the gene expression level data, and the chromatin accessibility data.

46. The method of claim 45, further comprising generating a phylogenetic tree for the single cells based on the mutations in the mitochondrial genome mutation data, the gene expression level data, and the chromatin accessibility data of the single cells.

47. The method of claim 46, further comprising displaying the phylogenetic tree.

48. The method of claim 45, wherein detecting chromatin accessibility in the single cells comprises single cell ATAC-seq (scATAC-seq) analysis.

49. The method of claim 45, wherein measuring gene expression of the single cells comprises single cell RNA sequencing (scRNA-seq) analysis.

50. The method of claim 45, wherein the single-cell targeted sequencing of the mitochondrial genome comprises hybrid capture of targeted sequences using one or more sets of probes.

51. The method of claim 45, wherein detecting mutations in mitochondrial genome consensus sequencing reads comprises calling of mitochondrial genome mutations, comprising:
a) detecting a mutation in a sequence that uniquely maps to the mitochondrial genome; wherein
b) the mutation base is detected in reads of both strands;
c) the mutation base is in a read that is greater than 30 nucleotides;
d) the endogenous unique molecular identifier (eUMI) for each sequenced fragment is extracted and the percentage of mutant reads in a UMI group (consensus score) is greater than 75; and
e) the UMI group size is 2 or more.

52. The method of claim 45, wherein the mitochondrial genome consensus sequencing reads are obtained from a scATAC-seq library.

53. The method of claim 45, comprising sequencing the mitochondrial genome of the single cells to generate mitochondrial genome sequencing reads, and aligning the mitochondrial genome sequencing reads to obtain mitochondrial genome consensus sequencing reads.

54. The method of claim 53, comprising fixing and permeabilizing the single cells prior to the sequencing, wherein the single cells are fixed in a fixation buffer comprising an aldehyde, albumin, and/or an RNase inhibitor; and wherein the single cells are permeabilized in a permeabilization buffer comprising a surfactant.

55. The method of claim 54, wherein the fixation buffer comprises 0.01%-1% formaldehyde, 0.001%-2% bovine serum albumin (BSA) and 0.1-1 U/ul RNase Inhibitor (RI); and
wherein the permeabilization buffer comprises 10 mM Tris, 10 mM NaCl, 1-5 mM MgCl₂, 0.01-1% NP40, mM DTT, 0%-2% BSA and 0.1-2 U/ul RI.

56. The method of claim 55, wherein the fixation buffer comprises 0.1% formaldehyde, 0.01% bovine serum albumin (BSA) and 0.2 U/ul RNase Inhibitor (RI); and
wherein the permeabilization buffer comprises 10 mM Tris, 10 mM NaCl, 3 mM MgCl₂, 0.01% NP40, 0.5-2 mM DTT, 0.01% BSA and 1 U/ul RI.

57. A method of fixing and permeabilizing whole cells, comprising:
(a) fixing the cells by contacting the cells with a fixation buffer comprising an aldehyde, an RNase inhibitor (RI), and albumin; and
(b) permeabilizing the cells, after the fixing, by contacting the cells with a permeabilization buffer comprising a surfactant, MgCl₂, DTT, albumin, and RI;
wherein the albumin is bovine serum albumin (BSA),
wherein the aldehyde is formaldehyde,
wherein the surfactant is NP-40.

58. The method of claim 57, wherein the fixation buffer comprises 0.01%-1% formaldehyde, 0.001%-2% bovine serum albumin (BSA) and 0.1-1 U/ul RNase Inhibitor (RI); and
wherein the permeabilization buffer comprises 10 mM Tris, 10 mM NaCl, 1-5 mM MgCl₂, 0.01-1% NP40, mM DTT, 0%-2% BSA and 0.1-2 U/ul RI.

59. The method of claim 57, wherein the fixation buffer comprises 0.1% formaldehyde, 0.01% bovine serum albumin (BSA) and 0.2 U/ul RNase Inhibitor (RI); and
wherein the permeabilization buffer comprises 10 mM Tris, 10 mM NaCl, 3 mM MgCl₂, 0.01% NP40, 0.5-2 mM DTT, 0.01% BSA and 1 U/ul RI.

60. The method of claim 59, wherein the permeabilization buffer does not comprise BSA.

61. The method of claim 57, wherein the cells are in a tissue preparation or tissue section.

62. The method of claim 57, wherein the fixing of whole cells takes place at room temperature for 5-20 minutes, and the permeabilizing of whole cells takes place on ice for 1-10 minutes.

63. The method of claim 57, further comprising sequencing at least a portion of the mitochondrial genomes of single cells, detecting mutations in mitochondrial genome consensus sequencing reads of single cells obtained from a subject to generate mitochondrial genome mutation data;
measuring gene expression levels for the single cells to generate gene expression level data; and
detecting chromatin accessibility for the single cells to generate chromatin accessibility data;
wherein the mitochondrial genome mutation data, the gene expression level data, and the chromatin accessibility data of a particular single cell are associated with each other; and
assigning a lineage and/or status group to the single cells based on the mutations in the mitochondrial genome mutation data, the gene expression level data, and the chromatin accessibility data.


SEQUENCE LISTING (28 sequences)

SEQ ID NO: 1 - DNA, 86 bp, synthetic construct
  source 1..86: mol_type = other DNA
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
caagcagaagacggcatacgagatnnnnnnnngtctcgtgggctcggagatgtgtataagagacagaatccagctagctgtgcagc

SEQ ID NO: 2 - DNA, 66 bp, synthetic construct
  source 1..66: mol_type = other DNA
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
aatgatacggcgaccaccgagatctacacnnnnnnnntctttccctacacgacgctcttccgatct

SEQ ID NO: 3 - DNA, 34 bp, synthetic construct
  source 1..34: mol_type = other DNA
  modified_base 32: mod_base = OTHER; note = Nucleotide with phosphorothioate linkage
  modified_base 33: mod_base = OTHER; note = Nucleotide with phosphorothioate linkage
gtgactggagttcagacgtgtgctcttccgatct

SEQ ID NO: 4 - DNA, 66 bp, synthetic construct
  source 1..66: mol_type = other DNA
  modified_base 64: mod_base = OTHER; note = Nucleotide with phosphorothioate linkage
  modified_base 65: mod_base = OTHER; note = Nucleotide with phosphorothioate linkage
caagcagaagacggcatacgagatcgagtaatgtgactggagttcagacgtgtgctcttccgatct

SEQ ID NO: 5 - DNA, 49 bp, synthetic construct
  source 1..49: mol_type = other DNA
  modified_base 47: mod_base = OTHER; note = Nucleotide with phosphorothioate linkage
  modified_base 48: mod_base = OTHER; note = Nucleotide with phosphorothioate linkage
aatgatacggcgaccaccgagatctacactctttccctacacgacgctc

SEQ ID NO: 6 - DNA, 47 bp, synthetic construct
  source 1..47: mol_type = other DNA
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
caagcagaagacggcatacgagatnnnnnnnngtctcgtgggctcgg

SEQ ID NO: 7 - DNA, 22 bp, synthetic construct
  source 1..22: mol_type = other DNA
aatgatacggcgaccaccgaga

SEQ ID NO: 8 - DNA, 24 bp, synthetic construct
  source 1..24: mol_type = other DNA
caagcagaagacggcatacgagat

SEQ ID NO: 9 - DNA, 16 bp, synthetic construct
  source 1..16: mol_type = other DNA
aaaccaggtggattag

SEQ ID NO: 10 - DNA, 16 bp, synthetic construct
  source 1..16: mol_type = other DNA
aaaccaggtgcaacgg

SEQ ID NO: 11 - DNA, 16 bp, synthetic construct
  source 1..16: mol_type = other DNA
aaaccaggtgagacta

SEQ ID NO: 12 - DNA, 16 bp, synthetic construct
  source 1..16: mol_type = other DNA
aaaccaggtcccggat

SEQ ID NO: 13 - DNA, 16 bp, synthetic construct
  source 1..16: mol_type = other DNA
aaaccaggtcacacca

SEQ ID NO: 14 - DNA, 16 bp, synthetic construct
  source 1..16: mol_type = other DNA
aaaccaggtcaattgc

SEQ ID NO: 15 - DNA, 16 bp, synthetic construct
  source 1..16: mol_type = other DNA
aaaccaggtaacgtga

SEQ ID NO: 16 - DNA, 16 bp, synthetic construct
  source 1..16: mol_type = other DNA
aaacaagcatagactg

SEQ ID NO: 17 - DNA, 16 bp, synthetic construct
  source 1..16: mol_type = other DNA
aaacaagcataatcgg

SEQ ID NO: 18 - DNA, 16 bp, synthetic construct
  source 1..16: mol_type = other DNA
aaacaagcaggcaact

SEQ ID NO: 19 - DNA, 16 bp, synthetic construct
  source 1..16: mol_type = other DNA
aaacaagcagaaaccc

SEQ ID NO: 20 - DNA, 16 bp, synthetic construct
  source 1..16: mol_type = other DNA
aaacaagcacttcaac

SEQ ID NO: 21 - DNA, 16 bp, synthetic construct
  source 1..16: mol_type = other DNA
aaacaagcactaatcc

SEQ ID NO: 22 - DNA, 16 bp, synthetic construct
  source 1..16: mol_type = other DNA
aaacaagcacgattct

SEQ ID NO: 23 - DNA, 16 bp, synthetic construct
  source 1..16: mol_type = other DNA
aaacaagcacctcgac

SEQ ID NO: 24 - DNA, 16 bp, synthetic construct
  source 1..16: mol_type = other DNA
aaacaagcacattgtc

SEQ ID NO: 25 - DNA, 16 bp, synthetic construct
  source 1..16: mol_type = other DNA
aaacaagcacagcaac

SEQ ID NO: 26 - DNA, 16 bp, synthetic construct
  source 1..16: mol_type = other DNA
aaacaagcaaggtcac

SEQ ID NO: 27 - DNA, 16 bp, synthetic construct
  source 1..16: mol_type = other DNA
aaacaagcaagggagc

SEQ ID NO: 28 - DNA, 16 bp, synthetic construct
  source 1..16: mol_type = other DNA
aaacaagcaaactaac